(19) United States
(12) Patent Application Publication
(10) Pub. No.: US 2024/0277834 A1
(43) Pub. Date: Aug. 22, 2024

(54) NUCLEIC ACID MOLECULES

(71) Applicant: ASTRAZENECA AB, Södertälje (SE)

(72) Inventors: Albert George THOM, Cambridge (GB); Joseph Richard FRANCICA, Gaithersburg, MD (US); Wade Stanton BLAIR, Gaithersburg, MD (US); Jason Paul LALIBERTE, Gaithersburg, MD (US); Julie Ann DOUTHWAITE, Cambridge (GB); Philip A. NEWTON, Cambridge (GB); Gareth John GRIFFIN, Cambridge (GB); Colin Harold HARDMAN, Cambridge (GB); Yueh-Ming LOO, Gaithersburg, MD (US)

(21) Appl. No.: 18/417,362

(22) Filed: Jan. 19, 2024

Related U.S. Application Data
(60) Provisional application No. 63/480,749, filed on Jan. 20, 2023.

(51) Int. Cl. A61K 39/215; A61P 31/14; A61P 37/04; C07K 14/47; C12N 9/10; C12N 9/12; C12N 9/42; A61K 39/00

(52) U.S. Cl. CPC A61K 39/215; A61P 31/14; A61P 37/04; C07K 14/47; C12N 9/1025; C12N 9/1096; C12N 9/12; C12N 9/2442; C12Y 203/03001; C12Y 206/01001; C12Y 207/11011; C12Y 302/01014; A61K 2039/6031; A61K 2039/6056; C07K 2319/30

(57) ABSTRACT

The present disclosure relates to a nucleic acid molecule comprising 5′-UTR and/or 3′-UTR sequences that yield high translation levels. Aspects of the disclosure further relate to nucleic acid molecules suitable for use as a vaccine in the treatment and prevention of infectious diseases, including those caused by a coronavirus, compositions comprising said nucleic acid molecules and methods of treating or preventing infectious diseases.

### CROSS-REFERENCE TO RELATED APPLICATIONS

This application claims benefit under 35 U.S.C. § 119(e) of U.S. Provisional Application No. 63/480,749, filed Jan. 20, 2023, which is incorporated by reference herein.

### REFERENCE TO SEQUENCE LISTING

This application incorporates by reference a Sequence Listing submitted electronically with the application as an XML file entitled “COFER-101-SequenceListing.xml” created on Jan. 18, 2024 and having a size of 91,906 bytes.

### FIELD

The present disclosure relates to a nucleic acid molecule comprising 5′-UTR and/or 3′-UTR sequences that yield high translation levels. Aspects of the disclosure further relate to nucleic acid molecules suitable for use as a vaccine in the treatment and prevention of infectious diseases, including those caused by a coronavirus.

### BACKGROUND

Nucleic acid vectors have multiple uses in both the treatment and study of human diseases. In molecular biology, nucleic acid vectors can be used to introduce disease-related genes or proteins of interest in cell or animal models as a means of assaying. Furthermore, there are now myriad therapeutic settings where expression of a nucleic acid sequences is desirable, including expression of a functional protein to treat a disease caused by a lack of said protein or as nucleic acid-based vaccines. Nucleic acid vectors generally comprise a simple structure, consisting of a coding sequence, encoding a peptide or protein of interest; 5′ and 3′-untranslated regions (UTRs), which aid in translation and stability of the vector; and a polyadenylation signal, which protects the vector from enzymatic degradation and plays a key role in translation. The constitutive parts of nucleic acid vectors can be modified to treat or model a specific disease.

Recent successes of nucleic acid vaccines during the COVID-19 pandemic has brought renewed interest to the field of nucleic acid-based therapies, particularly those for the expression of heterologous proteins of interest. For example, interest has now turned to pan-mRNA vaccines, where antigens from multiple disease-associated pathogens are administered simultaneously to provide pan-immunity against multiple seasonal pathogens (e.g., Clinical Trial NCT05596S734 evaluating mRNA vaccine candidates against COVID-19 and influenza).

Pan-vaccines would be greatly assisted by improvements to the nucleic acid vectors. For example, maximising or boosting translational efficiency from vectors might achieve high neutralisation responses against antigens at lower mRNA dose levels. This would enable multiple vectors encoding multiple antigens to be simultaneously administered without exceeding recommended mRNA dose amounts per vaccination. Moreover, boosting translational efficiency may lower costs associated with production of mRNA therapeutics by enabling efficacious responses at lower dose levels. Such an advantage would be applicable to any nucleic acid-based expressed biologic therapeutic approach (e.g., in vivo expressed antibodies, wild-type proteins, cancer neo-antigens, as well as traditional anti-pathogen vaccines).

### SUMMARY

The present disclosure relates to a nucleic acid molecule comprising at least one coding sequence flanked by and operably linked to 5′-and/or 3′-UTRs. The UTR(s) are engineered such that they increase translation of a protein of interest encoded for by the coding sequence. The coding sequence or sequences may encode for one or more peptides or fragments thereof of an infectious agent, such as the receptor binding domain or spike protein from one or more coronavirus variants. The molecule may also include the coding sequence of a multimerization unit, such that upon assembly a multimeric complex is formed.

Certain instances of the present disclosure are summarized below. This list is only exemplary and not exhaustive of all of the instances provided by this disclosure.
1. A nucleic acid molecule comprising a 5′ untranslated region (5′-UTR), a coding sequence and a 3′ untranslated region (3′-UTR), wherein the coding sequence is operably linked to the 5′-UTR and the 3′-UTR and wherein
   (i) the 5′-UTR comprises a sequence derived from a 5′-UTR of human chitinase-1 (CHIT1) and the 3′-UTR comprises a sequence derived from a 3′-UTR of human citrate synthase (CS);
   (ii) the 5′-UTR comprises a sequence derived from a 5′-UTR of human protein kinase cAMP-activated catalytic subunit beta (PRKACB) and the 3′-UTR comprises a sequence derived from a 3′-UTR of human chitinase-1 (CHIT1);
   (iii) the 5′-UTR comprises a sequence derived from a 5′-UTR of human protein kinase cAMP-activated catalytic subunit beta (PRKACB) and the 3′-UTR comprises a sequence derived from a 3′-UTR of human citrate synthase (CS);
   (iv) the 5′-UTR comprises a sequence derived from a 5′-UTR of human chitinase-1 (CHIT1) and the 3′-UTR comprises a sequence derived from a 3′-UTR of human chitinase-1 (CHIT1), wherein the coding sequence is not derived from human chitinase-1 (CHIT1);
   (v) the 5′-UTR comprises a sequence derived from a 5′-UTR of human glutamic-oxaloacetic transaminase 1 (GOT1) and the 3′-UTR comprises a sequence derived from a 3′-UTR of human citrate synthase (CS); or
   (vi) the 5′-UTR comprises a sequence derived from a 5′-UTR of human glutamic- oxaloacetic transaminase 1 (GOT1) and the 3′-UTR comprises a sequence derived from a 3′-UTR of human chitinase-1 (CHIT1).
2. The nucleic acid molecule of clause 1, wherein the nucleic acid molecule is a deoxyribonucleic (DNA) molecule and (i) the 5′-UTR comprises the sequence of SEQ ID NO: 1 or a sequence at least 80%, 85%, 90% or 95% identical thereto and the 3′-UTR comprises the sequence of SEQ ID NO: 9 or a sequence at least 80%, 85%, 90% or 95% identical thereto; (ii) the 5′-UTR comprises the sequence of SEQ ID NO: 3 or a sequence at least 80%, 85%, 90% or 95% identical thereto and the 3′-UTR comprises the sequence of SEQ ID NO: 11 or a sequence at least 80%, 85%, 90% or 95% identical thereto; (iii) the 5′-UTR comprises the sequence of SEQ ID NO: 3 or a sequence at least 80%, 85%, 90% or 95% identical thereto and the 3′-UTR comprises the sequence of SEQ ID NO: 9 or a sequence at least 80%, 85%, 90% or 95% identical thereto; (iv) the 5′-UTR comprises the sequence of SEQ ID NO: 1 or a sequence at least 80%, 85%, 90% or 95% identical thereto and the 3′-UTR comprises the sequence of SEQ ID NO: 11 or a sequence at least 80%, 85%, 90% or 95% identical thereto; (v) the 5′-UTR comprises the sequence of SEQ ID NO: 5 or a sequence at least 80%, 85%, 90% or 95% identical thereto and the 3′-UTR comprises the sequence of SEQ ID NO: 9 or a sequence at least 80%, 85%, 90% or 95% identical thereto; or (vi) the 5′-UTR comprises the sequence of SEQ ID NO: 5 or a sequence at least 80%, 85%, 90% or 95% identical thereto and the 3′-UTR comprises the sequence of SEQ ID NO: 11 or a sequence at least 80%, 85%, 90% or 95% identical thereto.
3. The nucleic acid molecule of clause 1, wherein the nucleic acid molecule is a ribonucleic acid molecule (RNA) and (i) the 5′-UTR comprises the sequence of SEQ ID NO: 2 or a sequence at least 80%, 85%, 90% or 95% identical thereto and the 3′-UTR comprises the sequence of SEQ ID NO: 10 or a sequence at least 80%, 85%, 90% or 95% identical thereto; (ii) the 5′-UTR comprises the sequence of SEQ ID NO: 4 or a sequence at least 80%, 85%, 90% or 95% identical thereto and the 3′-UTR comprises the sequence of SEQ ID NO: 12 or a sequence at least 80%, 85%, 90% or 95% identical thereto; (iii) the 5′-UTR comprises the sequence of SEQ ID NO: 4 or a sequence at least 80%, 85%, 90% or 95% identical thereto and the 3′-UTR comprises the sequence of SEQ ID NO: 10 or a sequence at least 80%, 85%, 90% or 95% identical thereto; (iv) the 5′-UTR comprises the sequence of SEQ ID NO: 2 or a sequence at least 80%, 85%, 90% or 95% identical thereto and the 3′-UTR comprises the sequence of SEQ ID NO: 12 or a sequence at least 80%, 85%, 90% or 95% identical thereto; (v) the 5′-UTR comprises the sequence of SEQ ID NO: 6 or a sequence at least 80%, 85%, 90% or 95% identical thereto and the 3′-UTR comprises the sequence of SEQ ID NO: 10 or a sequence at least 80%, 85%, 90% or 95% identical thereto; or (vi) the 5′-UTR comprises the sequence of SEQ ID NO: 6 or a sequence at least 80%, 85%, 90% or 95% identical thereto and the 3′-UTR comprises the sequence of SEQ ID NO: 12 or a sequence at least 80%, 85%, 90% or 95% identical thereto.
4. The nucleic acid molecule of clause 1 or clause 3, wherein the 5′-UTR comprises or consists of the sequence of SEQ ID NO: 2 and the 3′-UTR comprises or consists of the nucleic acid sequence of SEQ ID NO: 10.
5. The molecule of any preceding clause, wherein the coding sequence comprises a sequence encoding a therapeutic protein or peptide.
6. The molecule of clause 5, wherein the therapeutic protein or peptide is a wild-type sequence of a human protein.
7. The molecule of clause 5 or clause 6, wherein the therapeutic protein or peptide is an antibody, or antigen binding fragment thereof.
8. The molecule of clause 5, wherein the therapeutic protein or peptide comprises a disease-associated antigen (DAA).
9. The molecule of clause 8, wherein the disease-associated antigen is a tumour-associated antigen, a viral antigen or a bacterial antigen.
10. The molecule of clause 9, wherein the tumour-associated antigen is not expressed in normal tissue or is mutated in tumour cells.
11. The molecule of any one of clauses 1 and 3 to 10, wherein the nucleic acid molecule is mRNA.
12. The molecule according to any one of clauses 8 to 11, wherein the coding sequence further encodes a multimerization unit (MU).
13. The molecule of clause 12, wherein the multimerization unit (MU) is ferritin.
14. The molecule of clause 12 or clause 13, wherein the coding sequence further encodes a linker, optionally a glycine-serine linker.
15. The molecule of clause 14, wherein the linker is encoded between the DAA and the MU, thereby encoding a DAA-MU fusion protein.
16. The molecule according to any one of clauses 9 to 15, wherein the viral antigen is a coronavirus antigen.
17. The molecule according to clause 16, wherein the coronavirus is selected from SARS-CoV-1 and/or SARS-COV-2.
18. The molecule according to clause 16 or clause 17, wherein the coronavirus antigen is a coronavirus spike (S) protein, or antigenic fragment thereof.
19. The molecule according to clause 18, wherein the S protein is stabilised in a pre-fusion conformation.
20. The molecule according to clause 18 or clause 19, wherein the S protein comprises K986P and V987P mutations.
21. The molecule according to clause 18, wherein the antigenic fragment thereof is a receptor binding domain (RBD).
22. A composition comprising a first nucleic acid vector comprising the molecule of any one of clauses 11 to 21, wherein the disease-associated antigen is a Wuhan variant Spike (S) Protein or a Delta variant Spike (S) Protein.
23. The composition of clause 22, wherein the first nucleic acid vector comprises the antigen-linker-ferritin sequence set forth in SEQ ID NO: 23 or SEQ ID NO: 25.
24. The composition of any one of clauses 22 to 23, further comprising a second nucleic acid vector comprising a molecule of any one of clauses 111 to 21, wherein the disease-associated antigen is an Omicron variant Spike (S) Protein, optionally variant BA.2, BA.4/5 or XBB.1.5.
25. The composition of any one of clauses 22 to 24, wherein the first and/or second nucleic acid vector(s) comprises a polyadenylation sequence comprising between 60 and 100 adenine nucleotides.
26. The composition of any one of clauses 22 to 25, wherein the first and/or second nucleic acid vector(s) comprises N1-methylpseudouridine at 80% to 100% of uridine positions.
27. The composition of any one of clauses 22 to 26, wherein the first and/or nucleic acid vector(s) comprise a 5′-cap structure, optionally a cap 1 structure.
28. The composition of any one of clauses 22 to 27, wherein the first nucleic acid vector and/or the second nucleic acid vector comprises or consists of the 5′-UTR sequence set forth in SEQ ID NO: 19 and a 3′-UTR sequence set forth in SEQ ID NO: 21.
29. The composition of any one of clauses 24 to 28, wherein the second nucleic acid vector comprises the antigen-linker-ferritin sequence set forth in SEQ ID NO: 27.
30. The composition of any one of clauses 24 to 28, wherein the second nucleic acid vector comprises the antigen-linker-ferritin sequence set forth in SEQ ID NO: 42.
31. A composition comprising a nucleic acid vector comprising the molecule of any one of clauses 11 to 21, wherein the disease-associated antigen is an Omicron variant Spike (S) Protein, optionally variant BA.2, BA.4/5 or XBB.1.5.
32. The composition of clause 31, wherein the first nucleic acid vector comprises the antigen-linker-ferritin sequence set forth in SEQ ID NO: 27 or SEQ ID NO: 42.
33. The composition of any one of clauses 31 to 32, wherein the nucleic acid vector comprises a polyadenylation sequence comprising between 60 and 100 adenine nucleotides.
34. The composition of any one of clauses 31 to 33, wherein the nucleic acid vector comprises N1-methylpseudouridine at 80% to 100% of uridine positions.
35. The composition of any one of clauses 31 to 34, wherein the nucleic acid vector comprises a 5′-cap structure, optionally a cap 1 structure.
36. The composition of any one of clauses 31 to 35, wherein the nucleic acid vector and/or the second nucleic acid vector comprises or consists of the 5′-UTR sequence set forth in SEQ ID NO: 19 and a 3′-UTR sequence set forth in SEQ ID NO: 21.
37. The molecule or composition according to any one of clauses 11 to 36, wherein the nucleic acid vectors are formulated in lipid nanoparticles (LNPs).
38. The composition or nucleic acid molecule of any preceding clause for use in medicine.
39. A vaccine comprising the nucleic acid molecule or composition according to any preceding clause.
40. The composition or vaccine according to any one of clauses 22 to 39, for use in a method of preventing and/or treating an infectious disease, optionally a disease caused by a coronavirus.
41. A method of preventing and/or treating an infectious disease in a subject, the method comprising administering an effective amount of the composition or vaccine according to any one of clauses 22 to 39 to the subject optionally a disease caused by a coronavirus.
42. A method of vaccinating a subject against an infectious disease, optionally a disease caused by a coronavirus, comprising administering an effective amount of the composition or vaccine according to any one of clauses 22 to 39.
43. A nucleic acid vector comprising a 5′ cap 1 structure, a 5′-UTR sequence derived from a 5′-UTR of human chitinase-1 (CHIT1), a coding sequence encoding a Wuhan variant Spike (S) Protein fused with a ferritin sequence or a Delta variant Spike (S) Protein fused with a ferritin sequence and a 3′-UTR sequence derived from a 3′-UTR of human citrate synthase (CS) and a polyadenylation sequence comprising between 70 and 90 adenine nucleotides.
44. A nucleic acid vector comprising a 5′ cap 1 structure, a 5′-UTR sequence derived from a 5′-UTR of human chitinase-1 (CHIT1), a coding sequence encoding an Omicron variant BA.2, BA.4/5 or XBB.1.5 Spike (S) Protein fused with a ferritin sequence and a 3′-UTR sequence derived from a 3′-UTR of human citrate synthase (CS) and a polyadenylation sequence comprising between 70 and 90 adenine nucleotides.
45. A composition comprising the nucleic acid vector of clause 43 and the nucleic acid vector of clause 44.
46. A nucleic acid vector comprising the sequence set forth in SEQ ID NO: 36.
47. A nucleic acid vector comprising the sequence set forth in SEQ ID NO: 37.
48. A nucleic acid vector comprising the sequence set forth in SEQ ID NO: 38.
49 A nucleic acid vector comprising the sequence set forth in SEQ ID NO: 43.
50. A composition comprising the vector of clause 46 or clause 47 and the vector of clause 48 or clause 49.
51. A nucleic acid vector comprising a coding sequence that encodes for a polypeptide having the sequence set forth in SEQ ID NO: 39.
52. A nucleic acid vector comprising a coding sequence that encodes for a polypeptide having the sequence set forth in SEQ ID NO: 40.
53. A nucleic acid vector comprising a coding sequence that encodes for a polypeptide having the sequence set forth in SEQ ID NO: 41.
54. A nucleic acid vector comprising a coding sequence that encodes for a polypeptide having the sequence set forth in SEQ ID NO: 44.
55. The nucleic acid vector according to any of clauses 51 to 54, further comprising a 5′- UTR and a 3′-UTR as set out in any of clauses 1-4 or 28.
56. The nucleic acid vector according to any of clauses 51 to 55, which is an mRNA.
57. The nucleic acid vector of clause 56, wherein the mRNA comprises any of the features of clauses 26 to 28.
58. A composition comprising the vector of clause 51 and the vector of clause 52.
59. A composition comprising the vector of clause 51 or clause 52 and the vector of clause 53 or clause 54.

### DESCRIPTION OF THE FIGURES

Those of skill in the art will understand that the drawings, described below, are for illustrative purposes only. The drawings are not intended to limit the scope of the present teachings in any way.

FIG. 1 is a bar chart comparing the expression of eGFP encoding mRNA (in A549 cells) with a constant 3′-UTR region (albumin) and candidate 5′-UTRs (UTR-11,-37,-52,-53—identified in the examples). The black bar represents a vector with a control 5′-UTR region (HSD17B4). The selected 5′-UTRs increase the fluorescence intensity of eGFP in A549 cells. Candidate 5′-UTRs show the ability to increase eGFP expression in A549 cells, compared to a control 5′-UTR (HSD17B4). The data is representative of 3 independent experiments, with 3-4 technical replicates in each experiment.

FIG. 2 is a bar chart comparing the expression of eGFP encoding mRNA (in Hela cells) with a constant 3′-UTR region (albumin) and candidate 5′-UTRs (UTR-11,-37,-52,-53). The black bar represents a vector with a control 3′-UTR (albumin) and a control 5′-UTR (HSD17B4). Candidate 5′-UTRs show the ability to increase eGFP expression in Hela cells, compared to a control 5′-UTR (HSD17B4). The data is representative of 3 independent experiments, with 3-4 technical replicates in each experiment.

FIG. 3 is a bar chart comparing the expression of eGFP mRNA (in A549 cells) with candidate 3′-UTR regions (UTR-3,-4,-36—identified in the examples) and a control 5′-UTR region (HSD17B4). The black bar represents a control 3′-UTR (albumin) and a 5′-UTR (HSD17B4). Candidate 3′-UTRs can increase the expression of eGFP mRNA in A549 cells when compared to a control 3′-UTR. The data is representative of 2 independent experiments, with 3 technical replicates in each experiment.

FIG. 4 is a bar chart comparing the expression of eGFP mRNA (in Hela cells) with candidate 3′-UTRs (UTR-3,-4,-36) and a control 5′-UTR (HSD17B4). Candidate 3′-UTRs can increase the expression of eGFP mRNA in Hela cells, when compared to a control 3′-UTR. The black bar represents control 3′-UTR (albumin) and a control 5′-UTR (HSD17B4). The data is representative of 2 independent experiments, with 3 technical replicates in each experiment.

FIG. 5 is a bar chart comparing the expression of eGFP mRNA (in A549 cells) with combinations of candidate 3′-UTRs (CHIT-1, CS) and 5′-UTRs (GOT1, PRKACB, CHIT1) to a control consisting of a control 3′-UTR (albumin) and a control 5′-UTR (HSD17B4). The black bar represents eGFP mRNA expression in the control. Combining a candidate 3′-UTR with a candidate 5′-UTR can increase eGFP mRNA expression in A549 cells. The data is representative of 3 independent experiments, with 3 technical replicates in each experiment.

FIG. 6 is a bar chart demonstrating the expression of eGFP encoding modified mRNA (in A549 cells) with a combination of candidate 5′ (GOT1, PRKACB, CHIT) and 3′-UTRs (CHIT, CS). The black bar represents the expression of eGFP encoding modified mRNA with control 5′ (HSD17B4) and 3′-UTR (albumin) regions. The combinations of candidate 5′-UTRs and candidate 3′-UTRs increase the expression of eGFP encoding modified mRNA, when compared to expression with control 5′ and 3-UTRs. The data is representative of 3 independent experiments, with 3 technical replicates in each experiment.

FIG. 7 shows the antibody expression in A549 cells with scFv-Fc encoding mRNA with a combination of candidate or control 5′ and 3′-UTRs. The black bar represents the expression of scFv-Fc encoding mRNA with a control 5′-UTR (HSD17B4) and a control 3′-UTR (Albumin). A combination of PRKACB(5′-UTR)/CHIT(3′-UTR) or CHIT(5′-UTR)/CS(3′-UTR) yields higher expression of scFv-Fc encoding mRNA than expression with control 5′ and 3′-UTR regions. The data is representative of 3 independent experiments, with 3 technical replicates in each experiment.

FIG. 8 shows the antibody expression in A549 cells with scFv-Fc encoding modified mRNA (in A549 cells) with a combination of candidate or control 5′ and 3′-UTRs. Modified mRNA contains modified uridine (5-methoxyuridine). The black bar represents the expression of scFv-Fc encoding mRNA with a control 5′-UTR (HSD17B4) and a control 3′-UTR (Albumin). Combinations of PRKACB(5′-UTR)/CHIT(3′-UTR), PRKACB(5′-UTR)/CS(3′-UTR), CHIT(5′-UTR)/CHIT(3′-UTR) and CHIT(5′-UTR)/CS(3′-UTR) yield higher expression of scFv-Fc encoding mRNA than expression with control 5′ and 3′-UTR regions. The data is representative of 3 independent experiments, with 3 technical replicates in each experiment.

FIG. 9A-B shows that mRNAs employing the 5′-UTR CHIT1 and 3′-UTR CS UTR generated the highest levels of EGFP expression among both BHK-21 (FIG. 9A) and HEK293 (FIG. 9B) cells compared to two competitor mRNA molecules. mRNA_AZ and mRNA comp A and mRNA comp B all comprise 100% pseudo U. An mRNA_AZ with 0% pseudo was included for comparative purposes.

FIG. 10 shows improved pan-variant immunogenicity in mice of an mRNA molecule according to the disclosure, encoding Delta antigen fused to ferritin via a linker compared to encoding a native spike.

FIG. 11 shows improved pan-variant immunogenicity in non-human primates of an mRNA molecule according to the disclosure, encoding Delta antigen fused to ferritin via a linker compared to encoding a native spike.

### DETAILED DESCRIPTION

All references referred to are incorporated herein by reference in their entireties.

Many modifications and other instances of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific instances disclosed and that modifications and other instances are intended to be included within the scope of the appended claims. Although specific terms are employed herein, they are used in a generic and descriptive sense only and not for purposes of limitation.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

### Definitions

The term “nucleic acid sequence” is intended to encompass a polymer of DNA or RNA, i.e., a polynucleotide, which can be single-stranded or double-stranded and which can contain non-natural or altered nucleotides e.g. modified uridine. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double-and single-stranded DNA, and double- and single-stranded RNA. The terms include, as equivalents, analogs of either RNA or DNA made from nucleotide analogs and modified polynucleotides such as, though not limited to, methylated and/or capped polynucleotides. Nucleic acids are typically linked via phosphate bonds to form nucleic acid sequences or polynucleotides, though many other linkages are known in the art (e.g. phosphorothioates, boranophosphates, and the like).

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulphide bond formation, glycosylation, lipidation, acetylation, phosphorylation, or any other manipulation or modification, such as conjugation with a labelling component. Also included within the definition are, for example, polypeptides containing one or more analogs of an amino acid (including, for example, unnatural amino acids, etc.), as well as other modifications known in the art. It is understood that, because the polypeptides of this disclosure are based upon antibodies, in some aspects, the polypeptides can occur as single chains or associated chains.

“Percent identity” refers to the extent of identity between two sequences (e.g. nucleic acid sequences). Percent identity can be determined by aligning two sequences, introducing gaps to maximize identity between the sequences. Percent identity should generally be calculated between the same types of nucleic acids, i.e. for DNA sequences or RNA sequences. Thus, it is understood, if a DNA sequence “corresponds to” an RNA sequence or if an RNA sequence “corresponds to” a DNA sequence, in a first step the RNA sequence is converted into the corresponding DNA sequence (in particular by replacing the uracils (U) by thymidines (T) throughout the sequence) or, vice versa, the DNA sequence is converted into the corresponding RNA sequence (in particular by replacing the T by U throughout the sequence). Alignments can be generated using programs known in the art. For purposes herein, alignment of nucleotide sequences can be performed with the blastn program set at default parameters (see National Center for Biotechnology Information (NCBI): ncbi.nlm.nih.gov). Identity can be calculated between two sequences can be calculated by multiplying the number of matches in the pair by 100 and dividing by the length of the aligned region, including gaps. Identity scoring only counts perfect matches. Gaps at the end of sequences are not included, and internal gaps are included in the length.

“5′-untranslated region (5′-UTR)” has the usual meaning recognised by a skilled person. It is the region of a nucleic acid molecule located 5′ of a coding sequence and which is not translated into protein. A 5′-UTR usually starts with the transcriptional start site and end before the start codon of the coding sequence.

“3′-untranslated region (3′-UTR)” has the usual meaning recognised by a skilled person. It is the region of a nucleic acid molecule located 3′ of a coding sequence and which is not translated into protein. A 3′-UTR is usually 3′ of a coding sequence. If the molecule comprises a polyadenylation signal, the 3′-UTR is usually between the coding sequence and the polyadenylation signal.

“Coding sequence” is a continuous stretch of DNA or RNA beginning with a start codon (e.g., methionine (ATG or AUG)) and ending with a stop codon (e.g., TAA, TAG or TGA, or UAA, UAG or UGA). A coding sequence typically encodes a polypeptide. The coding sequences disclosed herein are operably linked to the5′ and 3′ UTRs described herein.

“Messenger RNA (mRNA)” is any RNA that encodes a (at least one) protein (a naturally-occurring, non-naturally-occurring, or modified polymer of amino acids) and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents RNA (e.g., mRNA), the “T”s would be substituted for “U”s. Thus, any of the DNAs disclosed and identified by a particular sequence identification number herein also disclose the corresponding RNA (e.g., mRNA) sequence complementary to the DNA, where each “T” of the DNA sequence is substituted with “U.”

A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). Nucleic acids can comprise a region or regions of linked nucleosides. Such regions 5 may have variable backbone linkages. The linkages can be standard phosphodiester linkages, in which case the nucleic acids would comprise regions of nucleotides (a “nucleotide” refers to a nucleoside, including a phosphate group).

“Expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing) and (3) translation of an RNA into a polypeptide or protein.

The term “pharmaceutical composition” refers to a preparation which is in such form as to permit the biological activity of the active ingredient to be effective, and which contains no additional components which are unacceptably toxic to a subject to which the composition would be administered. The composition can be sterile.

As used herein, the terms “subject” and “patient” are used interchangeably. The subject can be an animal. In some aspects, the subject is a mammal such as a non-human animal (e.g. cow, pig, horse, cat, dog, rat, mouse, monkey or other primate, etc.). In some aspects, the subject is a human.

As used in the present disclosure and claims, the singular forms “a”, “an”, and “the” include plural forms unless the context clearly dictates otherwise.

It is understood that wherever aspects are described herein with the language “comprising”, otherwise analogous aspects described in terms of “consisting of’ and/or “consisting essentially of” are also provided. In this disclosure, “comprises”, “comprising”, “containing” and “having” and the like can mean “includes”, “including”, and the like; “consisting essentially of” or “consists essentially” are open-ended, allowing for the presence of more than that which is recited so long as basic or novel characteristics of that which is recited is not changed by the presence of more than that which is recited, but excludes prior art aspects.

Unless specifically stated or obvious from context, as used herein, the term “or” is understood to be inclusive. The term “and/or” as used in a phrase such as “A and/or B” herein is intended to include both “A and B,” “A or B”, “A”, and “B”. Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Any molecules, vectors, compositions, uses or methods provided herein can be combined with one or more of any of the other molecules, vectors, compositions, uses or methods provided herein.

### Untranslated Regions (UTRs)

The present disclosure provides a nucleic acid molecule comprising a 5′ untranslated region (5′-UTR), a coding sequence and a 3′ untranslated region (3′-UTR), wherein the coding sequence is operably linked to the 5′-UTR and the 3′-UTR and wherein
(i) the 5′-UTR comprises, or consists of, a sequence derived from a 5′-UTR of human chitinase-1 (CHIT1) and the 3′-UTR comprises, or consists of, a sequence derived from a 3′- UTR of human citrate synthase (CS);
(ii) the 5′-UTR comprises, or consists of, a sequence derived from a 5′-UTR of human protein kinase cAMP-activated catalytic subunit beta (PRKACB) and the 3′-UTR comprises, or consists of, a sequence derived from a 3′-UTR of human chitinase-1 (CHIT1);
(iii) the 5′-UTR comprises, or consists of, a sequence derived from a 5′-UTR of human protein kinase cAMP-activated catalytic subunit beta (PRKACB) and the 3′-UTR, or consists of, a sequence derived from a 3′-UTR of human citrate synthase (CS);
(iv) the 5′-UTR comprises, or consists of, a sequence derived from a 5′-UTR of human chitinase-1 (CHIT1) and the 3′-UTR comprises, or consists of, a sequence derived from a 3′-UTR of human chitinase-1 (CHIT1), wherein the coding sequence is not derived from human chitinase-1 (CHIT1);
(v) the 5′-UTR comprises, or consists of, a sequence derived from a 5′-UTR of human glutamic-oxaloacetic transaminase 1 (GOT1) and the 3′-UTR comprises, or consists of, a sequence derived from a 3′-UTR of human citrate synthase (CS); or
(vi) the 5′-UTR comprises, or consists of, a sequence derived from a 5′-UTR of human glutamic-oxaloacetic transaminase 1 (GOT1) and the 3′-UTR comprises, or consists of, a sequence derived from a 3′-UTR of human chitinase-1 (CHIT1).

The nucleic acid molecule of the disclosure is a nucleic acid vector.

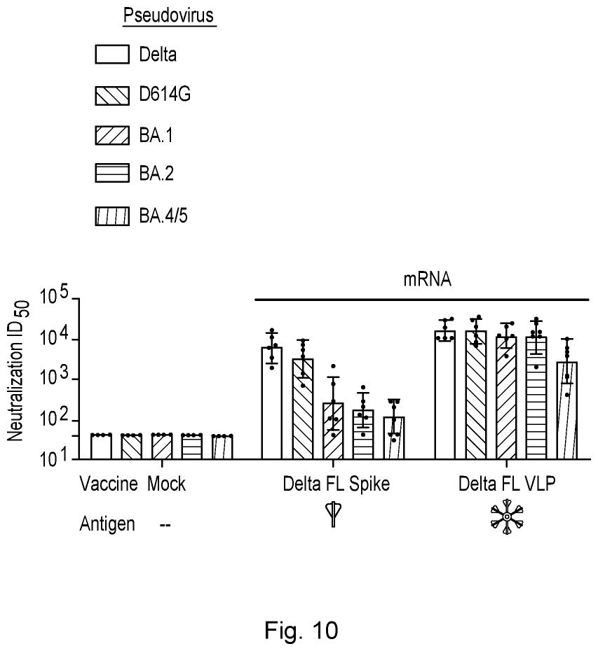

The 5′-UTR and 3′-UTR sequences of the disclosure are particularly useful for increasing translation levels of the coding sequence . . . . The examples demonstrate that the combination of 5′-and 3′-UTRs disclosed herein enhance translation levels of multiple proteins of interest, including GFP and an scFv-Fc (e.g., a therapeutic protein). The translation levels were greater than achieved compared to clinically validated UTR combinations derived from regulatory approved mRNA vaccine products (see FIG. 10, for example). Therefore, the disclosure provides a nucleic acid molecule capable of enhancing translation of a protein of interest, potentially paving the way for providing lower dosing regimens for mRNA vaccines as can be achieved in the art at present. This may enable development of combination mRNA vaccines against multiple pathogens administrable as a single dose. Furthermore, the UTR combinations described herein may be preferential for use in conjunction with therapeutic proteins to be expressed in vivo, such as antibodies (or variants thereof) targeting intracellular targets. The UTRs disclosed herein may enhance the dynamic range of in vivo expression yields, thus enabling exploration of a greater dose range to identify the most efficacious dose.

Levels of translation obtained with the UTRs of the present disclosure can be assessed by any appropriate assay available to a skilled person, for example, by measuring the concentration of a protein of interest encoded by the coding sequence, for example by detecting a marker protein, such as GFP, or a therapeutic protein such as an scFv-Fc or a vaccine antigen, such as a SARS-COV-2 spike protein.

The 5′-UTRs and 3′-UTRs of the disclosure are capable of increasing translation of a coding sequence, optionally in HeLa or A549 cells, compared to a 5′-UTR sequence derived from albumin, optionally comprising or consisting of the sequence set forth in SEQ ID NO:16 or a corresponding RNA sequence, and a 3′-UTR sequence derived from HSD17B4, optionally comprising or consisting of the sequence set forth in SEQ ID NO:17, or a corresponding RNA sequence.

In one instance, the 5′-UTR derived from a 5′-UTR of CHIT1 does not comprise a sequence comprising ATG, optionally the 5′-UTR derived from a 5′-UTR of CHIT1 does not comprise a sequence consisting of ATGGGCTGCAGCCTGCCGCTGA (SEQ ID NO: 35), or the corresponding RNA sequence.

The present disclosure further provides a deoxyribonucleic (DNA) molecule comprising a 5′-UTR, a coding sequence and a 3′-UTR, wherein the coding sequence is operably linked to the 5′-UTR and the 3′-UTR and wherein
(i) the 5′-UTR comprises, or consists of, the sequence of SEQ ID NO: 1 or a sequence at least 80%, 85%, 90% or 95% identical thereto and the 3′-UTR comprises, or consists of, the sequence of SEQ ID NO: 9 or a sequence at least 80%, 85%, 90% or 95% identical thereto;
(ii) the 5′-UTR comprises, or consists of, the sequence of SEQ ID NO: 3 or a sequence at least 80%, 85%, 90% or 95% identical thereto and the 3′-UTR comprises, or consists of, the sequence of SEQ ID NO: 11 or a sequence at least 80%, 85%, 90% or 95% identical thereto;
(iii) the 5′-UTR comprises, or consists of, the sequence of SEQ ID NO: 3 or a sequence at least 80%, 85%, 90% or 95% identical thereto and the 3′-UTR comprises, or consists of, the sequence of SEQ ID NO: 9 or a sequence at least 80%, 85%, 90% or 95% identical thereto;
(iv) the 5′-UTR comprises, or consists of, the sequence of SEQ ID NO: 1 or a sequence at least 80%, 85%, 90% or 95% identical thereto and the 3′-UTR comprises, or consists of, the sequence of SEQ ID NO: 11 or a sequence at least 80%, 85%, 90% or 95% identical thereto;
(v) the 5′-UTR comprises, or consists of, the sequence of SEQ ID NO: 5 or a sequence at least 80%, 85%, 90% or 95% identical thereto and the 3′-UTR comprises, or consists of, the sequence of SEQ ID NO: 9 or a sequence at least 80%, 85%, 90% or 95% identical thereto; or
(vi) the 5′-UTR comprises, or consists of, the sequence of SEQ ID NO: 5 or a sequence at least 80%, 85%, 90% or 95% identical thereto and the 3′-UTR, or consists of, the sequence of SEQ ID NO: 11 or a sequence at least 80%, 85%, 90% or 95% identical thereto.

The present disclosure further provides a ribonucleic (RNA) molecule comprising a 5′-UTR, a coding sequence and a 3′-UTR, wherein the coding sequence is operably linked to the 5′-UTR and the 3′-UTR and wherein
(i) the 5′-UTR comprises, or consists of, the sequence of SEQ ID NO: 2 or a sequence at least 80%, 85%, 90% or 95% identical thereto and the 3′-UTR comprises, or consists of, the sequence of SEQ ID NO: 10 or a sequence at least 80%, 85%, 90% or 95% identical thereto;
(ii) the 5′-UTR comprises, or consists of, the sequence of SEQ ID NO: 4 or a sequence at least 80%, 85%, 90% or 95% identical thereto and the 3′-UTR comprises, or consists of, the sequence of SEQ ID NO: 12 or a sequence at least 80%, 85%, 90% or 95% identical thereto;
(iii) the 5′-UTR comprises, or consists of, the sequence of SEQ ID NO: 4 or a sequence at least 80%, 85%, 90% or 95% identical thereto and the 3′-UTR comprises, or consists of, the sequence of SEQ ID NO: 10 or a sequence at least 80%, 85%, 90% or 95% identical thereto;
(iv) the 5′-UTR comprises, or consists of, the sequence of SEQ ID NO: 2 or a sequence at least 80%, 85%, 90% or 95% identical thereto and the 3′-UTR comprises, or consists of, the sequence of SEQ ID NO: 12 or a sequence at least 80%, 85%, 90% or 95% identical thereto;
(v) the 5′-UTR comprises, or consists of, the sequence of SEQ ID NO: 6 or a sequence at least 80%, 85%, 90% or 95% identical thereto and the 3′-UTR comprises, or consists of, the sequence of SEQ ID NO: 10 or a sequence at least 80%, 85%, 90% or 95% identical thereto; or
(vi) the 5′-UTR comprises, or consists of, the sequence of SEQ ID NO: 6 or a sequence at least 80%, 85%, 90% or 95% identical thereto and the 3′-UTR comprises, or consists of, the sequence of SEQ ID NO: 12 or a sequence at least 80%, 85%, 90% or 95% identical thereto.

In one instance, the nucleic acid molecule of the present disclosure comprises a 5′-UTR comprising, or consisting of, the sequence of SEQ ID NO:19 or a sequence at least 80%, 85%, 90% or 95% identical thereto and a 3′-UTR comprising, or consisting of, the sequence of SEQ ID NO: 21 or a sequence at least 80%, 85%, 90% or 95% identical thereto. The present disclosure further provides a nucleic acid molecule comprising a 5′-UTR, a coding sequence and a 3′-UTR,
wherein the 5′-UTR comprises a sequence derived from a 5′-UTR of any one of human chitinase-1 (CHIT1), glucuronidase beta (GUSB1), human protein kinase cAMP-activated catalytic subunit beta (PRKACB), glutamic-oxaloacetic transaminase 1 (GOT1) and/or the 3′-UTR comprises a sequence derived from a 3′-UTR of any one of human chitinase-1 (CHIT1), pyruvate kinase L/R (PKLR) or human citrate synthase (CS),
wherein the coding sequence is operably linked to the 5′-UTR and the 3′-UTR and wherein the coding sequence is not CHIT1, GUSB1, PRKACB, GOT1, PKLR or CS.

The examples show that these 5′-UTRs and/or these 3′UTRs are capable of increasing translation of the coding sequence as assessed in accordance with this disclosure.

The present disclosure further provides a deoxyribonucleic acid molecule comprising a 5′-UTR, a coding sequence and 3′-UTR, wherein the 5′-UTR comprises a sequence selected from SEQ ID NO: 1, SEQ ID NO: 3, SEQ ID NO: 5 or SEQ ID NO: 7 or a sequence at least 80%, 85%, 90% or 95% identical thereto and/or the 3′-UTR comprises a sequence selected from SEQ ID NO: 9, SEQ ID NO: 11 or SEQ ID NO: 12 or a sequence at least 80%, 85%, 90% or 95% identical thereto.

The present disclosure further provides a ribonucleic acid molecule comprising a 5′-UTR, a coding sequence and 3′-UTR, wherein the 5′-UTR comprises a sequence selected from SEQ ID NO: 2, SEQ ID NO: 4, SEQ ID NO: 6 or SEQ ID NO: 8 or a sequence at least 80%, 85%, 90% or 95% identical thereto and/or the 3′-UTR comprises a sequence selected from SEQ ID NO: 10, SEQ ID NO: 12 or SEQ ID NO: 14 or a sequence at least 80%, 85%, 90% or 95% identical thereto.

In one instance, the 5′-UTR of the present disclosure comprises a sequence at least 80%, 85%, 90% or 95% identical to a sequence selected from any one of SEQ ID NOS: 1 to 8, wherein the sequence is capable of increasing translation of the coding sequence, optionally in HeLa cells or A549 cells, compared to a reference 5′-UTR, optionally the HSD17B4 5′-UTR (SEQ ID NO: 17), when constant 3′-UTR sequences are included in both molecules. Constant 3′-UTR sequences mean the same 3′-UTR sequence is paired with the 5′-UTR of the present disclosure and the reference 5′-UTR.

In one instance, the 3′-UTR of the present disclosure comprises a sequence at least 80%, 85%, 90% or 95% identical to a sequence selected from any one of SEQ ID NOS: 9 to 14, wherein the sequence is capable of increasing translation of the coding sequence, optionally in Hela cells or A549 cells, compared to a reference 3′-UTR, optionally the albumin 3′-UTR (SEQ ID NO: 16), when constant 5′-UTR sequences are included in both molecules. Constant 5′-UTR sequences mean the same 5′-UTR sequence is paired with the 3′-UTR of the present disclosure and the reference 3′-UTR.

In one instance, the nucleic acid molecule of the disclosure comprises a 5′-UTR comprising a sequence at least 80%, 85%, 90% or 95% identical to a sequence selected from SEQ ID NO: 1, or the corresponding RNA sequence, and a 3′-UTR comprising a sequence at least 80%, 85%, 90% or 95% identical to a sequence selected from SEQ ID NO: 9, or the corresponding RNA sequence, wherein the 5′-UTR sequence and the 3′-UTR sequence are capable of increasing translation of the coding sequence, optionally in Hela cells or A549 cells, compared to translation by a nucleic acid molecule comprising a reference 5′-UTR, optionally the HSD17B4 5′-UTR (SEQ ID NO: 17, or the corresponding RNA sequence), and a reference 3′-UTR, optionally the albumin 3′-UTR (SEQ ID NO: 16, or the corresponding RNA sequence), operably linked to the coding sequence.

In one instance, the 5′-UTR consists of a sequence selected from any one of SEQ ID NOS: 1 to 8 or a sequence at least 80%, 85%, 90% or 95% identical thereto, where the sequence is capable of increasing translation of the coding sequence as assessed in accordance with this disclosure, and/or the 3′-UTR consists of a sequence selected from any one of SEQ ID NOS: 9 to 14 or a sequence at least 80%, 85%, 90%, 95% or 98% identical thereto, where the sequence is capable of increasing translation of the coding sequence as assessed in accordance with this disclosure.

In one instance, the nucleic acid molecule comprises a 5′-UTR, a coding sequence and a 3′-UTR, wherein the coding sequence is operably linked to the 5′-UTR and 3′-UTR and wherein the 5′-UTR comprises a sequence derived from the human chitinase-1 (CHIT1) 5′-UTR, optionally a sequence according to SEQ ID NO: 1 or SEQ ID NO: 2, or a sequence at least 80%, 85%, 90% or 95% identical thereto. The examples show that this 5′-UTR is capable of increasing translation of the coding sequence as assessed in accordance with this disclosure.

In one instance, the nucleic acid molecule comprises a 5′-UTR, a coding sequence and a 3′-UTR, wherein the coding sequence is operably linked to the 5′-UTR and 3′-UTR and wherein the 3′-UTR comprises a sequence derived from the human citrate synthase (CS) 3′ UTR, optionally a sequence according to SEQ ID NO: 9 or SEQ ID NO: 10, or a sequence at least 80%, 85%, 90% or 95% identical thereto. The examples show that this 3′-UTR is capable of increasing translation of the coding sequence as assessed in accordance with this disclosure.

In one instance, the nucleic acid molecule comprises a 5′-UTR, a coding sequence and a 3′-UTR, wherein the coding sequence is operably linked to the 5′-UTR and 3′-UTR and wherein the 5′-UTR comprises a sequence derived from the human chitinase 1(CHIT1) 5′ UTR and wherein the 3′-UTR comprises a sequence derived from the human citrate synthase (CS) 3′-UTR. The examples show that a nucleic acid molecule comprising these 5′ and 3′-UTRs yield the highest translation levels of multiple proteins of interest in multiple cell lines compared to clinically validated 5′ and 3′-UTR combinations.

In one instance, the nucleic acid molecule comprises a 5′-UTR, a coding sequence and a 3′-UTR, wherein the coding sequence is operably linked to the 5′-UTR and 3′-UTR and wherein the 5′-UTR comprises a sequence derived from the human protein kinase CAMP-activated catalytic subunit beta (PRKACB) 5′ UTR, optionally a sequence according to SEQ ID NO: 3 or SEQ ID NO: 4, or a sequence at least 80%, 85%, 90% or 95% identical thereto, that is capable of increasing expression of the coding sequence as assessed in accordance with this disclosure.

In one instance, the nucleic acid molecule comprises a 5′-UTR, a coding sequence and a 3′-UTR, wherein the coding sequence is operably linked to the 5′-UTR and 3′-UTR and wherein the 3′-UTR comprises a sequence derived from the human chitinase-1 (CHIT1) 3′-UTR, optionally a sequence according to SEQ ID NO: 11 or SEQ ID NO: 12, or a sequence at least 80%, 85%, 90% or 95% identical thereto, that is capable of increasing expression of the coding sequence as assessed in accordance with this disclosure.

In one instance, the nucleic acid molecule comprises a 5′-UTR, a coding sequence and a 3′-UTR, wherein the coding sequence is operably linked to the 5′-UTR and 3′-UTR and wherein the 5′-UTR comprises a sequence derived from the human protein kinase cAMP-activated catalytic subunit beta (PRKACB) 5′-UTR, that is capable of increasing expression of the coding sequence as assessed in accordance with this disclosure and wherein the 3′-UTR comprises a sequence derived from the chitinase-1 (CHIT1) 3′-UTR, optionally a sequence according to SEQ ID NO: 5, that is capable of increasing expression of the coding sequence as assessed in accordance with this disclosure.

In one instance, the nucleic acid molecule comprises a 5′-UTR, a coding sequence and a 3′-UTR, wherein the coding sequence is operably linked to the 5′-UTR and 3′-UTR and wherein the 5′-UTR comprises a sequence derived from the glutamic-oxaloacetic transaminase 1 (GOT1) 5′ UTR, optionally a sequence according to SEQ ID NO: 5 or SEQ ID NO: 6, or a sequence at least 80%, 85%, 90% or 95% identical thereto, that is capable of increasing expression of the coding sequence as assessed in accordance with this disclosure.

In one instance, the nucleic acid molecule comprises a 5′-UTR, a coding sequence and a 3′-UTR, wherein the coding sequence is operably linked to the 5′-UTR and 3′-UTR and wherein the 5′-UTR comprises a sequence derived from the glucuronidase beta (GUSB1) 5′ UTR, optionally a sequence according to SEQ ID NO: 7 or SEQ ID NO: 8, or a sequence at least 80%, 85%, 90% or 95% identical thereto, that is capable of increasing expression of the coding sequence as assessed in accordance with this disclosure.

In one instance, the nucleic acid molecule comprises a 5′-UTR, a coding sequence and a 3′-UTR, wherein the coding sequence is operably linked to the 5′-UTR and 3′-UTR and wherein the 3′-UTR comprises a sequence derived from the pyruvate kinase L/R (PKLR) sequence, optionally a sequence according to SEQ ID NO: 13 or SEQ ID NO: 14, or a sequence at least 80%, 85%, 90% or 95% identical thereto, that is capable of increasing expression of the coding sequence as assessed in accordance with this disclosure.

Table 1 sets out the UniProt (Release 2022_04) codes for the genes referred to in accordance with the disclosure:

TABLE 1

| Gene Name | UniProt code | Organism | Full Protein Name |
|---|---|---|---|
| CHIT1 | Q13231 • | Human | Chitinase 1/ |
| | CHIT1_HUMAN | | Chitotriosidase-1 |
| CS | O75390 • | Human | Citrate synthase |
| | CISY_HUMAN | | |
| GUSB | P08236 • | Human | Glucuronidase beta |
| | BGLR_HUMAN | | |
| PKLR | P30613 • | Human | pyruvate kinase L/R |
| | KPYR_HUMAN | | |
| PRKACB | P22694 • | Human | Protein Kinase CAMP- |
| | KAPCB_HUMAN | | activated catalytic |
| | | | subunit beta |
| GOT1 | P17174 • | Human | glutamic-oxaloacetic |
| | AATC_HUMAN | | transaminase 1 |

In an instance, the 5′-UTR and/or 3′-UTR sequences of the disclosure include sequences 85%, 86%, 87%, 88%, 89%, 90%, 91%, 91%, 92%, 93%, 94%, 95%, 96%, 97%, 98% or 99% identical to the sequences disclosed herein, where the sequences are capable of increasing expression of the coding sequence as assessed in accordance with this disclosure.

The nucleic acid molecule of the disclosure can be, for example, a plasmid, an episome, a cosmid or a phage. Suitable vectors and methods of vector preparation are well known in the art (see, e.g., Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994)).

In one instance, the nucleic acid molecule of the present disclosure is a closed circular molecule or a linear molecule.

In various instances, the nucleic acid molecule according to the disclosure comprises, in a 5′ to 3′ direction of transcription, a promoter, a 5′-UTR and a 3′-UTR flanking a coding sequence and a polyadenylation signal.

In one instance, the nucleic acid molecule of the disclosure further comprises a 5′-cap structure, optionally a cap1 structure. Further suitable cap structures and approaches for generating suitable cap structures are disclosed in WO2017/053297 and Tusup et al., Design of in vitro Transcribed mRNA Vectors for Research and Therapy, Chim Int J Chem. 2019; 73(5):391-394, both of which are hereby incorporated by reference. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; 35 G(5′)ppp(5′)G; m7G(5′)ppp(5′)A; m7G(5′)ppp(5′)G (New England BioLabs, Ipswich, MA). 5′-capping of modified RNA may be completed post-transcriptionally using a Vaccinia Virus Capping Enzyme to generate the “Cap 0” structure: m7G(5′)ppp(5′)G (New England BioLabs, Ipswich, MA). Cap 1 structure may be generated using both Vaccinia Virus Capping Enzyme and a 2′-0 methyl-transferase to generate: m7G(5′)ppp(5′)G-2′-O-methyl. Cap 2 structure may be generated from the Cap 1 structure followed by the 2′-O-methylation of the 5′antepenultimate nucleotide using a 2′-0 methyl-transferase. Cap 3 structure may be generated from the Cap 2 structure followed by the 2′-O-methylation of the 5′-preantepenultimate nucleotide using a 2′-0 methyl-transferase. Enzymes may be derived from a recombinant source. Further suitable means for generating suitable cap structures are disclosed in WO2016/193226, which is hereby incorporated by reference.

In one instance, the nucleic acid molecule of the disclosure comprises a promoter that is any promoter for a DNA-dependent RNA polymerase. For example, T7 (optionally comprising or consisting of the sequence TAATACGACTCACTATAAGG (SEQ ID NO: 15), T3, SP6 or Syn5 RNA polymerases.

In some instances, the nucleic acid molecule disclosed herein comprises a polyadenylation signal (Poly A tail). The Poly A tail is a long sequence of adenine residues which lies at the 3′ end of the molecule. The role of the Poly A tail is two-fold. The Poly A tail is essential for translation, with Poly(A) binding proteins (PABP) recruiting translation factors to enhance translation levels. Furthermore, the Poly A tail increases the stability of a nucleic acid molecule by PABP binding poly(A) in mRNA and protecting it against exonuclease digestion. In mRNA, the poly A tail is also known to play a key role in the transport of mRNA from the nucleus to the ribosomes (Shlake, T., et al., RNA Biol., (2012), 9(11), 1319-1330). In one instance, the nucleic acid molecule of the disclosure comprises a Poly A tail of about 50 to about 500 adenosine nucleotides. For example, the poly A tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosines. In some instances, the poly A tail contains 50 to 250 adenosines. In some instances, the poly A tail contains 60 to 100 adenosines. In some instances, the poly A tail contains 70, 71, 72, 73, 74, 75, 76, 77, 78, 79 or 80 adenosines. In some instances, the poly A tail contains 77 adenosines.

In one instance of the disclosure, the nucleic acid molecule comprises a split Poly(A) tail. A split Poly(A) tail can comprise at least two adenosine containing elements, optionally of between 30 and 60 adenosines each, separated by a spacer optionally of between 1 and 25 nucleotides.

In one instance, the nucleic acid molecule of the disclosure is a ribonucleic acid (RNA) molecule.

In one instance, the ribonucleic acid (RNA) molecule is mRNA.

### Leader Sequences

In some instances, the coding sequence disclosed herein comprises a leader sequence. A leader sequence may encode a signal peptide. In some instances, the signal peptide is fused to the expressed therapeutic protein. In such instances, the leader sequence and the gene of interest are within the same open reading frame (ORF).

Signal peptides, comprising the N-terminal 15-60 amino acids of proteins, are typically needed for the translocation across the membrane on the secretory pathway and control entry of most proteins to the secretory pathway. In eukaryotes, the signal peptide of a nascent precursor protein (pre-protein) directs the ribosome to the rough endoplasmic reticulum (ER) and initiates the transport of the growing peptide chain across it for processing. ER processing produces mature proteins, in which the signal peptide is typically cleaved by resident signal peptidases, at least for secreted proteins.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some instance, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids. In some instances, the signal peptide has the following sequence: MPLLLLLPLLWAGALA (SEQ ID NO: 34).

### Nucleosides and Nucleotides

In some instances, the nucleic acid molecule provided herein is not chemically modified and comprises the standard nucleotides adenine (A), thymine (T) or uracil (U), where the nucleic acid molecule is an RNA, guanine (G) or cytosine (C).

In some instances, the nucleic acid molecule comprises modified nucleotides. Many modified nucleotides are known in the art, such as disclosed in WO2007/024708, which is hereby incorporated by reference. Modifications can include either naturally occurring modifications or non-naturally occurring modifications. Modifications can include those at the sugar, backbone or nucleobase protein of the nucleotide and/or nucleoside as well known in the art.

In some instances, the nucleic acid molecules herein may include natural (i.e., standard) nucleotide or nucleoside, non-naturally or naturally occurring modified nucleotides or nucleosides, or any combination thereof.

In one instance, wherein the nucleic acid molecule is an RNA, the RNA may comprise standard A, G and C nucleotides and modified U nucleotides.

In some instances, the nucleic acid molecule comprising the modified nucleoside or nucleotide (e.g., a “modified RNA nucleic acid molecule”) exhibits reduced immunogenicity in a cell or organism relative to an unmodified RNA nucleic molecule comprising the same sequence.

In some instances, modified nucleosides in nucleic acid molecules provided herein (e.g., RNA nucleic acid molecules such as mRNA) comprise N1-methyl-pseudouridine (m1Ψ), 1-ethyl-pseudouridine (e1Ψ), 5-methoxy-uridine (mo5U), 5-methyl-cytidine (m5C), and/or pseudouridine (Ψ). In some instances, modified nucleotides in nucleic acid molecules (e.g., RNA nucleic acid molecules, such as mRNA) comprise 5-methoxymethyl uridine, 5-methylthio uridine, 1-methoxymethyl pseudouridine, 5-methyl cytidine, and/or 5-methoxy cytidine. In some instances, the RNA nucleic acid molecule includes a combination of at least two (e.g., 2, 3, 4 or more) of any of the aforementioned modified nucleobases.

In some instances, the nucleic acid molecule provided herein comprises N1-methyl-pseudouridine (m1Ψ) at one or more or all uridine positions of the nucleic acid molecule.

In some instances, the nucleic acid molecule comprises 5-methoxy-uridine (mo5U) at one or more or all uridine positions of the nucleic acid molecule.

In some instances, the nucleic acid molecule comprises from about 1% to about 100% modified nucleotides (either in relation to overall nucleotide content or in relation to one or more types of nucleotide (i.e., any one or more of A, G, U, T or C). In some instances, the nucleic acid molecule comprises any intervening percent of modified nucleotide content. For example, from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%. The remaining percentage is accounted for by unmodified A, G, U, T or C.

The nucleic acid molecules may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some instances, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some instances, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

In some instances, the nucleic acid molecules is an mRNA in which the uridine is replaced by a compound having a single unique structure. In some instances, the single unique structure is N1-methyl-pseudouridine. In some instances, the nucleic acid molecule comprises at least 5%, at least 10%, at least 25%, at least 50%, at least 80%, at least 90% or 100% N1-methyl-pseudouridine.

In one instance the mRNA comprises a modified nucleobase. In some instances, the modified nucleobase is modified adenine (A), cytosine (C), uracil (U) and guanine (G).

In one instance, the modified nucleobase is modified U. In some instances, the modified U is 1-methylpseudouridine (m1Ψ) and pseudouridine (Ψ), such as is disclosed in WO2007/024708, which is hereby incorporated by reference.

In one instance, the nucleic acid molecules of the present disclosure comprise UTR sequences comprising 5-methoxy-uridine (mo5U) at one or more or all uridine positions of the nucleic acid molecule. The molecule can comprise at least 25% ratio of modified uridine to unmodified uridine, including 25% to 50%, or at least 50%.

The examples show that modifying uridine in a 5′-UTR sequence derived from the human CHIT1 5′-UTR and in a 3′-UTR sequence derived from the human citrate synthase (CS) 3′-UTR results in a particularly substantial increase in translation.

In one instance, the nucleic acid molecules of the present disclosure comprise sequences comprising N1-methyl-pseudouridine (m1Ψ) at one or more or all uridine positions of the nucleic acid molecule. The molecule can comprise at least 75% ratio of modified uridine to unmodified uridine, including 100%.

### Coding Sequence

In one instance, the nucleic acid molecule of the disclosure comprises a coding sequence encoding a therapeutic protein or peptide, optionally a wild-type sequence of a human protein or an antibody, or antigen binding fragment thereof.

Thus, the nucleic acid molecule of the disclosure can be useful in gene therapies.

In some instances, the coding sequence is not CHIT1, GUSB1, PRKACB, GOT1, PKLR or CS.

In one instance, the nucleic acid molecule of the disclosure comprises a coding sequence encoding a disease-associated antigen (DAA). This molecule of the disclosure is a vaccine vector.

### Vaccine Vector

Nucleic acid vaccine vectors pose substantial advantages over conventional vaccination approaches. In terms of safety, RNA based vaccines are non-infectious, unlike live or live attenuated vaccination approaches. Furthermore, RNA vaccines do not integrate into the genome and therefore are not a risk for mutagenesis. Moreover, both DNA and RNA-based vaccines have proved highly efficacious against many infectious agents, including Zika, Influenza, Rabies and SARS-COV-2. Nucleic acid-based vaccines offer a cheap, fast and easily-scalable alternative to conventional vaccination approaches (Pardi, N., et al., Nature Reviews, (2018), 17, 261-279).

In one instance of the disclosure, the disease-associated antigen can be a viral antigen, a bacterial antigen or a tumour-associated antigen.

After entering a cell, the coding sequence of a DNA molecule can undergo transcription and translation, or the coding sequence of an RNA molecule can undergo translation to produce the antigenic protein or fragments thereof of an antigen.

Upon production of the antigen, the exposure of the host immune system to the protein or protein fragment can stimulate an immune response. This immune response may comprise the stimulation of antibody production by B cells and the production of memory B cells, capable of producing antibodies against the antigen or fragment thereof of a specified infectious agent. Upon infection with the same infectious agent, the host immune system is primed against this antigen or antigenic protein fragment, reducing the time-span of the immune response against the infectious agent. Therefore, reducing or preventing the onset of symptoms in response to infection.

In one instance of the disclosure, the nucleic acid molecule comprises a sequence encoding a nano antigen particle. The antigen may be an antigen as described anywhere herein.

In one instance, the nucleic acid molecule comprises a coding sequence that encodes a multimerization unit. In one instance, the multimerization unit is a ferritin protein. The multimerization unit may be the scaffold for the nano antigen particle. In some instances, the ferritin is a Helicobacter pylori ferritin. In some instances, the nucleic acid molecule comprises a sequence that encodes an antigenic protein and a ferritin protein, wherein the antigenic protein and ferritin assemble to form a nano antigen particle.

In one instance of the disclosure, the coding sequence further encodes a linker. The linker can be encoded between the DAA and the multimerization unit, optionally a ferritin protein, such that the DAA is fused to the multimerization unit in the encoded molecule.

In one instance, the nucleic acid molecule comprises RNA 5′- and 3′-UTR sequences and RNA coding sequence, optionally mRNA sequence.

### Coronavirus Vaccine

The recent Covid-19 pandemic has led to an urgent need for improved vaccines that target coronaviruses of concern. To date several variants of SARS-COV-2 have been identified with some of the most infectious among these being the Delta and Omicron variants. Vaccines currently approved for the treatment of SARS-COV-2 all encompass stimulating immunity against the SARS-COV-2 spike protein. Although, a number of mutations in the receptor binding domain of the spike protein have been identified in new variants of SARS-COV-2, which is thought to have led to increased vaccine resistance in newly emerging variants (Zhao, J. et al., Environmental research, (2022), 206(112240)). Research estimates that current vaccines are around three to five fold less potent against the Delta variant than the Alpha variant of SARS-COV-2 (Planas, D. et al., Nature, (2021), 596, 276-280).

Accordingly, there is an ongoing need for improved vaccines in general, including those useful in the prevention and treatment of coronaviruses.

Accordingly, one instance of the disclosure provides a nucleic acid molecule as described herein encoding a coronavirus (CoV) antigen. In one instance, the nucleic acid molecule comprises RNA 5′-and 3′-UTR sequences and RNA coding sequence. In one instance, the coronavirus antigen can be selected from SARS-COV-1 and/or SARS-COV-2. In one instance, the nucleic acid molecule encodes a SARS-COV-2 antigen selected from one or more of the following variants: Wuhan, Alpha, Beta, Delta and Omicron, optionally BA.1, BA.2, BA.2.86, BA.3, BA.4/5, BQ.1, BQ.1.1, JN.1, XBB.1 and XBB.1.5.

The coronavirus virion comprises a large number of glycosylated spike (S) proteins projecting from the surface of the virion. These S proteins form trimer structures, and mediate virus entry into host cells, making it a primary target for vaccine design.

The coronavirus spike protein is 1273 amino acids in length and comprises a signal peptide, and S1 and S2 subunits. The S1 subunit contains a receptor-binding domain (RBD) that recognizes and binds to a specific host cell receptor, angiotensin-converting enzyme 2 (ACE2). The S2 subunit mediates viral cell membrane fusion.

Thus, in one instance, the nucleic acid molecule comprises a sequence encoding an S protein or antigenic fragment thereof. Upon delivery into a host cell, the S protein is translated and processed in the host cell, resulting in the presentation of a trimerized S protein on a host cell surface.

In some instances, the nucleic acid molecule comprises a sequence that encodes a CoV S protein and a ferritin protein, wherein the CoV S protein and ferritin assemble to form a nano antigen particle.

The S protein can be stabilised in a pre-fusion conformation. Further, the S protein can comprise K986P and/or V987P mutations.

In one instance, the nucleic acid molecule of the present disclosure encodes an antigenic fragment thereof that is a receptor binding domain (RBD). In one instance, the antigenic fragment is a RBD of a SARS-COV-2 S protein. In one instance, the antigenic fragment is a RBD of a SARS-COV-1 S protein.

In some instances, it may be advantageous to fuse a fragment of an antigenic protein to the ferritin in the nano antigen particle. In one instance, the fragment of the antigenic protein may be the RBD. The RBDs in the nano antigen particle may be derived from antigens of the same infectious agent i.e. be monovalent, or the RBDs of the nano antigen particle may be derived from antigens of more than one infectious agent i.e. be multivalent.

### Multimerization Units

The nucleic acid molecules as provided herein, in some instances, encode fusion proteins that comprise vaccine antigens linked to multimerization units. In some instances, such multimerization units impart desired properties to an antigen encoded by the nucleic acid molecule. For example, the examples show that multimerization units improve the immunogenicity of an antigen (e.g., the COVID spike protein), as compared to the immunogenicity of the same antigen expressed without the multimerization units. Furthermore, the multimerization units provided herein improve the pan-variant response against an antigen. For example, a nucleic acid molecule provided herein comprising a coding sequence encoding a COVID spike protein-multimerization unit fusion protein elicits a broader immune response against SARs-COV-2 variants compared to spike proteins alone.

In some instances, the multimerization unit is a protein that can self-assemble into protein nanoparticles that are highly symmetric, stable, and structurally organized, with diameters of 10-150 nm, a highly suitable size range for optimal interactions with various cells of the immune system. In some instances, viral proteins or virus-like particles can be used to form stable nanoparticle structures. Examples of such viral proteins are known in the art. For example, in some instances, the multimerization units is a hepatitis B surface antigen (HBsAg). HBsAg forms spherical particles with an average diameter of ˜22 nm and which lacked nucleic acid and hence are non-infectious (Lopez-Sagaseta, J. et al. Computational and Structural Biotechnology Journal 14 (2016) 58-68). In some instances, the multimerization unit is a hepatitis B core antigen (HBcAg) self-assembles into particles of 24-31 nm diameter, which resembled the viral cores obtained from HEY-infected human liver. HBcAg produced in self-assembles into two classes of differently sized nanoparticles of 300 A and 360 A diameter, corresponding to 180 or 240 protomers. In some instances, the antigen is fused to HBSAG or HBcAG to facilitate self-assembly of nanoparticles displaying the antigen.

In some instances, the multimerization unit is selected from the following self-assembling proteins: ferritin, lumazine synthase and encapsuling.

Ferritin is a protein whose main function is intracellular iron storage. Ferritin is made of 24 subunits, each composed of a four-alpha-helix bundle, that self-assemble in a quaternary structure with octahedral symmetry (Cho K. J. et al. J Mol Biol. 2009; 390:83-98). Several high resolution structures of ferritin have been determined, confirming that Helicobacter pyloriferritin is made of 24 identical protomers, whereas in animals, there are ferritin light and heavy chains that can assemble alone or combine with different ratios into particles of 24 subunits (Granier T. et al. J Biol Inorg Chem. 2003; 8:105-111; Lawson D. M. et al. Nature. 1991; 349:541-544). Ferritin self-assembles into nanoparticles with robust thermal and chemical stability. Thus, the ferritin nanoparticle is well-suited to carry and expose antigens.

Lumazine synthase (LS) is also well-suited as a nanoparticle platform for antigen display. LS, which is responsible for the penultimate catalytic step in the biosynthesis of riboflavin, is an enzyme present in a broad variety of organisms, including archaea, bacteria, fungi, plants, and eubacteria (Weber S. E. Flavins and Flavoproteins. Methods and Protocols, Series: Methods in Molecular Biology. 2014). The LS monomer is 150 amino acids long, and consists of beta-sheets along with tandem alpha-helices flanking its sides. A number of different quaternary structures have been reported for LS, illustrating its morphological versatility: from homopentamers up to symmetrical assemblies of 12 pentamers forming capsids of 150 A diameter. Even LS cages of more than 100 subunits have been described (Zhang X. et al. J Mol Biol. 2006; 362:753-770).

Encapsulin, a novel protein cage nanoparticle isolated from thermophile Thermotoga maritima, may also be used as a platform to present antigens on the surface of self-assembling nanoparticles. Encapsulin is assembled from 60 copies of identical 31 kDa monomers having a thin and icosahedral T=1 symmetric cage structure with interior and exterior diameters of 20 and 24 nm, respectively (Sutter M. et al. Nat Struct Mol Biol. 2008, 15: 939-947). Although the exact function of encapsulin in T. maritima is not clearly understood yet, its crystal structure has been recently solved and its function was postulated as a cellular compartment that encapsulates proteins such as DyP (Dye decolorizing peroxidase) and Flp (Ferritin like protein), which are involved in oxidative stress responses (Rahmanpour R. et al. FEES J. 2013, 280: 2097-2104).

In some instances, the nucleic acid molecule provided herein comprises a coding sequence encoding a coronavirus antigen (e.g., a SARS-COV-2 spike (S) protein) fused to a ferritin sub-unit.

### Linkers

In some instances, the nucleic acid molecules disclosed herein encode fusion proteins. In such instances, the each of the domains of the fusion protein (e.g., the antigen and the multimerization unit), may be separated by a coding sequence encoding a linker sequences. The linker sequence may be self-cleaving. In other words, the linker may be a self-cleavable linker. In other instances, the linker may be a protease-sensitive linker. In some instances, the linker may be a glycine-serine linker.

In some instances, the self-cleaving linker is selected from an F2A linker, P2A linker, T2A linker, E2A linker, and combinations thereof. This family of self-cleaving peptide linkers, referred to as 2A peptides, has been described in the art (see for example, Kim, J. H. et al. (2011) PLOS ONE 6:e18556).

In some instances, the glycine-serine linker has the following amino acid sequence: GSGGSG (SEQ ID NO: 28). In some instances, the glycine-serine linker is encoded by SEQ ID NO: 29 or SEQ ID NO: 30.

The skilled person will appreciate that other art-recognized linkers may be suitable for use in the constructs of the disclosure (e.g., encoded by the nucleic acid molecules provided herein). The skilled person will likewise appreciate that other polycistronic constructs (nucleic acid molecules encoding more than one antigen/polypeptide separately within the same molecule) may be suitable for use as provided herein.

### Manufacturing

The nucleic acid vaccine vectors of the present disclosure can be manufactured according to in vitro transcription. In vitro transcription of RNA is known in the art and is described in International Publication WO2014/152027, which is incorporated by reference herein in its entirety. In some instances, the RNA of the present disclosure is prepared in accordance with any one or more of the methods described in WO2018/053209 and WO2019/036682, each of which is incorporated by reference herein. In overview, a DNA template is generated, typically as a linearized plasmid, followed by in vitro transcription to synthesize the RNA, alongside or followed by capping.

The 5′ cap can be added by a multi-step enzymatic reaction or via co-transcription. In co-transcriptional capping, a cap analog, such as CleanCap® AG, is added directly to the in vitro transcription mixture. Alternatively, enzymatic capping using vaccinia virus capping enzyme is performed separately to the in vitro transcription.

Following purification, the mRNA product can be encapsulated in a lipid nanoparticle (LNP).

### Compositions

The present disclosure also provides a pharmaceutical composition comprising a nucleic acid molecule or LNP as defined anywhere herein and a pharmaceutical carrier.

In one instance, the pharmaceutical composition is a monovalent composition comprising a nucleic acid molecule according to the disclosure encoding a first antigen, or an immunogenic fragment or immunogenic variant thereof.

In one instance, the pharmaceutical composition is a bivalent composition comprising a further nucleic acid molecule according to the disclosure encoding a second antigen, or an immunogenic fragment or immunogenic variant thereof, wherein the second antigen is different to the first antigen.

The present disclosure further provides a composition comprising a first nucleic acid molecule according to the disclosure, wherein the disease-associated antigen is a Delta variant S protein. In one instance, the disease-associated antigen of a first nucleic acid molecule is a Wuhan variant S protein.

In one instance, the composition according to the disclosure can further include a second nucleic acid molecule encoding an Omicron variant S Protein, optionally variant BA.1, BA.2, BA.2.86, BA.3, BA.4/5, BQ.1, BQ.1.1, JN.1, XBB.1 or XBB.1.5.

In one instance, the second nucleic acid molecule encodes Omicron variant S Protein BA.4/5.

In one instance, the second nucleic acid molecule encodes Omicron variant S Protein XBB.1.5.

In one instance of the disclosure, the composition comprises:
1) a first nucleic acid molecule comprising a 5′-UTR comprising a sequence at least 95% identical to the sequence of SEQ ID NO: 19 and a 3′-UTR comprising a sequence at least 95% identical to the sequence of SEQ ID NO: 21, operably linked to a coding sequence, wherein the coding sequence comprises a sequence encoding a Delta variant S protein, a linker and a ferritin protein, wherein the encoded protein is a Delta variant S protein—ferritin fusion protein, and
2) a second nucleic acid molecule comprising a 5′-UTR comprising a sequence at least 95% identical to the sequence of SEQ ID NO: 19 and a 3′-UTR comprising a sequence at least 95% identical to the sequence of SEQ ID NO: 21, operably linked to a coding sequence, wherein the coding sequence comprises a sequence encoding an Omicron variant S protein, a linker and a ferritin protein, wherein the encoded protein is an Omicron variant S protein—ferritin fusion protein.

In one instance of the disclosure, the composition comprises:
1) a first nucleic acid molecule comprising a 5′-UTR comprising or consisting of the sequence of SEQ ID NO: 19 and a 3′-UTR comprising or consisting of the sequence of SEQ ID NO: 21, operably linked to a coding sequence, wherein the coding sequence comprises a sequence encoding a Delta variant S protein, a linker and a ferritin protein, wherein the encoded protein is a Delta variant S protein—ferritin fusion protein, and
2) a second nucleic acid molecule comprising a 5′-UTR comprising or consisting of the sequence of SEQ ID NO: 19 and a 3′-UTR comprising or consisting of the sequence of SEQ ID NO: 21, operably linked to a coding sequence, wherein the coding sequence comprises a sequence encoding an Omicron variant S protein, a linker and a ferritin protein, wherein the encoded protein is an Omicron variant S protein—ferritin fusion protein.

In one instance of the disclosure, the first nucleic acid molecule comprises a coding sequence as set forth in SEQ ID NO: 25 and/or the second nucleic acid molecule comprises a coding sequence as set forth in SEQ ID NO:27.

In one instance of the disclosure, the first nucleic acid molecule comprises a coding sequence as set forth in SEQ ID NO: 25 and/or the second nucleic acid molecule comprises a coding sequence as set forth in SEQ ID NO:42.

In one instance of the disclosure, the first nucleic acid molecule comprises a sequence as set forth in SEQ ID NO:37 and the second nucleic acid molecule comprises a sequence as set forth in SEQ ID NO:38. In one instance, the first nucleic acid molecule consists of a sequence as set forth in SEQ ID NO:37 and the second nucleic acid molecule consists of a sequence as set forth in SEQ ID NO:38.

In one instance of the disclosure, the first nucleic acid molecule comprises a sequence as set forth in SEQ ID NO:37 and the second nucleic acid molecule comprises a sequence as set forth in SEQ ID NO:43. In one instance, the first nucleic acid molecule consists of a sequence as set forth in SEQ ID NO:37 and the second nucleic acid molecule consists of a sequence as set forth in SEQ ID NO:43.

In one instance, the first nucleic acid molecule comprises a coding sequence that encodes for the polypeptide sequence set forth in SEQ ID NO: 40 and the second nucleic acid molecule comprises a coding sequence that encodes for the polypeptide sequence set forth in SEQ ID NO: 41.

In one instance, the first nucleic acid molecule contains a coding sequence that encodes for the polypeptide sequence set forth in SEQ ID NO: 40 and the second nucleic acid molecule contains a coding sequence that encodes for the polypeptide sequence set forth in SEQ ID NO: 41.

In one instance, the first nucleic acid molecule comprises a coding sequence that encodes for the polypeptide sequence set forth in SEQ ID NO: 40 and the second nucleic acid molecule comprises a coding sequence that encodes for the polypeptide sequence set forth in SEQ ID NO: 44.

In one instance, the first nucleic acid molecule contains a coding sequence that encodes for the polypeptide sequence set forth in SEQ ID NO: 40 and the second nucleic acid molecule contains a coding sequence that encodes for the polypeptide sequence set forth in SEQ ID NO: 44.

In one instance of the disclosure, the composition comprises:
1) a first nucleic acid molecule comprising a 5′-UTR comprising a sequence at least 95% identical to the sequence of SEQ ID NO: 19 and a 3′-UTR comprising a sequence at least 95% identical to the sequence of SEQ ID NO: 21, operably linked to a coding sequence, wherein the coding sequence comprises a sequence encoding a Wuhan variant S protein, a linker and a ferritin protein, wherein the encoded protein is a Wuhan variant S protein—ferritin fusion protein, and
2) a second nucleic acid molecule comprising a 5′-UTR comprising a sequence at least 95% identical to the sequence of SEQ ID NO: 19 and a 3′-UTR comprising a sequence at least 95% identical to the sequence of SEQ ID NO: 21, operably linked to a coding sequence, wherein the coding sequence comprises a sequence encoding an Omicron variant S protein, a linker and a ferritin protein, wherein the encoded protein is an Omicron variant S protein—ferritin fusion protein.

In one instance of the disclosure, the composition comprises:
1) a first nucleic acid molecule comprising a 5′-UTR comprising or consisting of the sequence of SEQ ID NO: 19 and a 3′-UTR comprising or consisting of the sequence of SEQ ID NO: 21, operably linked to a coding sequence, wherein the coding sequence comprises a sequence encoding a Wuhan variant S protein, a linker and a ferritin protein, wherein the encoded protein is a Wuhan variant S protein—ferritin fusion protein, and
2) a second nucleic acid molecule comprising a 5′-UTR comprising or consisting of the sequence of SEQ ID NO: 19 and a 3′-UTR comprising or consisting of the sequence of SEQ ID NO: 21, operably linked to a coding sequence, wherein the coding sequence comprises a sequence encoding an Omicron variant S protein, a linker and a ferritin protein, wherein the encoded protein is an Omicron variant S protein—ferritin fusion protein.

In one instance of the disclosure, the first nucleic acid molecule comprises a coding sequence as set forth in SEQ ID NO:23 and the second nucleic acid molecule comprises a coding sequence as set forth in SEQ ID NO:27.

In one instance of the disclosure, the first nucleic acid molecule comprises a coding sequence as set forth in SEQ ID NO:23 and the second nucleic acid molecule comprises a coding sequence as set forth in SEQ ID NO:42.

In one instance of the disclosure, the first nucleic acid molecule comprises a sequence as set forth in SEQ ID NO:36 and the second nucleic acid molecule comprises a sequence as set forth in SEQ ID NO:38. In one instance, the first nucleic acid molecule consists of a sequence as set forth in SEQ ID NO:36 and the second nucleic acid molecule consists of a sequence as set forth in SEQ ID NO:38.

In one instance of the disclosure, the first nucleic acid molecule comprises a sequence as set forth in SEQ ID NO:36 and the second nucleic acid molecule comprises a sequence as set forth in SEQ ID NO:43. In one instance, the first nucleic acid molecule consists of a sequence as set forth in SEQ ID NO:36 and the second nucleic acid molecule consists of a sequence as set forth in SEQ ID NO:43.

In one instance, the first nucleic acid molecule comprises a coding sequence that encodes for the polypeptide sequence set forth in SEQ ID NO: 39 and the second nucleic acid molecule comprises a coding sequence that encodes for the polypeptide sequence set forth in SEQ ID NO: 41.

In one instance, the first nucleic acid molecule contains a coding sequence that encodes for the polypeptide sequence set forth in SEQ ID NO: 39 and the second nucleic acid molecule contains a coding sequence that encodes for the polypeptide sequence set forth in SEQ ID NO: 41.

In one instance, the first nucleic acid molecule comprises a coding sequence that encodes for the polypeptide sequence set forth in SEQ ID NO: 39 and the second nucleic acid molecule comprises a coding sequence that encodes for the polypeptide sequence set forth in SEQ ID NO: 44.

In one instance, the first nucleic acid molecule contains a coding sequence that encodes for the polypeptide sequence set forth in SEQ ID NO: 39 and the second nucleic acid molecule contains a coding sequence that encodes for the polypeptide sequence set forth in SEQ ID NO: 44.

In one instance, the first and/or second nucleic acid molecule of any composition of the disclosure comprises 80% to 100% N1-methyl-pseudouridine (m1Ψ) at uridine positions of the nucleic acid molecule.

In one instance, the first and/or nucleic acid vector(s) comprise a 5′-cap structure, optionally a cap 1 structure.

In one instance, the first and/or second nucleic acid molecule of the composition comprises a T7 promoter sequence, optionally the RNA sequence corresponding to the sequence set forth in SEQ ID NO: 15.

In one instance, the first and/or second nucleic acid molecule of the composition encodes a glycine-serine linker, optionally with the sequence of SEQ ID NO: 28.

In one instance, the first and/or second nucleic acid molecule of the composition comprises a Poly A tail of between 70 and 90 adenosine nucleotides.

In one instance, the first and/or second nucleic acid molecule of the composition comprise a leader sequence. The leader sequence will be cleaved from the mature, expressed antigen. In one instance, the leader sequence encodes the following amino acid sequence: MPLLLLLPLLWAGALA (SEQ ID NO: 34).

The present disclosure further provides a monovalent composition comprising a nucleic acid molecule according to the disclosure, wherein the disease-associated antigen is an Omicron variant S Protein, optionally variant BA.1, BA.2, BA.2.86, BA.3, BA.4/5, BQ.1, BQ.1.1, JN.1, XBB.1 or XBB.1.5.

In one instance, the nucleic acid molecule encodes Omicron variant S Protein BA.4/5.

In one instance, the nucleic acid molecule encodes Omicron variant S Protein XBB.1.5.

In one instance of the disclosure, the composition comprises a nucleic acid molecule comprising a 5′-UTR comprising a sequence at least 95% identical to the sequence of SEQ ID NO: 19 and a 3′-UTR comprising a sequence at least 95% identical to the sequence of SEQ ID NO: 21, operably linked to a coding sequence, wherein the coding sequence comprises a sequence encoding an Omicron variant S protein, a linker and a ferritin protein, wherein the encoded protein is a Delta variant S protein—ferritin fusion protein.

In one instance of the disclosure, the composition comprises a nucleic acid molecule comprising a 5′-UTR comprising or consisting of the sequence of SEQ ID NO: 19 and a 3′-UTR comprising or consisting of the sequence of SEQ ID NO: 21, operably linked to a coding sequence, wherein the coding sequence comprises a sequence encoding an Omicron S protein, a linker and a ferritin protein, wherein the encoded protein is a Delta variant S protein—ferritin fusion protein.

In one instance of the disclosure, the nucleic acid molecule comprises a coding sequence as set forth in SEQ ID NO:27.

In one instance of the disclosure, the nucleic acid molecule comprises a coding sequence as set forth in SEQ ID NO:42.

In one instance of the disclosure, the nucleic acid molecule comprises a sequence as set forth in SEQ ID NO:38. In one instance, the nucleic acid molecule consists of a sequence as set forth in SEQ ID NO:38.

In one instance of the disclosure, the nucleic acid molecule comprises a sequence as set forth in SEQ ID NO:43. In one instance, the nucleic acid molecule consists of a sequence as set forth in SEQ ID NO:43.

In one instance, the nucleic acid molecule comprises a coding sequence that encodes for the polypeptide sequence set forth in SEQ ID NO: 41.

In one instance, the nucleic acid molecule contains a coding sequence that encodes for the polypeptide sequence set forth in SEQ ID NO: 41.

In one instance, the nucleic acid molecule comprises a coding sequence that encodes for the polypeptide sequence set forth in SEQ ID NO: 44.

In one instance, the nucleic acid molecule contains a coding sequence that encodes for the polypeptide sequence set forth in SEQ ID NO: 44.

In one instance, the nucleic acid molecule of the composition comprises 80% to 100% N1-methyl-pseudouridine (m1Ψ) at uridine positions of the nucleic acid molecule.

In one instance, nucleic acid vector comprises a 5′-cap structure, optionally a cap 1 structure.

In one instance, the nucleic acid molecule of the composition comprises a T7 promoter sequence, optionally the RNA sequence corresponding to the sequence set forth in SEQ ID NO: 15.

In one instance, the nucleic acid molecule of the composition encodes a glycine-serine linker, optionally with the sequence of SEQ ID NO: 28.

In one instance, the nucleic acid molecule of the composition comprises a Poly A tail of between 70 and 90 adenosine nucleotides.

In one instance, the nucleic acid molecule of the composition comprises a leader sequence. The leader sequence will be cleaved from the mature, expressed antigen. In one instance, the leader sequence encodes the following amino acid sequence: MPLLLLLPLLWAGALA (SEQ ID NO: 34).

In all instances of the disclosure, the sequence encoding the antigen may be further optimized via mutation to increase protein stability (such as the structure of the CoV spike protein or the RBD), maximise protein translation and reduce unwanted side effects.

Said compositions may comprise an effective amount of the nucleic acid molecule as defined herein. An effective amount of the nucleic acid molecule to be employed therapeutically will depend, for example, upon the therapeutic objectives, the route of administration, and the condition of the patient. In one instance, the effective amount of the nucleic acid molecule as defined anywhere herein within the pharmaceutical composition is effective to treat or prevent a disease associated with coronavirus infection.

The composition is a pharmaceutically acceptable (e.g., physiologically acceptable) composition, which comprises a carrier, preferably a pharmaceutically acceptable (e.g., physiologically acceptable) carrier. The pharmaceutically acceptable carrier may include one or more excipients. Pharmaceutically acceptable excipients are known and include carriers, excipients, or stabilizers that are nontoxic to the cell or mammal being exposed thereto at the dosages and concentrations employed. Any suitable carrier can be used within the context of the disclosure, and such carriers are well known in the art. The choice of carrier will be determined, in part, by the particular site to which the composition may be administered and the particular method used to administer the composition. The physiologically acceptable excipient may be an aqueous pH buffered solution. Examples of physiologically acceptable excipients include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as Ethylenediaminetetraacetic acid (EDTA); sugar alcohols such as mannitol or sorbitol; salt-forming counterions such as sodium; and/or nonionic surfactants such as TWEEN™, polyethylene glycol (PEG), and PLURONICS™.

The composition optionally can be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

The compositions can be administered intravenously. The composition can also be administered parenterally or subcutaneously.

Methods of administering a pharmaceutical composition as defined herein include, but are not limited to, parenteral administration (e.g., intradermal, intramuscular, intraperitoneal, intravenous and subcutaneous), epidural, and mucosal (e.g., intranasal and oral routes). In a specific example, a pharmaceutical composition is administered intranasally, intramuscularly, intravenously, or subcutaneously. The compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. Each dose may or may not be administered by an identical route of administration.

### Delivery System

Various delivery systems are known and can be used to administer a prophylactic or therapeutic agent (e.g., a nucleic acid molecule as disclosed herein), including, but not limited to, encapsulation in liposomes, microparticles, microcapsules, construction of a nucleic acid as part of a retroviral or other vector, etc. In addition, pulmonary administration can also be employed, e.g., by use of an inhaler or nebulizer, and formulation with an aerosolizing agent.

The present disclosure relates to nucleic acid molecules that can be suitable for use as vaccine vectors.

Lipid nanoparticles (LNPs) may be used as a platform for vaccine vector delivery. LNPs may comprise ionizable cationic lipids, cholesterol, phospholipids (such as distearoylphosphatidylcholine), and polyethylene glycol (PEG)-lipid. lonizable cationic lipids participate in nanoparticle packaging by interacting with negatively charged RNA molecules. Upon administration, LNPs are rapidly cleared from injected tissues, and are therefore less likely to induce inflammation and tissue damage.

Thus, in one instance of the present disclosure, the nucleic acid molecules as described anywhere herein are packaged into a delivery system. In one instance the delivery system is an LNP. Thus, the present disclosure also relates to LNPs comprising a nucleic acid molecule as described anywhere herein.

In one instance, the LNPs comprise nucleic acid molecules as described anywhere herein, wherein the nucleic acid molecule encodes an antigenic protein. In one instance, the LNPs comprise one or more nucleic acid molecules as described anywhere herein, wherein the molecules encode a CoV S protein.

### Uses

The present disclosure provides for a nucleic acid molecule described herein for use in medicine.

In one instance, a nucleic acid molecule of the disclosure encoding a therapeutic protein or peptide is useful for treating a disease or condition characterised by a lack of said therapeutic protein or peptide.

In one instance, a nucleic acid molecule of the disclosure encoding a disease-associated antigen is useful as a nucleic acid vaccine vector.

Following patient administration, the coding sequence will be transcribed and translated, in the case of a DNA sequence, and translated in the case of an RNA sequence, into the antigenic protein or fragment of an antigenic protein that it encodes for. The production of these antigenic proteins or antigenic protein fragments will stimulate an immune response, leading to the production of neutralising antibodies. Upon infection by a corresponding infectious agent, the presence of neutralising antibodies and memory B cells will increase the speed of the immune response, minimising the severity and length of symptom onset.

The vaccine vectors of the present disclosure may be used as a preventative therapy against a target antigen that causes disease. In one instance of the present disclosure, the vaccine vectors may be used in the prevention of CoV and, in particular, SARS-COV-2.

The vaccine vectors of the present disclosure may also be used as a treatment against a target antigen that has infected a subject. In one instance of the present disclosure, the vaccine vectors may be used in the treatment of CoV and, in particular, SARS-COV-2.

The present disclosure further provides a method of the prevention or treatment of a disease or condition comprising administering a nucleic acid molecule as described anywhere herein to a patient in need thereof. The present disclosure also relates to a nucleic acid molecule as described anywhere herein for use in a method of manufacture of a medicament useful in the prevention or treatment of a disease. In one instance, the disease is caused by CoV. In one instance, the disease is COVID-19.

Further, the present disclosure relates to a method of inducing an immune response in a subject, the method comprising administering a nucleic acid molecule, combination, composition, pharmaceutical composition or formulation as described anywhere herein to the subject.

In one instance, the subject is human.

### EXAMPLES

Here we describe 5′-UTR and 3′-UTR sequences that enhance expression of coding sequences, including in the context of mRNA vaccine vectors.

The following examples further illustrate the disclosure but should not be construed as in any way limiting its scope.

### Example 1: Modification of the 5′-UTR can Affect eGFP mRNA Expression in A549 Cells and HeLa Cells

### Methods:

### UTR Cloning:

5′-UTR candidate sequences (shown in Table 2 below) were cloned with eGFP and a reference 3′-UTR sequence (albumin, as used in a CureVac vector described, for example, in EP2831240). The cloning was either done using restriction sites (RE) or seamless. The 5′ end of the 5′-UTR contained T7 promoter sequence.

### Generation of in Vitro Transcription (IVT) Template Using PCR:

The template for IVT were generated by PCR using Phusion PCR mix (NEB). The upstream primers contain T7 promoter sequence and the downstream primer contained reverse compliment of end of 3′-UTR of respective clone, along with T80 sequence. The resulting PCR product contain sequence encoding relevant sequence in following order: T7 promoter—5′-UTR-eGFP-3′-UTR-A80. The PCR reaction was then treated with DPNI to digest template DNA and purified using PCR purification kit.

mRNA Synthesis Using in Vitro Transcription (IVT):

The template generated for IVT using PCR were used to prepare mRNA using NEB IVT kits that uses T7 RNA polymerase. The protocol was modified to include CleanCap® (AG). The T7 polymerase incorporates CleanCap® (AG) at the start of each mRNA, giving a cap 1 structure at the 5′ end. The mRNA was either produced using unmodified nucleotide or using modified uridine (5′methoxy uridine) in 25% ratio with unmodified uridine. After the reaction completes, the DNA-template was digested using RNase free DNase. mRNA transcripts contained a 5′-CAP-1-5′-UTR-eGFP coding sequence—3′-UTR-A80. mRNA was then purified using silica column and resuspended in water.

### Cell Transfection

Lung A549 cells were grown in T175 flasks in A549 complete medium (Ham's F-12K supplemented with 10% FBS) at 37° C. 5% CO2. Cells were harvested using accutase for 5 min 37° C., then counted, washed and replated. 100,000 cells in 100 μl media were seeded in each well (96W plates) the day before. On the day of transfection, old media was aspirated and 140 μl of media was added to each well. 1 μl of 100 ng/μl of mRNA was diluted in 4 μl of OptiMEM, and 0.3 μl Lipofectamine 2000 was diluted in 4.7 μl OptiMEM. Then mRNA and Lipofectamine complex were mixed by vortexing and spun down before incubating for 5-10 min at RT. 10 μl of mRNA and Lipofectamine mix was added to each well.

Hela cells were grown in T175 flasks in HeLa complete medium (Minimum Essential Medium MEM, supplemented with 10% FBS and 1% non-essential amino acids) at 37° C. 5% CO2. Cells were harvested using accutase for 5 min 37° C., then counted, washed and replated in collagen treated plates. 100,000 cells in 100 μl media were seeded in each well (96W plates, collagen treated) the day before transfection. On the day of transfection, old media was aspirated and 140 μl of media was added to each well. 1 μl of 100 ng/μl of mRNA was diluted in 4 μl of OptiMEM, and 0.3 μl Lipofectamine 2000 was diluted in 4.7 μl OptiMEM. Then mRNA and Lipofectamine complex were mixed by vortexing and spun down before incubating for 5-10 min at RT. 10 μl of mRNA and Lipofectamine mix was added to each well.

Quantification of eGFP Expression:

eGFP fluorescence was detected using incucyte machine that captures images from the live cells. The fluorescence was measured from the images using Incucyte software in relative fluorescence units (RFU). The data reported in the Figures shows eGFP fluorescence 24 h post-transfection.

### Results:

Table 2 shows expression levels by the candidate 5′-UTRs. Sequences for candidate 5′-UTRs are shown in SEQ ID Nos:1-8.

TABLE 2

| | | eGFP expression | eGFP expression |
|---|---|---|---|
| ID | | fold-increase vs | fold-increase vs |
| Number | 5′-UTR | control in A549 cells | control in Hela cells |
| 11 | GOT1 | 4 | 13 |
| 37 | PRKACB | 3 | 13 |
| 52 | Modified (m) | 3 | 7 |
| | CHIT1 | | |
| 53 | GUSB | 3 | 14 |

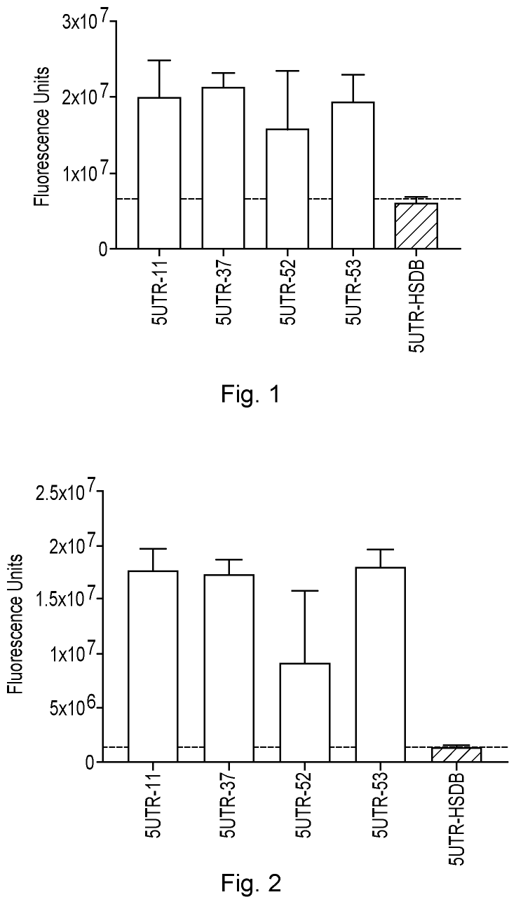

The inclusion of a candidate 5′-UTR (Table 2) was shown to increase the expression of eGFP encoding mRNAs in A549 cells, when compared to eGFP expression with both a control 5′-UTR (HSD17B4) and control 3′-UTR (albumin) (FIG. 1). Inclusion of the GOT1, PRKACB, CHIT1 or GUSB 5′-UTRs corresponded to a 4, 3, 3 and 3-fold increase in eGFP expression, respectively, when compared to expression with a control 5′-UTR (HSD17B4).

This result was substantiated in Hela cells, in which replacement of the control 5′-UTR with a candidate 5′-UTR (Table 2) was shown to increase eGFP-encoding mRNA expression (FIG. 2). Inclusion of the GOT1, PRKACB, CHIT1 or GUSB 5′-UTRs corresponded to a 13, 13, 7 and 14-fold increase in eGFP expression, respectively, when compared to expression with a control 5′-UTR (HSD17B4).

### Example 2: Modification of the 3′-UTR can Affect eGFP mRNA Expression in A549 Cells and HeLa Cells

Methods:

### UTR Cloning:

3′-UTR candidate sequences (shown in Table 3 below) were cloned with eGFP and a reference 5′-UTR sequence (HSD17B4, as used in a CureVac vector, described, for example, in EP2831240). The cloning was either done using restriction sites (RE) or seamless. The 5′ end of 5′-UTR contained T7 promoter sequence.

### Generation of in Vitro Transcription (IVT) Template Using PCR:

The template for IVT were generated by PCR using Phusion PCR mix (NEB). The upstream primers contain T7 promoter sequence and the downstream primer contained reverse compliment of end of 3′-UTR of respective clone, along with T80 sequence. The resulting PCR product contain sequence encoding relevant sequence in following order: T7 promoter—5′-UTR-eGFP-3′-UTR-A80. The PCR reaction was then treated with DPNI to digest template DNA and purified using PCR purification kit.

mRNA Synthesis Using in Vitro Transcription (IVT):

The template generated for IVT using PCR were used to prepare mRNA using NEB IVT kits that uses T7 RNA polymerase. The protocol was modified to include CleanCap® (AG). The T7 polymerase incorporates CleanCap® (AG) at the start of each mRNA, giving cap 1 structure at the 5′ end. The mRNA was either produced using unmodified nucleotide or using modified uridine (5′methoxy uridine) in 25% ratio with unmodified uridine. After the reaction completes, the DNA-template was digested using RNase free DNase. mRNA transcripts contained a 5′-CAP-1-5′-UTR-eGFP-3′-UTR-A80. mRNA was then purified using silica column and resuspended in water.

### Cell Transfection

Lung A549 cells were grown in T175 flasks in A549 complete medium (Ham's F-12K supplemented with 10% FBS) at 37° C. 5% CO2. Cells were harvested using accutase for 5 min 37° C., then counted, washed and replated. 100,000 cells in 100 μl media were seeded in each well (96W plates) the day before. On the day of transfection, old media was aspirated and 140 μl of media was added to each well. 1 μl of 100 ng/μl of mRNA was diluted in 4 μl of OptiMEM, and 0.3 μl Lipofectamine 2000 was diluted in 4.7 μl OptiMEM. Then mRNA and Lipofectamine complex were mixed by vortexing and spun down before incubating for 5-10 min at RT. 10 μl of mRNA and Lipofectamine mix was added to each well.

HeLa cells were grown in T175 flasks in HeLa complete medium (Minimum Essential Medium MEM, supplemented with 10% FBS and 1% non-essential amino acids) at 37° C. 5% CO2. Cells were harvested using accutase for 5 min 37° C., then counted, washed and replated in collagen treated plates. 100,000 cells in 100 μl media were seeded in each well (96W plates, collagen treated) the day before transfection. On the day of transfection, old media was aspirated and 140 μl of media was added to each well. 1 μl of 100 ng/μl of mRNA was diluted in 4 μl of OptiMEM, and 0.3 μl Lipofectamine 2000 was diluted in 4.7 μl OptiMEM. Then mRNA and Lipofectamine complex were mixed by vortexing and spun down before incubating for 5-10 min at RT. 10 μl of mRNA and Lipofectamine mix was added to each well. 1

Quantification of eGFP Expression:

eGFP fluorescence was detected using incucyte machine that captures images from the live cells. The fluorescence was measured from the images using Incucyte software in relative fluorescence units (RFU). The data reported in the Figures shows eGFP fluorescence 24 h post-transfection.

### Results:

Table 3 shows expression levels by the candidate 3′-UTRs. Sequences for candidate 3′-UTRs are shown in SEQ ID NO NOs:9 to 14.

TABLE 3

| | | eGFP expression | eGFP expression |
|---|---|---|---|
| ID | | fold-increase vs | fold-increase vs |
| Number | 3′-UTR | control in A549 cells | control in Hela cells |
| 3 | CHIT1 | 3 | 7 |
| 4 | CS | 3 | 4 |
| 36 | PKLR | 2 | 3 |

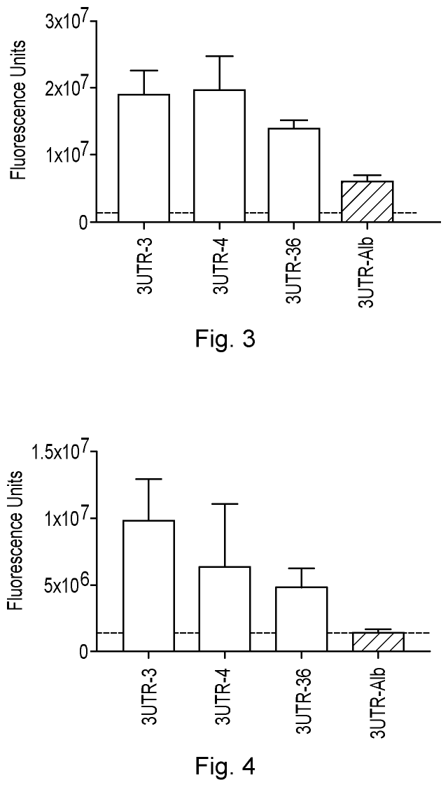

The addition of a candidate 3′-UTR (Table 3) was shown to increase the expression of eGFP encoding mRNAs in A549 cells, when compared to eGFP expression with both a control 3′-UTR (albumin) and control 3′-UTR (HSD17B4) (FIG. 3). The addition of the CHIT1, CS or PKLR 3′-UTR resulted in a 3, 3, and 2-fold increase in eGFP expression, respectively, when compared to expression with a control 3′-UTR (albumin).

This result was replicated in Hela cells, in which replacement of the control 3′-UTR (albumin) with a candidate 3′-UTR (Table 3) was shown to increase eGFP encoding mRNA expression (FIG. 4). As such, addition of the, CHIT1, CS or PKLR 3′-UTRs corresponded with a 7, 4 and 3-fold increase in eGFP expression, respectively, when compared to expression with a control 3′-UTR (albumin).

### Example 3: Combining a Modified 5′-UTR with a Modified 3′-UTR can Increase eGFP mRNA Expression

A combination of selected 5′-UTRs (Table 2) along with selected 3′-UTRs (Table 3) were assessed, to determine whether a combinatory approach could further increase eGFP mRNA expression. The following combinations were assayed: GOT1/CHIT1, GOT1/CS, PRKACB/CHIT1, PRKACB/CS, CHIT1/CHIT1 and CHIT1/CS.

### Methods:

### UTR Cloning:

The 5′-UTRs and 3′-UTRs were cloned with eGFP as the ORF. The cloning was either done using restriction sites (RE) or seamless. The 5′ end of 5′-UTR contained T7 promoter sequence.

### Generation of in Vitro Transcription (IVT) Template Using PCR:

The template for IVT were generated by PCR using Phusion PCR mix (NEB). The upstream primers contain T7 promoter sequence and the downstream primer contained reverse compliment of end of 3′-UTR of respective clone, along with T80 sequence. The resulting PCR product contain sequence encoding relevant sequence in following order: T7 promoter—5′-UTR-ORF-3′-UTR-A80. The PCR reaction was then treated with DPNI to digest template DNA and purified using PCR purification kit.

mRNA Synthesis Using in Vitro Transcription (IVT):

The template generated for IVT using PCR were used to prepare mRNA using NEB IVT kits that uses T7 RNA polymerase. The protocol was modified to include CleanCap® (AG). The T7 polymerase incorporates CleanCap® (AG) at the start of each mRNA, giving cap 1 structure at the 5′ end. The mRNA was either produced using unmodified nucleotide or using modified uridine (5′methoxy Uridine) in 25% ratio with unmodified uridine. After the reaction completes, the DNA-template was digested using RNase free DNase. mRNA transcripts contained a 5′-CAP-1-5′-UTR-ORF-3′-UTR-A80. mRNA was then purified using silica column and resuspended in water.

### Cell Transfection:

Lung A549 cells were grown in T175 flasks in A549 complete medium (Ham's F-12K supplemented with 10% FBS) at 37° C. 5% CO2. Cells were harvested using accutase for 5 min 37° C., then counted, washed and replated. 100,000 cells in 100 μl media were seeded in each well (96W plates) the day before. On the day of transfection, old media was aspirated and 140 μl of media was added to each well. 1 μl of 100 ng/μl of mRNA was diluted in 4 μl of OptiMEM, and 0.3 μl Lipofectamine 2000 was diluted in 4.7 μl OptiMEM. Then mRNA and Lipofectamine complex were mixed by vortexing and spun down before incubating for 5-10 min at RT. 10 μl of mRNA and Lipofectamine mix was added to each well.

Quantification of eGFP Expression:

eGFP fluorescence was detected using incucyte machine that captures images from the live cells. The fluorescence was measured from the images using Incucyte software in relative fluorescence units (RFU). The data reported in the Figures shows eGFP fluorescence 24 h post-transfection.

### Results:

It was previously shown that the incorporation of selected 5′-UTRs and 3′-UTRs with control 3′-UTRs and 5′-UTRs, respectively, could increase the expression of an eGFP (see Examples 1 and 2).

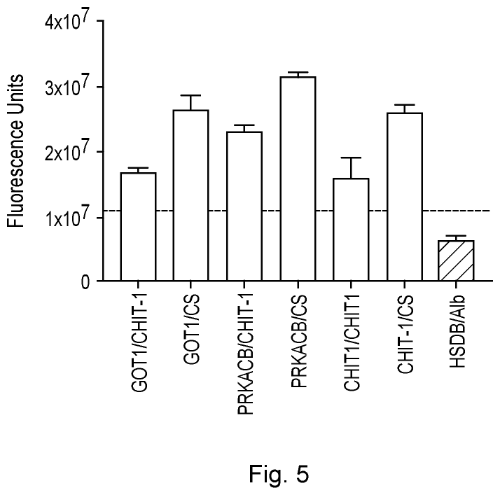

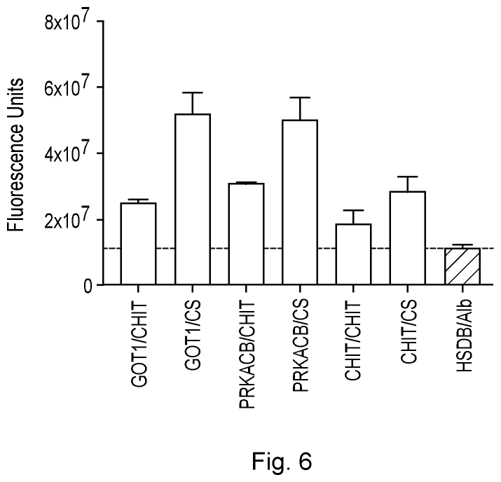

The eGFP mRNA expression with combinations of these 5′-UTRs and 3′-UTRs were compared to expression with a control 5′-UTR (HSD17B4) and a 3′-UTR (albumin). In all instances, eGFP mRNA expression with a combination of a candidate 5′-UTR and a candidate 3′-UTR was increased, in comparison to control in A549 cells (FIG. 5 and FIG. 6). Interestingly, the general trend for each combination was conserved irrespective of whether the mRNA comprised modified base (FIG. 6) or not (FIG. 5). In both instances, the GOT/CS, PRKACB/CS and CHIT/CS resulted in the greatest increase in GFP fluorescence compared to the control mRNA (HSDB/Alb). Surprisingly, GOT/CS1 appeared to work best in a modified mRNA context (FIG. 6), whereas PRKACB/CS resulted in highest GFP fluorescence levels when wild-type bases were used.

### Example 4: Combining a Modified 5′-UTR with a Modified 3′-UTR can Increase the Expression of scFv-Fc Encoding mRNA

In order to study whether the combination of UTRs work as well for increasing expression levels independent of the gene of interest, the combinations were tested for scFv expression. This was again performed in both wild-type mRNA and modified mRNA.

### Methods:

### UTR Cloning:

The 5′-UTRs and 3′-UTRs were cloned with scFv-Fc encoding mRNA. The cloning was either done using restriction sites (RE) or seamless. The 5′ end of 5′-UTR contained T7 promoter sequence.

### Generation of in Vitro Transcription (IVT) Template Using PCR:

The template for IVT were generated by PCR using Phusion PCR mix (NEB). The upstream primers contain T7 promoter sequence and the downstream primer contained reverse compliment of end of 3′-UTR of respective clone, along with T80 sequence. The resulting PCR product contain sequence encoding relevant sequence in following order: T7 promoter—5′-UTR-ORF-3′-UTR-A80. The PCR reaction was then treated with DPNI to digest template DNA and purified using PCR purification kit.

mRNA Synthesis Using in Vitro Transcription (IVT):

The template generated for IVT using PCR were used to prepare mRNA using NEB IVT kits that uses T7 RNA polymerase. The protocol was modified to include CleanCap® (AG). The T7 polymerase incorporates CleanCap® (AG) at the start of each mRNA, giving cap 1 structure at the 5′ end. The mRNA was either produced using unmodified nucleotide or using modified uridine (5′methoxy uridine) in 25% ratio with unmodified uridine. After the reaction completes, the DNA-template was digested using RNase free DNase. mRNA transcripts contained a 5′-CAP-1-5′-UTR-ORF-3′-UTR-A80. mRNA was then purified using silica column and resuspended in water.

### Cell Transfection

Lung A549 cells were grown in T175 flasks in A549 complete medium (Ham's F-12K supplemented with 10% FBS) at 37° C. 5% CO2. Cells were harvested using accutase for 5 min 37° C., then counted, washed and replated. 100,000 cells in 100 μl media were seeded in each well (96W plates) the day before. On the day of transfection, old media was aspirated and 140 μl of media was added to each well. 1 μl of 100 ng/μl of mRNA was diluted in 4 μl of OptiMEM, and 0.3 μl Lipofectamine 2000 was diluted in 4.7 μl OptiMEM. Then mRNA and Lipofectamine complex were mixed by vortexing and spun down before incubating for 5-10 min at RT. 10 μl of mRNA and Lipofectamine mix was added to each well. 100 μl supernatant was collected at specified timepoints.

scFv-Fc Quantification:

Cell supernatant (sup) was harvested from the cells transfected with scFv-Fc mRNA after 24 hours. The sups were frozen at −80 C until quantified. For quantification, cis-bio kit for Fc quantification was used. The principle for quantification is based on competitive immunoassay using HTRF technology. hFc-tagged proteins (or antibody) can displace the binding between IgG labelled with d2 and PAb anti-human Fc labelled with Cryptate. Specific signal (i.e. energy transfer) is inversely proportional to the concentration of human Fc in the sample or standard. A standard curve from known concentrations of scFv-Fc is produced and the signal from sups of scFv-Fc transfected cells was interpolated using this standard curve to quantify the amount of scFv-Fc present in the sup. The concentration of scFv-Fc was measured in ng/ml. The data shown in the Figures reports scFv-Fc levels 24 h post transfection.

### Results:

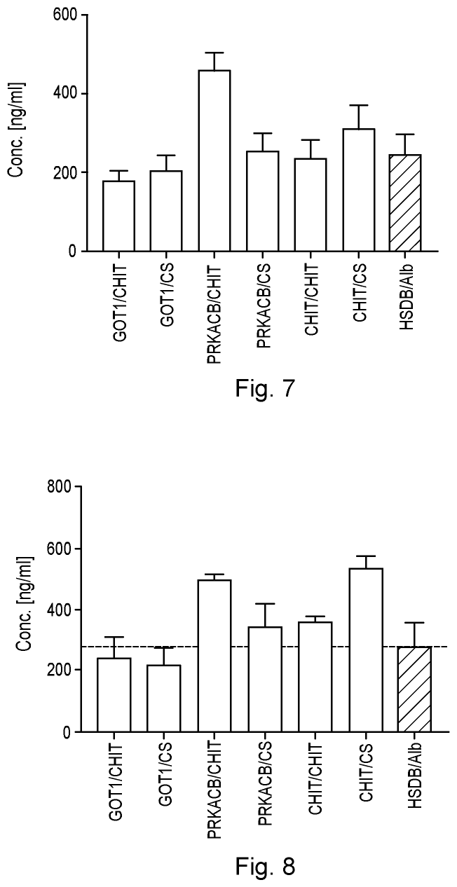

Once again, the scFv mRNA expression with combinations of these 5′-UTRs and 3′-UTRs were compared to expression with a control 5′-UTR (HSD17B4) and a 3′-UTR (albumin). Interestingly, as with eGFP fluorescence levels in Example 3, the PRKACB/CHIT resulted in the highest expression levels when wild-type mRNA was used (FIG. 7). However, apart from CHIT/CS, most other of the UTR combos did not impact detectable scFv expression levels compared to the control (HSBD/Alb). By contrast, CHIT/CS and PRKACB/CHIT performed best compared to the control when modified mRNA was used as the expression substrate (FIG. 8).

Therefore, out of all novel UTRs tested, whether alone or in combination, the CHIT/CS and PRKACB/CHIT combinations generated the greatest increase in expression levels, agnostic of the gene of interest or whether the mRNA comprised modified or wild-type U.

### Example 5-Enhanced in Vitro Protein Expression From mRNA Vaccine Vectors

Next, one of the optimum UTR combinations (CHIT/CS) was tested head-to-head against mRNA comprising putative UTR pairs from Moderna and Pfizer/BioNTech (mRNA Comp A and mRNA Comp B) using the UTR sequences reported on 14 Apr. 2021 by Andrew Fire and colleagues at Stanford University via GitHub. This time, modified mRNA comprised 100%5′methoxy uridine (rather than 25%, as in Examples 1-4).

### EGFP Construct Cloning:

mRNA constructs encoding the EGFP reporter and employing the CHIT1 5′-UTR (SEQ ID NO: 19) paired with a CS 3′-UTR (SEQ ID NO:21) were designed (mRNA_AZ). In parallel, mRNA comparator A and mRNA comparator B constructs were designed to encode EGFP flanked by the putative UTR sequences from each of the COVID vaccines mentioned above. The cloning was performed by Gibson-based assembly methods. Each plasmid possessed a T7 promoter sequence upstream of each 5′ UTR and a poly A track of 80 basepairs long downstream of the 3′ UTR with a single BspQI site for subsequent linearization. All EGFP coding sequences were identical. The full 5′-UTR and 3′-UTR sequences comprising sequences derived from CHIT1 and CS UTRs are shown below (SEQ ID Nos: 19 and 21, respectively).

### Generation of in Vitro Transcription (IVT) Template:

The template for IVT were generated following plasmid purification from bacterial cells in a manner similar to as outlined in Examples 1-4.

mRNA Synthesis Using in Vitro Transcription (IVT):

The template generated for IVT was used to prepare mRNA using NEB IVT kits employing T7 RNA polymerase. The protocol was modified to include CleanCap® (AG). The T7 polymerase incorporates CleanCap® (AG) at the start of each mRNA, giving a cap 1 structure at the 5′ end. The mRNA was either produced using unmodified nucleotide or using modified uridine (N1-methylpseudouridine) at 100%. Both mRNA comp A and mRNA comp B (the mRNAs comprising the putative UTRs derived from the GitHub database) contained 100% modified U. After the reaction completed, the DNA template was digested using RNase-free DNase. mRNA was then purified using silica column and resuspended in water.

### Quantification of EGFP Expression:

Purified mRNA was transfected into either BHK-21 or HEK293 cells with Lipofectamine MessengerMAX transfection reagent (ThermoFisher) per manufacturer's protocol. EGFP fluorescence was detected using an IncuCyte instrument which captures images from live, RNA-transfected cells over the course of 96 hours. The fluorescence was measured from the images using IncuCyte software as relative fluorescence units (arbitrary units).

### Results:

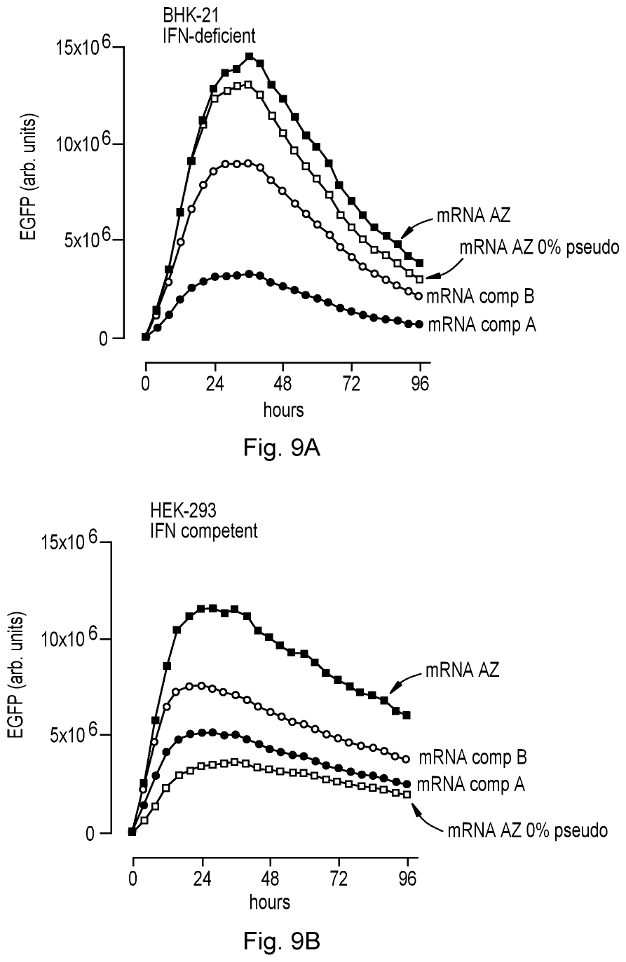

The mRNAs employing the 5′-UTR CHIT1 (SEQ ID NO: 19) and 3′-UTR CS (SEQ ID NO: 21) UTR set generated the highest levels of EGFP expression in both BHK-21 (FIG. 9A) and HEK293 (FIG. 9B) cells compared to two competitor mRNA molecules (mRNA Comp A and mRNA Comp B). Maximal expression from mRNA was dependent on the incorporation of modified nucleotides like N1-Methylpseudouridine (pseudoU) in order to evade host cell anti-viral responses among the HEK293 cells (FIG. 9B).

### Example 6—Efficacy of mRNA Vaccine Vectors Comprising Antigen-Linker-Ferritin Sequences

The overall purpose of this study was to determine the immunogenicity of a candidate SARS-CoV-2 mRNA vaccine comprising the mCHIT/CS UTR combination in mice and naïve non-human primates. The mRNA vaccine encoded a stabilised Spike (S) protein-ferritin subunit fusion protein that, when expressed, assembles into a nanoparticle for high concentration antigen display.

### Methods

### Mouse Study

Groups of naïve BALB/c mice (n=6 per group) were administered LNP-formulated mRNA vaccines twice (21 days apart) as 50 μl injections intramuscularly in the thigh muscle. Fourteen days after the second vaccination, mice were bled and sera obtained to perform SARS-COV-2 pseudovirus-based neutralization assays. Both the magnitude and breadth of neutralizing antibody (nAb) responses among the different groups were evaluated using a panel of pseudoviruses bearing the following SARS-COV-2 Spike proteins of interest: Delta, Wuhan (D614G), BA.1, BA.2 and BA.4/5. Sigmoidal curves, taking averages of triplicates at each serum dilution, were generated, and 50% (ID₅₀) neutralizing activity was calculated, considering uninfected cells to represent 100% neutralization and cells transduced with only virus to represent 0% neutralization.

### Non-Human Primate Studies

Groups of naïve cynomolgus non-human primates (NHPs; n=6 per group) were administered LNP-formulated mRNA vaccines twice (28 days apart) as 1 ml injections intramuscularly. Each animal received a 10 μg dose per vaccination. Fourteen days after the second vaccination, NHPs were bled and sera obtained to perform SARS-COV-2 pseudovirus-based neutralization assays. Both the magnitude and breadth of neutralizing antibody (nAb) responses among the different groups were evaluated using a panel of pseudoviruses bearing the following SARS-CoV-2 Spike proteins of interest: Delta, Wuhan (D614G), BA.1, BA.2 and BA.4/5. Sigmoidal curves, taking averages of triplicates at each serum dilution, were generated, and 50% (ID₅₀) neutralizing activity was calculated, considering uninfected cells to represent 100% neutralization and cells transduced with only virus to represent 0% neutralization.

In both the mouse and NHP studies, groups were administered either a Delta S protein ferritin construct (Delta FL VLP) or a Delta S protein only-encoding mRNA (Delta FL Spike). This enabled comparison of the immunogenicity and reactogenicity profiles of the mRNA-based nanoparticle vaccine approach compared to the Spike-only approach used in tozinameran and elasomeran. Both delta FL VLP and Delta FL Spike constructs comprised the CHIT1/CS UTR combination disclosed herein.

### Results

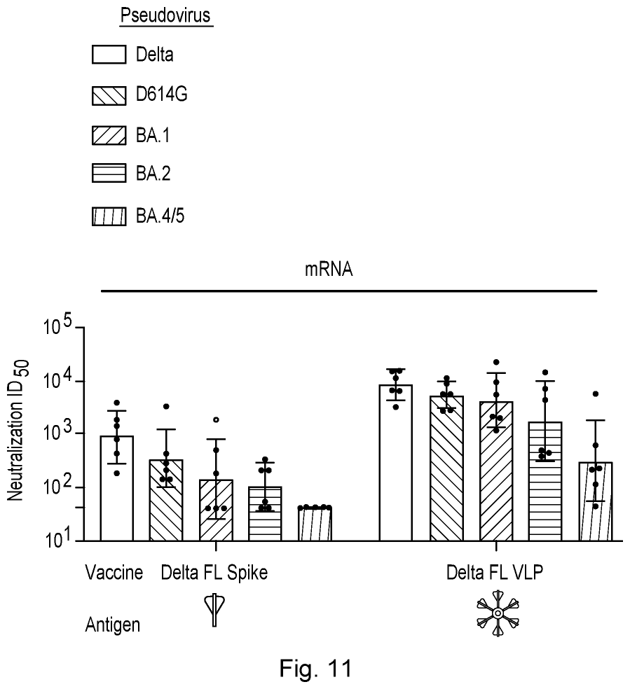

The results, shown in FIGS. 10 (mice) and 11 (NHPs), show that the amplitude of the neutralising antibody response against the cognate variant (Delta) is significantly higher in the Delta FL VLP inoculated animals compared to those that received Delta FL Spike. Interestingly, the breadth of the neutralising antibody response is also greater for all variants tested (FIGS. 10 and 11).

Table 4 below demonstrates the mRNA VLP/FL spike fold change for the data in FIG. 11.

TABLE 4

| | mRNA VLP/FL | mRNA VLP/FL |
|---|---|---|
| Virus | spike fold-change¹ | spike p value² |
| Delta | 9 | 0.002 |
| D614G | 15 | <0.001 |
| BA.1 | 29 | <0.001 |
| BA.2 | 18 | <0.001 |
| BA. 4/5 | >8 | <0.001 |

¹Fold change = mRNA VLP geometric mean titre (GMT)/mRNA FL spike GMT
²p-values calculated using two-sided t-test

This suggests that broadly neutralising responses are obtainable from a single vaccine construct when mRNA is used to launch a nanoparticle antigen. This may lead to better efficacy against multiple existing, and possibly future, variants of concern, without the requirement for boosters to be independently generated each time a novel dominant variant emerges.

Sequences

5′-UTR modified (m) CHIT1
DNA Sequence (SEQ ID 1):
ATTGTGCTGCATC
RNA Sequence (SEQ ID 2):
AUUGUGCUGCAUC
5′-UTR PRKACB
DNA Sequence (SEQ ID 3):
ATTCTGCTGTTTGCTCCTTGCCAGGTTCAAC
RNA Sequence (SEQ ID 4):
AUUCUGCUGUUUGCUCCUUGCCAGGUUCAAC
5′-UTR GOT1
DNA Sequence (SEQ ID 5):
AAAATCTCTTGATTCCTAGTCTCTCGAT
RNA Sequence (SEQ ID 6):
AAAAUCUCUUGAUUCCUAGUCUCUCGAU
5′-UTR GUSB
DNA Sequence (SEQ ID 7):
ATCCTCAACCAAGCGCCGCAGACGGTGGCCGAGCGGGGGACCGGGAAGC
RNA Sequence (SEQ ID 8):
AUCCUCAACCAAGCGCCGCAGACGGUGGCCGAGCGGGGGACCGGGAAGC
3′-UTR CS
DNA Sequence (SEQ ID 9):
AACTGGAGACTGGGTGAAAGTGACTACCAGAAAGTGAGGAAGCCTAAATAAA
RNA Sequence (SEQ ID 10):
AACUGGAGACUGGGUGAAAGUGACUACCAGAAAGUGAGGAAGCCUAAAUAAA
3′-UTR CHIT1
DNA Sequence (SEQ ID 11):
GTCGCTAAAGCCCCTCCAGTCCCAGCTTTGAGGCTGGGCCCAGGATCACTCTACAGCC
TGCCTCCTGGGTTTTCCCTGGGGGCCGCAATCTGGCTCCTGCAGGCCTTTCTGTGGTC
TTCCTTTATCCAGGCTTTCTGCTCTCAGCCTTGCCTTCCTTTTTTCTGGGTCTCCTGGGC
TGCCCCTTTCACTTGCAAAATAAA
RNA Sequence (SEQ ID 12):
GUCGCUAAAGCCCCUCCAGUCCCAGCUUUGAGGCUGGGCCCAGGAUCACUCUACAG
CCUGCCUCCUGGGUUUUCCCUGGGGGCCGCAAUCUGGCUCCUGCAGGCCUUUCUG
UGGUCUUCCUUUAUCCAGGCUUUCUGCUCUCAGCCUUGCCUUCCUUUUUUCUGCGA
CUCCUGGG CUGCCCCUUUCACUUGCAAAAUAAA
3′-UTR PKLR
DNA Sequence (SEQ ID 13):
GACGCCCCTCCCTCCTCTGGAGTCTACGTTCTCCAGCCCACACCCCTCCAAAGCCCCA
CCTTTAAGTCCTCTCTTCTCTATTCCTGACCCTCCCTACCTGAGGCCTATCTGAGACTAT
AACTGTCATCTAGCCCCTTCGAGGTTGCCCCTTCCCCATCTCCATTTCACACAGGTCCT
GAAAGTCTGTGTCCAATTATGCACTGGCCACCCAACAGCACCAATTGTACATTCCCTGC
ATCCAATCTGCTCAGCAGGCCCTAAGATGCCTTGAGTCTTTAATCCCA
RNA Sequence (SEQ ID 14):
GACGCCCCUCCCUCCUCUGGAGUCUACGUUCUCCAGCCCACACCCCUCCAAAGCCC
CACCUUUAAGUCCUCUCUUCUCUAUUCCUGACCCUCCCUACCUGAGGCCUAUCUGA
GACUAUAACUGUCAUCUAGCCCCUUCGAGGUUGCCCCUUCCCCAUCUCCAUUUCACA
CAGGUCCUGAAAGUCUGUGUCCAAUUAUGCACUGGCCACCCAACAGCACCAAUUGUA
CAUUCCCUGCAUCCAAUCUGCUCAGCAGGCCCUAAGAUGCCUUGAGUCUUUAAUCCC
A
T7 Promoter Sequence (SEQ ID 15):
TAATACGACTCACTATAAGG
3′ UTR Albumin Sequence (SEQ ID 16):
GCATCACATTTAAAAGCATCTCAGCCTACCATGAGAATAAGAGAAAGAAAATGAAGATC
AATAGCTTATTCATCTCTTTTTCTTTTTCGTTGGTGTAAAGCCAACACCCTGTCTAAAAAA
CATAAATTTCTTTAATCATTTTGCCTCTTTTCTCTGTGCTTCAATTAATAAAAAATGGAAA
GAACCT
5′ UTR HSDB Sequence (SEQ ID 17):
GTCCCGCAGTCGGCGTCCAGCGGCTCTGCTTGTTCGTGTGTGTGTCGTTGCAGGCCTT
ATT
5′-UTR CHIT1 with additional sequence elements (mCHIT1 5′-UTR sequence underlined)
DNA Sequence (SEQ ID 18):
AGGATTGTGCTGCATCAAGCTTGCCGCCACC
RNA Sequence (SEQ ID 19):
AGGAUUGUGCUGCAUCAAGCUUGCCGCCACC
3′-UTR CS with additional sequence elements (CS 3′-UTR sequence underlined)
DNA Sequence (SEQ ID 20):
TGATGATAATAGGACTAGTGGATCCAACTGGAGACTGGGTGAAAGTGACTACCAGAAA
GTGAGGAAGCCTAAATAAACCTAGCGTACGTAAAAAATGGAAAGAACCTAGCGTACG
RNA Sequence (SEQ ID 21):
UGAUGAUAAUAGGACUAGUGGAUCCAACUGGAGACUGGGUGAAAGUGACUACCAGAA
AGUGAGGAAGCCUAAAUAAACCUAGCGUACGUAAAAAAUGGAAAGAACCUAGCGUAC
G
SARS-CoV-2 Wuhan D614G-linker-ferritin DNA sequence (SEQ ID NO: 22)
ATGCCCCTCCTTCTCCTTCTTCCCCTCCTTTGGGCTGGAGCGCTGGCCTCTCAGTGTGT
AAATCTCACCACAAGAACCCAGCTGCCCCCTGCCTATACCAATTCCTTCACACGGGGC
GTGTACTATCCCGACAAGGTGTTTAGATCTAGCGTGCTGCACTCCACACAGGATCTGTT
TCTGCCTTTCTTTTCTAACGTGACCTGGTTCCACGCCATCCACGTGAGCGGCACCAATG
GCACAAAGAGGTTCGACAATCCAGTGCTGCCCTTTAACGATGGCGTGTACTTCGCCTC
CACCGAGAAGTCTAACATCATCCGCGGCTGGATCTTTGGCACCACACTGGACAGCAAG
ACACAGTCCCTGCTGATCGTGAACAATGCCACCAACGTGGTCATCAAGGTGTGCGAGT
TCCAGTTTTGTAATGATCCATTCCTGGGCGTGTACTATCACAAGAACAATAAGTCTTGGA
TGGAGAGCGAGTTTCGGGTGTATTCCTCTGCCAACAATTGCACATTTGAGTACGTGTCC
CAGCCCTTCCTGATGGACCTGGAGGGCAAGCAGGGCAATTTCAAGAACCTGCGGGAG
TTCGTGTTTAAGAATATCGATGGCTACTTCAAGATCTACTCCAAGCACACCCCCATCAAC
CTGGTGCGGGACCTGCCACAGGGCTTCTCTGCCCTGGAGCCACTGGTGGATCTGCCC
ATCGGCATCAACATCACCCGGTTTCAGACACTGCTGGCCCTGCACAGAAGCTACCTGA
CACCAGGCGACAGCTCCTCTGGATGGACCGCAGGAGCAGCAGCCTACTATGTGGGCT
ATCTGCAGCCCCGGACCTTCCTGCTGAAGTACAACGAGAATGGCACCATCACAGACGC
CGTGGATTGCGCCCTGGATCCCCTGTCTGAGACAAAGTGTACACTGAAGAGTTTTACC
GTGGAGAAGGGCATCTATCAGACAAGCAATTTCAGGGTGCAGCCTACCGAGTCCATCG
TGCGCTTTCCCAATATCACAAACCTGTGCCCTTTTGGCGAGGTGTTCAACGCCACCAGA
TTCGCCAGCGTGTACGCCTGGAATAGGAAGCGCATCTCCAACTGCGTGGCCGACTATT
CTGTGCTGTACAACAGCGCCTCCTTCTCTACCTTTAAGTGCTATGGCGTGAGCCCCACA
AAGCTGAATGACCTGTGCTTTACCAACGTGTACGCCGATTCCTTCGTGATCAGGGGCG
ACGAGGTGCGCCAGATCGCACCAGGACAGACAGGCAAGATCGCAGACTACAATTATAA
GCTGCCTGACGATTTCACCGGCTGCGTGATCGCCTGGAACTCTAACAATCTGGATAGC
AAAGTGGGCGGCAACTACAATTATCTGTACCGGCTGTTTAGAAAGTCTAATCTGAAGCC
ATTCGAGCGGGACATCTCCACAGAGATCTACCAGGCCGGCTCTACCCCCTGCAATGGC
GTGGAGGGCTTTAACTGTTATTTCCCTCTGCAGAGCTACGGCTTCCAGCCAACCAACG
GCGTGGGCTATCAGCCCTACAGAGTGGTGGTGCTGTCTTTTGAGCTGCTGCACGCACC
TGCAACAGTGTGCGGACCAAAAAAGAGCACCAATCTGGTGAAGAACAAGTGCGTGAAC
TTCAACTTCAACGGCCTGACCGGAACAGGCGTGCTGACCGAGTCCAACAAGAAGTTCC
TGCCTTTTCAGCAGTTCGGCAGGGACATCGCAGATACCACAGACGCCGTGCGCGACCC
TCAGACCCTGGAGATCCTGGATATCACACCATGCTCCTTCGGCGGCGTGTCTGTGATC
ACACCAGGCACCAATACAAGCAACCAGGTGGCCGTGCTGTATCAGGGCGTGAATTGTA
CCGAGGTGCCAGTGGCAATCCACGCAGATCAGCTGACCCCTACATGGCGGGTGTACT
CTACCGGCAGCAACGTGTTCCAGACAAGAGCCGGATGCCTGATCGGAGCAGAGCACG
TGAACAATAGCTATGAGTGCGACATCCCTATCGGCGCCGGCATCTGTGCCTCCTACCA
GACCCAGACAAACTCCCCAGGGTCTGCATCATCTGTGGCAAGCCAGTCCATCATCGCC
TATACCATGAGCCTGGGCGCCGAGAATTCCGTGGCCTACTCCAACAATTCTATCGCCAT
CCCTACCAACTTCACAATCTCCGTGACCACAGAGATCCTGCCAGTGAGCATGACCAAG
ACATCCGTGGACTGCACAATGTATATCTGTGGCGATTCCACCGAGTGCTCTAACCTGCT
GCTGCAGTACGGCAGCTTTTGTACCCAGCTGAATCGCGCCCTGACAGGAATCGCAGTG
GAGCAGGATAAGAACACACAGGAGGTGTTCGCCCAGGTGAAGCAGATCTACAAGACCC
CACCCATCAAGGACTTTGGCGGCTTCAATTTTTCCCAGATCCTGCCCGATCCTAGCAAG
CCATCCAAGAGGTCTTTTATCGAGGACCTGCTGTTCAACAAGGTGACCCTGGCCGATG
CCGGCTTCATCAAGCAGTATGGCGATTGCCTGGGCGACATCGCAGCCCGCGACCTGAT
CTGTGCCCAGAAGTTTAATGGCCTGACCGTGCTGCCTCCACTGCTGACAGATGAGATG
ATCGCCCAGTACACATCTGCCCTGCTGGCAGGAACCATCACAAGGGGATGGACCTTCG
GCGCAGGAGCCGCCCTGCAGATCCCCTTTGCCATGCAGATGGCCTATCGGTTCAACG
GCATCGGCGTGACCCAGAATGTGCTGTACGAGAACCAGAAGCTGATCGCCAATCAGTT
TAACTCCGCCATCGGCAAGATCCAGGACTCTCTGAGCAGCACAGCAAGCGCCCTGGG
CAAGCTGCAGGATGTGGTGAATCAGAACGCCCAGGCCCTGAATACCCTGGTGAAGCAG
CTGTCTAGCAACTTCGGCGCCATCTCCTCTGTGCTGAATGATATCCTGAGCAGGCTGG
ACCCTCCAGAGGCCGAAGTGCAGATCGATCGGCTGATCACTGGTAGGCTGCAGAGTCT
GCAGACATATGTCACCCAGCAGCTGATCAGAGCAGCCGAGATTCGAGCTTCAGCTAAC
TTAGCTGCCACAAAGATGAGTGAGTGCGTGCTGGGCCAGTCCAAGAGGGTGGATTTCT
GCGGAAAAGGCTATCATCTCATGTCCTTCCCCCAAAGCGCACCCCACGGGGTTGTCTT
CCTGCACGTAACGTATGTGCCCGCTCAGGAGAAGAACTTTACTACTGCCCCCGCCATC
TGCCACGATGGGAAAGCTCACTTCCCGAGAGAAGGAGTATTCGTTTCCAATGGGACGC
ATTGGTTCGTCACCCAGCGGAACTTTTACGAACCACAGATCATTACCACGGATAATACA
TTTGTGAGCGGGAATTGCGATGTGGTGATTGGAATTGTAAATAACACAGTGTACGATCC
ACTGCAGTCAGAATTAGACTCAATTAAGGAGGAGTTGGATAAGATTCATAAAAATGGTT
CAGGTGGATCAGGTGATATAGAAAAACTCCTCAATGAACAAGTAAATAAGGAGATGCAA
AGTTCTAACCTGTACATGAGCATGTCTTCTTGGTGTTACACCCATAGCCTCGATGGAGC
GGGATTGTTCCTTTTTGACCACGCTGCGGAGGAGTATGAGCATGCTAAAAAGCTGATAA
TATTTCTCAACGAGAATAATGTTCCAGTGCAATTGACAAGTATATCCGCCCCTGAGCATA
AGTTTGAAGGGCTCACACAAATTTTCCAAAAGGCATACGAACACGAACAGCACATTAGC
GAGTCTATTAACAACATTGTTGATCATGCAATCAAGTCCAAAGATCACGCCACGTTTAAT
TTCCTCCAGTGGTATGTAGCTGAGCAACATGAGGAAGAAGTGTTGTTTAAGGATATTCT
TGATAAAATTGAACTTATTGGAAATGAGAACCATGGCCTCTATCTTGCGGACCAATACGT
CAAGGGAATTGCCAAGTCCCGCAAGAGT
SARS-CoV-2 Wuhan D614G Spike Protein-linker-ferritin RNA sequence (SEQ ID NO: 23)
AUGCCCCUCCUUCUCCUUCUUCCCCUCCUUUGGGCUGGAGCGCUGGCCUCUCAGUG
UGUAAAUCUCACCACAAGAACCCAGCUGCCCCCUGCCUAUACCAAUUCCUUCACACG
GGGCGUGUACUAUCCCGACAAGGUGUUUAGAUCUAGCGUGCUGCACUCCACACAGG
AUCUGUUUCUGCCUUUCUUUUCUAACGUGACCUGGUUCCACGCCAUCCACGUGAGC
GGCACCAAUGGCACAAAGAGGUUCGACAAUCCAGUGCUGCCCUUUAACGAUGGCGU
GUACUUCGCCUCCACCGAGAAGUCUAACAUCAUCCGCGGCUGGAUCUUUGGCACCA
CACUGGACAGCAAGACACAGUCCCUGCUGAUCGUGAACAAUGCCACCAACGUGGUCA
UCAAGGUGUGCGAGUUCCAGUUUUGUAAUGAUCCAUUCCUGGGCGUGUACUAUCAC
AAGAACAAUAAGUCUUGGAUGGAGAGCGAGUUUCGGGUGUAUUCCUCUGCCAACAA
UUGCACAUUUGAGUACGUGUCCCAGCCCUUCCUGAUGGACCUGGAGGGCAAGCAGG
GCAAUUUCAAGAACCUGCGGGAGUUCGUGUUUAAGAAUAUCGAUGGCUACUUCAAGA
UCUACUCCAAGCACACCCCCAUCAACCUGGUGCGGGACCUGCCACAGGGCUUCUCU
GCCCUGGAGCCACUGGUGGAUCUGCCCAUCGGCAUCAACAUCACCCGGUUUCAGAC
ACUGCUGGCCCUGCACAGAAGCUACCUGACACCAGGCGACAGCUCCUCUGGAUGGA
CCGCAGGAGCAGCAGCCUACUAUGUGGGCUAUCUGCAGCCCCGGACCUUCCUGCUG
AAGUACAACGAGAAUGGCACCAUCACAGACGCCGUGGAUUGCGCCCUGGAUCCCCU
GUCUGAGACAAAGUGUACACUGAAGAGUUUUACCGUGGAGAAGGGCAUCUAUCAGA
CAAGCAAUUUCAGGGUGCAGCCUACCGAGUCCAUCGUGCGCUUUCCCAAUAUCACAA
ACCUGUGCCCUUUUGGCGAGGUGUUCAACGCCACCAGAUUCGCCAGCGUGUACGCC
UGGAAUAGGAAGCGCAUCUCCAACUGCGUGGCCGACUAUUCUGUGCUGUACAACAG
CGCCUCCUUCUCUACCUUUAAGUGCUAUGGCGUGAGCCCCACAAAGCUGAAUGACC
UGUGCUUUACCAACGUGUACGCCGAUUCCUUCGUGAUCAGGGGCGACGAGGUGCGC
CAGAUCGCACCAGGACAGACAGGCAAGAUCGCAGACUACAAUUAUAAGCUGCCUGAC
GAUUUCACCGGCUGCGUGAUCGCCUGGAACUCUAACAAUCUGGAUAGCAAAGUGGG
CGGCAACUACAAUUAUCUGUACCGGCUGUUUAGAAAGUCUAAUCUGAAGCCAUUCGA
GCGGGACAUCUCCACAGAGAUCUACCAGGCCGGCUCUACCCCCUGCAAUGGCGUGG
AGGGCUUUAACUGUUAUUUCCCUCUGCAGAGCUACGGCUUCCAGCCAACCAACGGC
GUGGGCUAUCAGCCCUACAGAGUGGUGGUGCUGUCUUUUGAGCUGCUGCACGCACC
UGCAACAGUGUGCGGACCAAAAAAGAGCACCAAUCUGGUGAAGAACAAGUGCGUGAA
CUUCAACUUCAACGGCCUGACCGGAACAGGCGUGCUGACCGAGUCCAACAAGAAGU
UCCUGCCUUUUCAGCAGUUCGGCAGGGACAUCGCAGAUACCACAGACGCCGUGCGC
GACCCUCAGACCCUGGAGAUCCUGGAUAUCACACCAUGCUCCUUCGGCGGCGUGUC
UGUGAUCACACCAGGCACCAAUACAAGCAACCAGGUGGCCGUGCUGUAUCAGGGCG
UGAAUUGUACCGAGGUGCCAGUGGCAAUCCACGCAGAUCAGCUGACCCCUACAUGG
CGGGUGUACUCUACCGGCAGCAACGUGUUCCAGACAAGAGCCGGAUGCCUGAUCGG
AGCAGAGCACGUGAACAAUAGCUAUGAGUGCGACAUCCCUAUCGGCGCCGGCAUCU
GUGCCUCCUACCAGACCCAGACAAACUCCCCAGGGUCUGCAUCAUCUGUGGCAAGC
CAGUCCAUCAUCGCCUAUACCAUGAGCCUGGGCGCCGAGAAUUCCGUGGCCUACUC
CAACAAUUCUAUCGCCAUCCCUACCAACUUCACAAUCUCCGUGACCACAGAGAUCCU
GCCAGUGAGCAUGACCAAGACAUCCGUGGACUGCACAAUGUAUAUCUGUGGCGAUU
CCACCGAGUGCUCUAACCUGCUGCUGCAGUACGGCAGCUUUUGUACCCAGCUGAAU
CGCGCCCUGACAGGAAUCGCAGUGGAGCAGGAUAAGAACACACAGGAGGUGUUCGC
CCAGGUGAAGCAGAUCUACAAGACCCCACCCAUCAAGGACUUUGGCGGCUUCAAUUU
UUCCCAGAUCCUGCCCGAUCCUAGCAAGCCAUCCAAGAGGUCUUUUAUCGAGGACC
UGCUGUUCAACAAGGUGACCCUGGCCGAUGCCGGCUUCAUCAAGCAGUAUGGCGAU
UGCCUGGGCGACAUCGCAGCCCGCGACCUGAUCUGUGCCCAGAAGUUUAAUGGCCU
GACCGUGCUGCCUCCACUGCUGACAGAUGAGAUGAUCGCCCAGUACACAUCUGCCC
UGCUGGCAGGAACCAUCACAAGCGGAUGGACCUUCGGCGCAGGAGCCGCCCUGCAG
AUCCCCUUUGCCAUGCAGAUGGCCUAUCGGUUCAACGGCAUCGGCGUGACCCAGAA
UGUGCUGUACGAGAACCAGAAGCUGAUCGCCAAUCAGUUUAACUCCGCCAUCGGCAA
GAUCCAGGACUCUCUGAGCAGCACAGCAAGCGCCCUGGGCAAGCUGCAGGAUGUGG
UGAAUCAGAACGCCCAGGCCCUGAAUACCCUGGUGAAGCAGCUGUCUAGCAACUUC
GGCGCCAUCUCCUCUGUGCUGAAUGAUAUCCUGAGCAGGCUGGACCCUCCAGAGGC
CGAAGUGCAGAUCGAUCGGCUGAUCACUGGUAGGCUGCAGAGUCUGCAGACAUAUG
UCACCCAGCAGCUGAUCAGAGCAGCCGAGAUUCGAGCUUCAGCUAACUUAGCUGCC
ACAAAGAUGAGUGAGUGCGUGCUGGGCCAGUCCAAGAGGGUGGAUUUCUGCGGAAA
AGGCUAUCAUCUCAUGUCCUUCCCCCAAAGCGCACCCCACGGGGUUGUCUUCCUGC
ACGUAACGUAUGUGCCCGCUCAGGAGAAGAACUUUACUACUGCCCCCGCCAUCUGC
CACGAUGGGAAAGCUCACUUCCCGAGAGAAGGAGUAUUCGUUUCCAAUGGGACGCA
UUGGUUCGUCACCCAGCGGAACUUUUACGAACCACAGAUCAUUACCACGGAUAAUAC
AUUUGUGAGCGGGAAUUGCGAUGUGGUGAUUGGAAUUGUAAAUAACACAGUGUACG
AUCCACUGCAGUCAGAAUUAGACUCAAUUAAGGAGGAGUUGGAUAAGAUUCAUAAAA
AUGGUUCAGGUGGAUCAGGUGAUAUAGAAAAACUCCUCAAUGAACAAGUAAAUAAGG
AGAUGCAAAGUUCUAACCUGUACAUGAGCAUGUCUUCUUGGUGUUACACCCAUAGCC
UCGAUGGAGCGGGAUUGUUCCUUUUUGACCACGCUGCGGAGGAGUAUGAGCAUGCU
AAAAAGCUGAUAAUAUUUCUCAACGAGAAUAAUGUUCCAGUGCAAUUGACAAGUAUA
UCCGCCCCUGAGCAUAAGUUUGAAGGGCUCACACAAAUUUUCCAAAAGGCAUACGAA
CACGAACAGCACAUUAGCGAGUCUAUUAACAACAUUGUUGAUCAUGCAAUCAAGUCC
AAAGAUCACGCCACGUUUAAUUUCCUCCAGUGGUAUGUAGCUGAGCAACAUGAGGAA
GAAGUGUUGUUUAAGGAUAUUCUUGAUAAAAUUGAACUUAUUGGAAAUGAGAACCAU
GGCCUCUAUCUUGCGGACCAAUACGUCAAGGGAAUUGCCAAGUCCCGCAAGAGU
SARS-CoV-2 Delta-linker-ferritin DNA sequence (SEQ ID NO: 24)
ATGCCCCTCCTTCTCCTTCTTCCCCTCCTTTGGGCTGGAGCGCTGGCCTCTCAGTGTGT
AAATCTCCGTACCCGAACACAACTCCCCCCCGCCTATACCAACAGCTTCACACGCGGA
GTTTACTACCCTGACAAGGTATTCAGGTCCAGTGTACTCCATTCAACACAGGATCTGTT
CCTCCCTTTTTTCAGTAATGTGACCTGGTTTCACGCTATCCACGTAAGCGGCACGAATG
GCACTAAGAGGTTCGATAATCCTGTACTGCCATTTAATGATGGAGTTTACTTCGCGAGC
ATCGAGAAGAGCAACATTATCCGGGGGTGGATCTTTGGAACAACTTTGGACAGTAAAAC
TCAATCTCTCCTTATTGTTAATAACGCAACCAACGTAGTCATCAAGGTTTGCGAGTTCCA
GTTTTGCAATGACCCTTTTCTGGACGTTTATTATCATAAGAACAACAAGTCATGGATGGA
ATCCGGCGTGTACTCTTCCGCAAATAATTGTACGTTTGAGTATGTCAGCCAGCCTTTCC
TCATGGACCTGGAAGGCAAACAGGGGAATTTTAAAAATCTGAGGGAGTTCGTCTTCAAA
AATATAGACGGGTACTTTAAAATCTACTCCAAGCACACGCCCATCAATTTAGTGAGGGA
CCTGCCCCAAGGCTTTTCTGCTCTTGAACCACTTGTGGACCTTCCCATCGGCATCAATA
TCACTCGCTTCCAGACACTCTTGGCACTTCATAGATCTTATCTGACCCCAGGGGACAGC
TCCTCAGGTTGGACCGCCGGCGCTGCAGCTTACTACGTTGGATATTTGCAGCCACGCA
CGTTTCTTCTGAAATACAATGAGAATGGCACCATTACCGACGCCGTAGATTGCGCTCTC
GATCCTCTGAGCGAAACCAAATGTACATTAAAGAGCTTTACGGTAGAAAAGGGGATATA
CCAAACCTCTAACTTTCGGGTCCAGCCTACCGAATCAATCGTTCGGTTCCCAAACATTA
CCAACTTATGTCCATTTGGAGAAGTGTTCAATGCCACGCGGTTCGCAAGCGTTTATGCC
TGGAACAGAAAGCGCATCTCTAATTGCGTGGCCGATTACTCAGTCCTCTACAATTCAGC
TAGTTTCAGTACATTTAAATGTTACGGCGTGTCCCCCACTAAGTTGAATGACCTTTGTTT
TACTAACGTCTACGCTGACTCCTTCGTGATCAGAGGCGATGAGGTTCGGCAGATCGCT
CCTGGGCAGACCGGTAAGATCGCCGATTACAACTACAAACTCCCAGACGACTTCACTG
GCTGTGTGATTGCCTGGAACAGTAACAATTTAGACTCCAAAGTAGGGGGTAACTACAAC
TATCGATATAGACTTTTCCGGAAAAGTAACTTGAAACCTTTTGAAAGAGATATATCCACT
GAAATCTATCAGGCCGGAAGTAAGCCCTGTAATGGAGTCGAGGGCTTCAATTGCTACTT
CCCATTACAGAGCTATGGCTTCCAGCCTACGAACGGCGTCGGATATCAGCCTTACCGC
GTGGTGGTCTTGTCTTTCGAACTGTTACATGCACCTGCGACAGTGTGCGGGCCTAAGA
AATCCACCAACCTGGTGAAGAACAAATGCGTGAACTTTAATTTCAACGGACTGACTGGC
ACAGGCGTTCTGACTGAGAGCAACAAGAAATTTCTGCCCTTCCAGCAATTTGGCCGTGA
CATTGCCGACACCACGGACGCTGTACGCGATCCGCAAACCCTGGAGATCCTGGACATC
ACCCCATGCTCTTTTGGCGGGGTCAGCGTTATTACACCTGGGACCAACACAAGCAACC
AGGTTGCTGTCTTATACCAGGGGGTTAATTGCACCGAAGTGCCTGTCGCAATCCATGCT
GACCAACTTACCCCCACTTGGCGCGTTTATTCTACCGGCTCCAACGTATTCCAAACCAG
GGCTGGCTGTCTCATCGGAGCAGAGCATGTGAACAACTCTTACGAATGCGATATACCA
ATTGGCGCGGGAATCTGTGCCTCCTATCAGACACAAACAAACAGTCGCGGAAGCGCGA
GCAGTGTGGCATCACAGAGTATCATCGCATACACCATGAGCCTTGGCGCAGAGAACAG
CGTGGCCTACTCTAATAACAGTATTGCCATACCTACCAACTTCACCATTAGCGTGACGA
CCGAGATCCTGCCCGTGTCTATGACCAAGACATCTGTGGATTGCACAATGTATATCTGC
GGAGACAGCACTGAATGCTCCAATCTGCTTTTGCAGTACGGGTCTTTCTGTACCCAGCT
GAATCGAGCACTGACAGGAATCGCTGTGGAACAGGATAAGAACACCCAGGAGGTGTTC
GCCCAGGTTAAGCAGATATACAAGACCCCTCCTATAAAAGACTTCGGTGGCTTTAACTT
TTCTCAGATTCTCCCCGACCCTTCTAAGCCTTCTAAACGATCCTTCATCGAAGACCTTCT
CTTCAACAAAGTTACTTTGGCTGATGCCGGGTTCATCAAGCAGTATGGTGACTGTCTTG
GCGATATCGCCGCCAGAGATTTAATATGTGCCCAGAAGTTCAACGGTTTAACAGTCCTT
CCTCCTCTGCTGACAGACGAGATGATTGCTCAGTACACTAGTGCTCTGCTCGCCGGGA
CTATTACTTCAGGCTGGACCTTTGGCGCTGGCGCCGCCCTGCAGATTCCCTTTGCTAT
GCAAATGGCATACCGGTTCAACGGTATAGGTGTGACTCAGAATGTGCTGTATGAAAATC
AGAAACTCATTGCAAATCAGTTTAACAGTGCTATTGGGAAGATACAGGACTCCCTCTCTT
CCACCGCGTCCGCTTTAGGTAAGCTTCAAAACGTGGTAAATCAAAATGCCCAGGCTTTA
AATACCTTGGTTAAGCAACTGAGTTCAAATTTCGGAGCAATCTCTAGCGTGCTTAACGAT
ATCCTCTCTAGGCTGGATCCACCCGAGGCCGAAGTGCAGATCGATCGGCTGATCACTG
GTAGGCTGCAGAGTCTGCAGACATATGTCACCCAGCAGCTGATCAGAGCAGCCGAGAT
TCGAGCTTCAGCTAACTTAGCTGCCACAAAGATGAGTGAGTGCGTGCTGGGCCAGTCC
AAGAGGGTGGATTTCTGCGGAAAAGGCTATCATCTCATGTCCTTCCCCCAAAGCGCAC
CCCACGGGGTTGTCTTCCTGCACGTAACGTATGTGCCCGCTCAGGAGAAGAACTTTAC
TACTGCCCCCGCCATCTGCCACGATGGGAAAGCTCACTTCCCGAGAGAAGGAGTATTC
GTTTCCAATGGGACGCATTGGTTCGTCACCCAGCGGAACTTTTACGAACCACAGATCAT
TACCACGGATAATACATTTGTGAGCGGGAATTGCGATGTGGTGATTGGAATTGTAAATA
ACACAGTGTACGATCCACTGCAGTCAGAATTAGACTCAATTAAGGAGGAGTTGGATAAG
ATTCATAAAAATGGTTCAGGTGGATCAGGTGATATAGAAAAACTCCTCAATGAACAAGTA
AATAAGGAGATGCAAAGTTCTAACCTGTACATGAGCATGTCTTCTTGGTGTTACACCCAT
AGCCTCGATGGAGCGGGATTGTTCCTTTTTGACCACGCTGCGGAGGAGTATGAGCATG
CTAAAAAGCTGATAATATTTCTCAACGAGAATAATGTTCCAGTGCAATTGACAAGTATAT
CCGCCCCTGAGCATAAGTTTGAAGGGCTCACACAAATTTTCCAAAAGGCATACGAACAC
GAACAGCACATTAGCGAGTCTATTAACAACATTGTTGATCATGCAATCAAGTCCAAAGAT
CACGCCACGTTTAATTTCCTCCAGTGGTATGTAGCTGAGCAACATGAGGAAGAAGTGTT
GTTTAAGGATATTCTTGATAAAATTGAACTTATTGGAAATGAGAACCATGGCCTCTATCT
TGCGGACCAATACGTCAAGGGAATTGCCAAGTCCCGCAAGAGT
SARS-CoV-2 Delta-linker-ferritin RNA sequence (SEQ ID NO: 25)
AUGCCCCUCCUUCUCCUUCUUCCCCUCCUUUGGGCUGGAGCGCUGGCCUCUCAGUG
UGUAAAUCUCCGUACCCGAACACAACUCCCCCCCGCCUAUACCAACAGCUUCACACG
CGGAGUUUACUACCCUGACAAGGUAUUCAGGUCCAGUGUACUCCAUUCAACACAGGA
UCUGUUCCUCCCUUUUUUCAGUAAUGUGACCUGGUUUCACGCUAUCCACGUAAGCG
GCACGAAUGGCACUAAGAGGUUCGAUAAUCCUGUACUGCCAUUUAAUGAUGGAGUU
UACUUCGCGAGCAUCGAGAAGAGCAACAUUAUCCGGGGGUGGAUCUUUGGAACAAC
UUUGGACAGUAAAACUCAAUCUCUCCUUAUUGUUAAUAACGCAACCAACGUAGUCAU
CAAGGUUUGCGAGUUCCAGUUUUGCAAUGACCCUUUUCUGGACGUUUAUUAUCAUA
AGAACAACAAGUCAUGGAUGGAAUCCGGCGUGUACUCUUCCGCAAAUAAUUGUACGU
UUGAGUAUGUCAGCCAGCCUUUCCUCAUGGACCUGGAAGGCAAACAGGGGAAUUUU
AAAAAUCUGAGGGAGUUCGUCUUCAAAAAUAUAGACGGGUACUUUAAAAUCUACUCC
AAGCACACGCCCAUCAAUUUAGUGAGGGACCUGCCCCAAGGCUUUUCUGCUCUUGA
ACCACUUGUGGACCUUCCCAUCGGCAUCAAUAUCACUCGCUUCCAGACACUCUUGGC
ACUUCAUAGAUCUUAUCUGACCCCAGGGGACAGCUCCUCAGGUUGGACCGCCGGCG
CUGCAGCUUACUACGUUGGAUAUUUGCAGCCACGCACGUUUCUUCUGAAAUACAAUG
AGAAUGGCACCAUUACCGACGCCGUAGAUUGCGCUCUCGAUCCUCUGAGCGAAACC
AAAUGUACAUUAAAGAGCUUUACGGUAGAAAAGGGGAUAUACCAAACCUCUAACUUU
CGGGUCCAGCCUACCGAAUCAAUCGUUCGGUUCCCAAACAUUACCAACUUAUGUCCA
UUUGGAGAAGUGUUCAAUGCCACGCGGUUCGCAAGCGUUUAUGCCUGGAACAGAAA
GCGCAUCUCUAAUUGCGUGGCCGAUUACUCAGUCCUCUACAAUUCAGCUAGUUUCA
GUACAUUUAAAUGUUACGGCGUGUCCCCCACUAAGUUGAAUGACCUUUGUUUUACUA
ACGUCUACGCUGACUCCUUCGUGAUCAGAGGCGAUGAGGUUCGGCAGAUCGCUCCU
GGGCAGACCGGUAAGAUCGCCGAUUACAACUACAAACUCCCAGACGACUUCACUGGC
UGUGUGAUUGCCUGGAACAGUAACAAUUUAGACUCCAAAGUAGGGGGUAACUACAAC
UAUCGAUAUAGACUUUUCCGGAAAAGUAACUUGAAACCUUUUGAAAGAGAUAUAUCC
ACUGAAAUCUAUCAGGCCGGAAGUAAGCCCUGUAAUGGAGUCGAGGGCUUCAAUUG
CUACUUCCCAUUACAGAGCUAUGGCUUCCAGCCUACGAACGGCGUCGGAUAUCAGC
CUUACCGCGUGGUGGUCUUGUCUUUCGAACUGUUACAUGCACCUGCGACAGUGUGC
GGGCCUAAGAAAUCCACCAACCUGGUGAAGAACAAAUGCGUGAACUUUAAUUUCAAC
GGACUGACUGGCACAGGCGUUCUGACUGAGAGCAACAAGAAAUUUCUGCCCUUCCA
GCAAUUUGGCCGUGACAUUGCCGACACCACGGACGCUGUACGCGAUCCGCAAACCC
UGGAGAUCCUGGACAUCACCCCAUGCUCUUUUGGCGGGGUCAGCGUUAUUACACCU
GGGACCAACACAAGCAACCAGGUUGCUGUCUUAUACCAGGGGGUUAAUUGCACCGA
AGUGCCUGUCGCAAUCCAUGCUGACCAACUUACCCCCACUUGGCGCGUUUAUUCUA
CCGGCUCCAACGUAUUCCAAACCAGGGCUGGCUGUCUCAUCGGAGCAGAGCAUGUG
AACAACUCUUACGAAUGCGAUAUACCAAUUGGCGCGGGAAUCUGUGCCUCCUAUCAG
ACACAAACAAACAGUCGCGGAAGCGCGAGCAGUGUGGCAUCACAGAGUAUCAUCGCA
UACACCAUGAGCCUUGGCGCAGAGAACAGCGUGGCCUACUCUAAUAACAGUAUUGCC
AUACCUACCAACUUCACCAUUAGCGUGACGACCGAGAUCCUGCCCGUGUCUAUGACC
AAGACAUCUGUGGAUUGCACAAUGUAUAUCUGCGGAGACAGCACUGAAUGCUCCAAU
CUGCUUUUGCAGUACGGGUCUUUCUGUACCCAGCUGAAUCGAGCACUGACAGGAAU
CGCUGUGGAACAGGAUAAGAACACCCAGGAGGUGUUCGCCCAGGUUAAGCAGAUAU
ACAAGACCCCUCCUAUAAAAGACUUCGGUGGCUUUAACUUUUCUCAGAUUCUCCCCG
ACCCUUCUAAGCCUUCUAAACGAUCCUUCAUCGAAGACCUUCUCUUCAACAAAGUUA
CUUUGGCUGAUGCCGGGUUCAUCAAGCAGUAUGGUGACUGUCUUGGCGAUAUCGCC
GCCAGAGAUUUAAUAUGUGCCCAGAAGUUCAACGGUUUAACAGUCCUUCCUCCUCUG
CUGACAGACGAGAUGAUUGCUCAGUACACUAGUGCUCUGCUCGCCGGGACUAUUAC
UUCAGGCUGGACCUUUGGCGCUGGCGCCGCCCUGCAGAUUCCCUUUGCUAUGCAAA
UGGCAUACCGGUUCAACGGUAUAGGUGUGACUCAGAAUGUGCUGUAUGAAAAUCAG
AAACUCAUUGCAAAUCAGUUUAACAGUGCUAUUGGGAAGAUACAGGACUCCCUCUCU
UCCACCGCGUCCGCUUUAGGUAAGCUUCAAAACGUGGUAAAUCAAAAUGCCCAGGCU
UUAAAUACCUUGGUUAAGCAACUGAGUUCAAAUUUCGGAGCAAUCUCUAGCGUGCUU
AACGAUAUCCUCUCUAGGCUGGAUCCACCCGAGGCCGAAGUGCAGAUCGAUCGGCU
GAUCACUGGUAGGCUGCAGAGUCUGCAGACAUAUGUCACCCAGCAGCUGAUCAGAG
CAGCCGAGAUUCGAGCUUCAGCUAACUUAGCUGCCACAAAGAUGAGUGAGUGCGUG
CUGGGCCAGUCCAAGAGGGUGGAUUUCUGCGGAAAAGGCUAUCAUCUCAUGUCCUU
CCCCCAAAGCGCACCCCACGGGGUUGUCUUCCUGCACGUAACGUAUGUGCCCGCUC
AGGAGAAGAACUUUACUACUGCCCCCGCCAUCUGCCACGAUGGGAAAGCUCACUUCC
CGAGAGAAGGAGUAUUCGUUUCCAAUGGGACGCAUUGGUUCGUCACCCAGCGGAAC
UUUUACGAACCACAGAUCAUUACCACGGAUAAUACAUUUGUGAGCGGGAAUUGCGAU
GUGGUGAUUGGAAUUGUAAAUAACACAGUGUACGAUCCACUGCAGUCAGAAUUAGAC
UCAAUUAAGGAGGAGUUGGAUAAGAUUCAUAAAAAUGGUUCAGGUGGAUCAGGUGAU
AUAGAAAAACUCCUCAAUGAACAAGUAAAUAAGGAGAUGCAAAGUUCUAACCUGUACA
UGAGCAUGUCUUCUUGGUGUUACACCCAUAGCCUCGAUGGAGCGGGAUUGUUCCUU
UUUGACCACGCUGCGGAGGAGUAUGAGCAUGCUAAAAAGCUGAUAAUAUUUCUCAAC
GAGAAUAAUGUUCCAGUGCAAUUGACAAGUAUAUCCGCCCCUGAGCAUAAGUUUGAA
GGGCUCACACAAAUUUUCCAAAAGGCAUACGAACACGAACAGCACAUUAGCGAGUCU
AUUAACAACAUUGUUGAUCAUGCAAUCAAGUCCAAAGAUCACGCCACGUUUAAUUUC
CUCCAGUGGUAUGUAGCUGAGCAACAUGAGGAAGAAGUGUUGUUUAAGGAUAUUCU
UGAUAAAAUUGAACUUAUUGGAAAUGAGAACCAUGGCCUCUAUCUUGCGGACCAAUA
CGUCAAGGGAAUUGCCAAGUCCCGCAAGAGU
SARS-CoV-2 Omicron BA.4/5-linker-ferritin DNA sequence (SEQ ID NO: 26)
ATGCCCCTCCTTCTCCTTCTTCCCCTCCTTTGGGCTGGAGCGCTGGCCTCTCAGTGTGT
AAATCTCATTACCCGCACGCAGCTACCACCTGCCTATACAAACTCCTTCACCCGCGGTG
TCTATTACCCCGACAAGGTTTTTCGATCCTCAGTTCTTCATTCCACCCAGGATCTTTTCC
TTCCGTTCTTCTCCAACGTCACCTGGTTTCATGCTATTCATGTGTCAGGGACCAACGGA
ACTAAAAGGTTCGACAATCCCGTACTGCCTTTCAACGACGGGGTGTATTTCGCATCAAC
AGAAAAAAGCAACATTATCAGAGGCTGGATTTTTGGCACTACACTCGACTCAAAGACCC
AAAGTTTGCTTATTGTTAATAACGCAACGAATGTCGTAATTAAAGTCTGTGAATTCCAGT
TTTGTAACGACCCATTTCTGGATGTATACTATCATAAGAACAATAAGTCCTGGATGGAGT
CAGAGTTCAGAGTCTACTCATCAGCAAACAATTGCACTTTTGAGTACGTATCTCAGCCAT
TTCTGATGGACCTGGAAGGCAAGCAAGGGAACTTCAAAAATCTCCGAGAGTTTGTTTTC
AAAAACATAGATGGTTACTTCAAGATCTATTCTAAACACACCCCGATTAATCTTGGCCGG
GACCTGCCCCAGGGTTTCTCTGCCCTCGAGCCCCTTGTAGACTTGCCCATTGGGATAA
ATATTACTAGGTTTCAGACACTGCTTGCGCTGCACAGGTCATACCTCACTCCTGGGGAT
TCATCAAGCGGCTGGACTGCAGGTGCCGCTGCATACTACGTGGGATACCTCCAACCCC
GAACTTTTTTGCTCAAGTACAATGAGAATGGGACGATAACTGATGCCGTAGACTGCGCA
CTCGATCCTCTGTCTGAGACGAAGTGCACACTGAAGAGTTTCACGGTGGAAAAGGGTA
TATATCAAACATCCAATTTCCGAGTCCAGCCCACTGAAAGTATTGTTAGGTTCCCGAACA
TCACAAATCTCTGTCCATTTGACGAGGTATTTAACGCGACACGCTTTGCTTCCGTGTAT
GCGTGGAATAGAAAGCGCATTAGCAACTGTGTCGCGGATTATTCAGTCTTGTACAACTT
TGCGCCTTTTTTCGCATTTAAATGCTACGGGGTCAGCCCCACAAAGCTGAATGATCTCT
GCTTCACAAATGTATATGCAGATAGCTTTGTCATCCGAGGTAACGAAGTTTCTCAGATTG
CTCCCGGCCAAACGGGAAACATAGCTGACTATAACTATAAGCTCCCAGACGATTTTACG
GGTTGTGTCATTGCATGGAACTCCAATAAATTGGATTCAAAGGTAGGTGGTAATTATAAC
TACAGGTATAGGCTGTTCAGGAAGTCAAACCTGAAACCGTTTGAACGAGATATCTCAAC
TGAGATTTATCAAGCGGGCAATAAACCCTGCAACGGAGTCGCGGGTGTTAATTGCTACT
TCCCATTGCAGAGTTACGGCTTTCGGCCGACTTATGGCGTCGGACATCAACCTTACAG
GGTAGTTGTTCTGTCTTTCGAACTCCTGCATGCTCCGGCAACGGTGTGTGGTCCCAAG
AAAAGTACTAACCTTGTCAAAAATAAGTGTGTGAACTTCAATTTTAACGGTCTTACAGGT
ACTGGAGTATTGACTGAGTCAAATAAGAAGTTTCTTCCATTCCAACAATTCGGCCGCGA
TATAGCTGATACAACGGATGCCGTCCGAGACCCTCAGACTCTGGAGATCCTGGACATA
ACTCCATGTTCTTTTGGAGGCGTTAGTGTGATTACCCCAGGCACTAATACCTCAAACCA
AGTAGCCGTCCTGTATCAAGGAGTCAATTGTACTGAGGTCCCTGTGGCCATCCATGCG
GACCAACTGACACCCACATGGCGCGTATATAGCACAGGATCAAACGTATTCCAGACTC
GCGCGGGTTGTCTGATCGGGGCGGAATACGTAAACAATAGCTATGAGTGTGATATACC
TATTGGAGCTGGGATTTGCGCAAGCTATCAAACACAAACGAAATCCCACGGATCGGCAT
CATCAGTTGCATCTCAATCTATTATCGCTTATACAATGTCTCTTGGCGCTGAGAACTCCG
TTGCTTACAGCAATAATAGCATTGCCATACCGACGAACTTTACTATTAGCGTAACGACTG
AGATACTCCCGGTGTCTATGACAAAAACTAGCGTTGATTGTACAATGTACATTTGTGGG
GATAGTACAGAGTGCTCAAACTTGCTTCTCCAGTACGGGTCATTCTGTACCCAACTTAA
ACGCGCATTGACTGGTATTGCCGTCGAGCAAGACAAAAACACACAAGAGGTTTTCGCT
CAAGTAAAACAAATTTACAAAACGCCCCCTATAAAATATTTTGGCGGTTTCAACTTTTCT
CAAATTCTGCCCGATCCTTCTAAGCCTAGTAAGCGAAGCTTCATTGAAGACCTGCTTTT
CAACAAAGTAACCTTGGCTGACGCGGGTTTCATAAAACAGTATGGAGACTGTCTCGGG
GACATAGCCGCAAGGGACTTGATTTGTGCCCAAAAATTCAACGGACTGACAGTTCTTCC
TCCACTTTTGACTGATGAGATGATAGCACAATACACTTCTGCTTTGCTGGCCGGTACAA
TAACGAGTGGGTGGACCTTCGGGGCTGGGGCTGCCCTCCAAATACCCTTCGCTATGCA
AATGGCATATCGATTTAATGGCATCGGGGTTACACAGAACGTCCTGTATGAAAACCAAA
AGCTCATTGCCAACCAGTTTAACTCAGCAATCGGAAAAATTCAGGACAGCCTGAGTAGT
ACCGCCAGCGCCTTGGGAAAGTTGCAGGATGTCGTCAACCATAACGCCCAAGCATTGA
ATACGTTGGTCAAACAGCTCTCCTCTAAGTTCGGTGCTATATCCTCAGTCCTCAACGAT
ATCCTGAGCAGGCTCGATCCCCCTGAGGCCGAAGTGCAGATCGATCGGCTGATCACTG
GTAGGCTGCAGAGTCTGCAGACATATGTCACCCAGCAGCTGATCAGAGCAGCCGAGAT
TCGAGCTTCAGCTAACTTAGCTGCCACAAAGATGAGTGAGTGCGTGCTGGGCCAGTCC
AAGAGGGTGGATTTCTGCGGAAAAGGCTATCATCTCATGTCCTTCCCCCAAAGCGCAC
CCCACGGGGTTGTCTTCCTGCACGTAACGTATGTGCCCGCTCAGGAGAAGAACTTTAC
TACTGCCCCCGCCATCTGCCACGATGGGAAAGCTCACTTCCCGAGAGAAGGAGTATTC
GTTTCCAATGGGACGCATTGGTTCGTCACCCAGCGGAACTTTTACGAACCACAGATCAT
TACCACGGATAATACATTTGTGAGCGGGAATTGCGATGTGGTGATTGGAATTGTAAATA
ACACAGTGTACGATCCACTGCAGTCAGAATTAGACTCAATTAAGGAGGAGTTGGATAAG
ATTCATAAAAATGGTTCAGGTGGATCAGGTGATATAGAAAAACTCCTCAATGAACAAGTA
AATAAGGAGATGCAAAGTTCTAACCTGTACATGAGCATGTCTTCTTGGTGTTACACCCAT
AGCCTCGATGGAGCGGGATTGTTCCTTTTTGACCACGCTGCGGAGGAGTATGAGCATG
CTAAAAAGCTGATAATATTTCTCAACGAGAATAATGTTCCAGTGCAATTGACAAGTATAT
CCGCCCCTGAGCATAAGTTTGAAGGGCTCACACAAATTTTCCAAAAGGCATACGAACAC
GAACAGCACATTAGCGAGTCTATTAACAACATTGTTGATCATGCAATCAAGTCCAAAGAT
CACGCCACGTTTAATTTCCTCCAGTGGTATGTAGCTGAGCAACATGAGGAAGAAGTGTT
GTTTAAGGATATTCTTGATAAAATTGAACTTATTGGAAATGAGAACCATGGCCTCTATCT
TGCGGACCAATACGTCAAGGGAATTGCCAAGTCCCGCAAGAGT
SARS-CoV-2 Omicron BA.4/5-linker-ferritin RNA sequence (SEQ ID NO: 27)
AUGCCCCUCCUUCUCCUUCUUCCCCUCCUUUGGGCUGGAGCGCUGGCCUCUCAGUG
UGUAAAUCUCAUUACCCGCACGCAGCUACCACCUGCCUAUACAAACUCCUUCACCCG
CGGUGUCUAUUACCCCGACAAGGUUUUUCGAUCCUCAGUUCUUCAUUCCACCCAGG
AUCUUUUCCUUCCGUUCUUCUCCAACGUCACCUGGUUUCAUGCUAUUCAUGUGUCA
GGGACCAACGGAACUAAAAGGUUCGACAAUCCCGUACUGCCUUUCAACGACGGGGU
GUAUUUCGCAUCAACAGAAAAAAGCAACAUUAUCAGAGGCUGGAUUUUUGGCACUAC
ACUCGACUCAAAGACCCAAAGUUUGCUUAUUGUUAAUAACGCAACGAAUGUCGUAAU
UAAAGUCUGUGAAUUCCAGUUUUGUAACGACCCAUUUCUGGAUGUAUACUAUCAUAA
GAACAAUAAGUCCUGGAUGGAGUCAGAGUUCAGAGUCUACUCAUCAGCAAACAAUUG
CACUUUUGAGUACGUAUCUCAGCCAUUUCUGAUGGACCUGGAAGGCAAGCAAGGGA
ACUUCAAAAAUCUCCGAGAGUUUGUUUUCAAAAACAUAGAUGGUUACUUCAAGAUCU
AUUCUAAACACACCCCGAUUAAUCUUGGCCGGGACCUGCCCCAGGGUUUCUCUGCC
CUCGAGCCCCUUGUAGACUUGCCCAUUGGGAUAAAUAUUACUAGGUUUCAGACACU
GCUUGCGCUGCACAGGUCAUACCUCACUCCUGGGGAUUCAUCAAGCGGCUGGACUG
CAGGUGCCGCUGCAUACUACGUGGGAUACCUCCAACCCCGAACUUUUUUGCUCAAG
UACAAUGAGAAUGGGACGAUAACUGAUGCCGUAGACUGCGCACUCGAUCCUCUGUC
UGAGACGAAGUGCACACUGAAGAGUUUCACGGUGGAAAAGGGUAUAUAUCAAACAUC
CAAUUUCCGAGUCCAGCCCACUGAAAGUAUUGUUAGGUUCCCGAACAUCACAAAUCU
CUGUCCAUUUGACGAGGUAUUUAACGCGACACGCUUUGCUUCCGUGUAUGCGUGGA
AUAGAAAGCGCAUUAGCAACUGUGUCGCGGAUUAUUCAGUCUUGUACAACUUUGCG
CCUUUUUUCGCAUUUAAAUGCUACGGGGUCAGCCCCACAAAGCUGAAUGAUCUCUG
CUUCACAAAUGUAUAUGCAGAUAGCUUUGUCAUCCGAGGUAACGAAGUUUCUCAGAU
UGCUCCCGGCCAAACGGGAAACAUAGCUGACUAUAACUAUAAGCUCCCAGACGAUUU
UACGGGUUGUGUCAUUGCAUGGAACUCCAAUAAAUUGGAUUCAAAGGUAGGUGGUA
AUUAUAACUACAGGUAUAGGCUGUUCAGGAAGUCAAACCUGAAACCGUUUGAACGAG
AUAUCUCAACUGAGAUUUAUCAAGCGGGCAAUAAACCCUGCAACGGAGUCGCGGGU
GUUAAUUGCUACUUCCCAUUGCAGAGUUACGGCUUUCGGCCGACUUAUGGCGUCGG
ACAUCAACCUUACAGGGUAGUUGUUCUGUCUUUCGAACUCCUGCAUGCUCCGGCAA
CGGUGUGUGGUCCCAAGAAAAGUACUAACCUUGUCAAAAAUAAGUGUGUGAACUUCA
AUUUUAACGGUCUUACAGGUACUGGAGUAUUGACUGAGUCAAAUAAGAAGUUUCUUC
CAUUCCAACAAUUCGGCCGCGAUAUAGCUGAUACAACGGAUGCCGUCCGAGACCCUC
AGACUCUGGAGAUCCUGGACAUAACUCCAUGUUCUUUUGGAGGCGUUAGUGUGAUU
ACCCCAGGCACUAAUACCUCAAACCAAGUAGCCGUCCUGUAUCAAGGAGUCAAUUGU
ACUGAGGUCCCUGUGGCCAUCCAUGCGGACCAACUGACACCCACAUGGCGCGUAUA
UAGCACAGGAUCAAACGUAUUCCAGACUCGCGCGGGUUGUCUGAUCGGGGCGGAAU
ACGUAAACAAUAGCUAUGAGUGUGAUAUACCUAUUGGAGCUGGGAUUUGCGCAAGC
UAUCAAACACAAACGAAAUCCCACGGAUCGGCAUCAUCAGUUGCAUCUCAAUCUAUU
AUCGCUUAUACAAUGUCUCUUGGCGCUGAGAACUCCGUUGCUUACAGCAAUAAUAGC
AUUGCCAUACCGACGAACUUUACUAUUAGCGUAACGACUGAGAUACUCCCGGUGUCU
AUGACAAAAACUAGCGUUGAUUGUACAAUGUACAUUUGUGGGGAUAGUACAGAGUGC
UCAAACUUGCUUCUCCAGUACGGGUCAUUCUGUACCCAACUUAAACGCGCAUUGACU
GGUAUUGCCGUCGAGCAAGACAAAAACACACAAGAGGUUUUCGCUCAAGUAAAACAA
AUUUACAAAACGCCCCCUAUAAAAUAUUUUGGCGGUUUCAACUUUUCUCAAAUUCUG
CCCGAUCCUUCUAAGCCUAGUAAGCGAAGCUUCAUUGAAGACCUGCUUUUCAACAAA
GUAACCUUGGCUGACGCGGGUUUCAUAAAACAGUAUGGAGACUGUCUCGGGGACAU
AGCCGCAAGGGACUUGAUUUGUGCCCAAAAAUUCAACGGACUGACAGUUCUUCCUCC
ACUUUUGACUGAUGAGAUGAUAGCACAAUACACUUCUGCUUUGCUGGCCGGUACAAU
AACGAGUGGGUGGACCUUCGGGGCUGGGGCUGCCCUCCAAAUACCCUUCGCUAUGC
AAAUGGCAUAUCGAUUUAAUGGCAUCGGGGUUACACAGAACGUCCUGUAUGAAAACC
AAAAGCUCAUUGCCAACCAGUUUAACUCAGCAAUCGGAAAAAUUCAGGACAGCCUGA
GUAGUACCGCCAGCGCCUUGGGAAAGUUGCAGGAUGUCGUCAACCAUAACGCCCAA
GCAUUGAAUACGUUGGUCAAACAGCUCUCCUCUAAGUUCGGUGCUAUAUCCUCAGU
CCUCAACGAUAUCCUGAGCAGGCUCGAUCCCCCUGAGGCCGAAGUGCAGAUCGAUC
GGCUGAUCACUGGUAGGCUGCAGAGUCUGCAGACAUAUGUCACCCAGCAGCUGAUC
AGAGCAGCCGAGAUUCGAGCUUCAGCUAACUUAGCUGCCACAAAGAUGAGUGAGUG
CGUGCUGGGCCAGUCCAAGAGGGUGGAUUUCUGCGGAAAAGGCUAUCAUCUCAUGU
CCUUCCCCCAAAGCGCACCCCACGGGGUUGUCUUCCUGCACGUAACGUAUGUGCCC
GCUCAGGAGAAGAACUUUACUACUGCCCCCGCCAUCUGCCACGAUGGGAAAGCUCA
CUUCCCGAGAGAAGGAGUAUUCGUUUCCAAUGGGACGCAUUGGUUCGUCACCCAGC
GGAACUUUUACGAACCACAGAUCAUUACCACGGAUAAUACAUUUGUGAGCGGGAAUU
GCGAUGUGGUGAUUGGAAUUGUAAAUAACACAGUGUACGAUCCACUGCAGUCAGAAU
UAGACUCAAUUAAGGAGGAGUUGGAUAAGAUUCAUAAAAAUGGUUCAGGUGGAUCAG
GUGAUAUAGAAAAACUCCUCAAUGAACAAGUAAAUAAGGAGAUGCAAAGUUCUAACCU
GUACAUGAGCAUGUCUUCUUGGUGUUACACCCAUAGCCUCGAUGGAGCGGGAUUGU
UCCUUUUUGACCACGCUGCGGAGGAGUAUGAGCAUGCUAAAAAGCUGAUAAUAUUUC
UCAACGAGAAUAAUGUUCCAGUGCAAUUGACAAGUAUAUCCGCCCCUGAGCAUAAGU
UUGAAGGGCUCACACAAAUUUUCCAAAAGGCAUACGAACACGAACAGCACAUUAGCG
AGUCUAUUAACAACAUUGUUGAUCAUGCAAUCAAGUCCAAAGAUCACGCCACGUUUA
AUUUCCUCCAGUGGUAUGUAGCUGAGCAACAUGAGGAAGAAGUGUUGUUUAAGGAU
AUUCUUGAUAAAAUUGAACUUAUUGGAAAUGAGAACCAUGGCCUCUAUCUUGCGGAC
CAAUACGUCAAGGGAAUUGCCAAGUCCCGCAAGAGU
Linker amino acid sequence: GSGGSG (SEQ ID NO: 28).
Linker DNA sequence: GGTTCAGGTGGATCAGGT (SEQ ID NO: 29)
Linker RNA sequence: GGUUCAGGUGGAUCAGGU (SEQ ID NO: 30)
Ferritin subunit, DNA sequence (SEQ ID NO: 31):
GATATAGAAAAACTCCTCAATGAACAAGTAAATAAGGAGATGCAAAGTTCTAACCTGTAC
ATGAGCATGTCTTCTTGGTGTTACACCCATAGCCTCGATGGAGCGGGATTGTTCCTTTT
TGACCACGCTGCGGAGGAGTATGAGCATGCTAAAAAGCTGATAATATTTCTCAACGAGA
ATAATGTTCCAGTGCAATTGACAAGTATATCCGCCCCTGAGCATAAGTTTGAAGGGCTC
ACACAAATTTTCCAAAAGGCATACGAACACGAACAGCACATTAGCGAGTCTATTAACAA
CATTGTTGATCATGCAATCAAGTCCAAAGATCACGCCACGTTTAATTTCCTCCAGTGGTA
TGTAGCTGAGCAACATGAGGAAGAAGTGTTGTTTAAGGATATTCTTGATAAAATTGAACT
TATTGGAAATGAGAACCATGGCCTCTATCTTGCGGACCAATACGTCAAGGGAATTGCCA
AGTCCCGCAAGAGT
Ferritin subunit, RNA sequence (SEQ ID NO: 32):
GAUAUAGAAAAACUCCUCAAUGAACAAGUAAAUAAGGAGAUGCAAAGUUCUAACCUGU
ACAUGAGCAUGUCUUCUUGGUGUUACACCCAUAGCCUCGAUGGAGCGGGAUUGUUC
CUUUUUGACCACGCUGCGGAGGAGUAUGAGCAUGCUAAAAAGCUGAUAAUAUUUCUC
AACGAGAAUAAUGUUCCAGUGCAAUUGACAAGUAUAUCCGCCCCUGAGCAUAAGUUU
GAAGGGCUCACACAAAUUUUCCAAAAGGCAUACGAACACGAACAGCACAUUAGCGAG
UCUAUUAACAACAUUGUUGAUCAUGCAAUCAAGUCCAAAGAUCACGCCACGUUUAAU
UUCCUCCAGUGGUAUGUAGCUGAGCAACAUGAGGAAGAAGUGUUGUUUAAGGAUAU
UCUUGAUAAAAUUGAACUUAUUGGAAAUGAGAACCAUGGCCUCUAUCUUGCGGACCA
AUACGUCAAGGGAAUUGCCAAGUCCCGCAAGAGU
Ferritin subunit, protein sequence (SEQ ID NO: 33)
DIEKLLNEQVNKEMQSSNLYMSMSSWCYTHSLDGAGLFLFDHAAEEYEHAKKLIIFLNENNV
PVQLTSISAPEHKFEGLTQIFQKAYEHEQHISESINNIVDHAIKSKDHATFNFLQWYVAEQHE
EEVLFKDILDKIELIGNENHGLYLADQYVKGIAKSRKS
Leader sequence: MPLLLLLPLLWAGALA (SEQ ID NO: 34).
CHIT1 upstream ORF, longer sequence: ATGGGCTGCAGCCTGCCGCTGA (SEQ ID
NO: 35)
mRNA construct sequence encoding SARS-CoV-2 Wuhan D614G Spike-ferritin fusion
protein (SEQ ID NO: 36)
AGGAUUGUGCUGCAUCAAGCUUGCCGCCACCAUGCCCCUCCUUCUCCUUCUUCCCC
UCCUUUGGGCUGGAGCGCUGGCCUCUCAGUGUGUAAAUCUCACCACAAGAACCCAG
CUGCCCCCUGCCUAUACCAAUUCCUUCACACGGGGCGUGUACUAUCCCGACAAGGU
GUUUAGAUCUAGCGUGCUGCACUCCACACAGGAUCUGUUUCUGCCUUUUUUUCUA
ACGUGACCUGGUUCCACGCCAUCCACGUGAGCGGCACCAAUGGCACAAAGAGGUUC
GACAAUCCAGUGCUGCCCUUUAACGAUGGCGUGUACUUCGCCUCCACCGAGAAGUC
UAACAUCAUCCGCGGCUGGAUCUUUGGCACCACACUGGACAGCAAGACACAGUCCCU
GCUGAUCGUGAACAAUGCCACCAACGUGGUCAUCAAGGUGUGCGAGUUCCAGUUUU
GUAAUGAUCCAUUCCUGGGCGUGUACUAUCACAAGAACAAUAAGUCUUGGAUGGAGA
GCGAGUUUCGGGUGUAUUCCUCUGCCAACAAUUGCACAUUUGAGUACGUGUCCCAG
CCCUUCCUGAUGGACCUGGAGGGCAAGCAGGGCAAUUUCAAGAACCUGCGGGAGUU
CGUGUUUAAGAAUAUCGAUGGCUACUUCAAGAUCUACUCCAAGCACACCCCCAUCAA
CCUGGUGCGGGACCUGCCACAGGGCUUCUCUGCCCUGGAGCCACUGGUGGAUCUG
CCCAUCGGCAUCAACAUCACCCGGUUUCAGACACUGCUGGCCCUGCACAGAAGCUAC
CUGACACCAGGCGACAGCUCCUCUGGAUGGACCGCAGGAGCAGCAGCCUACUAUGU
GGGCUAUCUGCAGCCCCGGACCUUCCUGCUGAAGUACAACGAGAAUGGCACCAUCA
CAGACGCCGUGGAUUGCGCCCUGGAUCCCCUGUCUGAGACAAAGUGUACACUGAAG
AGUUUUACCGUGGAGAAGGGCAUCUAUCAGACAAGCAAUUUCAGGGUGCAGCCUAC
CGAGUCCAUCGUGCGCUUUCCCAAUAUCACAAACCUGUGCCCUUUUGGCGAGGUGU
UCAACGCCACCAGAUUCGCCAGCGUGUACGCCUGGAAUAGGAAGCGCAUCUCCAAC
UGCGUGGCCGACUAUUCUGUGCUGUACAACAGCGCCUCCUUCUCUACCUUUAAGUG
CUAUGGCGUGAGCCCCACAAAGCUGAAUGACCUGUGCUUUACCAACGUGUACGCCG
AUUCCUUCGUGAUCAGGGGCGACGAGGUGCGCCAGAUCGCACCAGGACAGACAGGC
AAGAUCGCAGACUACAAUUAUAAGCUGCCUGACGAUUUCACCGGCUGCGUGAUCGC
CUGGAACUCUAACAAUCUGGAUAGCAAAGUGGGCGGCAACUACAAUUAUCUGUACCG
GCUGUUUAGAAAGUCUAAUCUGAAGCCAUUCGAGCGGGACAUCUCCACAGAGAUCUA
CCAGGCCGGCUCUACCCCCUGCAAUGGCGUGGAGGGCUUUAACUGUUAUUUCCCUC
UGCAGAGCUACGGCUUCCAGCCAACCAACGGCGUGGGCUAUCAGCCCUACAGAGUG
GUGGUGCUGUCUUUUGAGCUGCUGCACGCACCUGCAACAGUGUGCGGACCAAAAAA
GAGCACCAAUCUGGUGAAGAACAAGUGCGUGAACUUCAACUUCAACGGCCUGACCG
GAACAGGCGUGCUGACCGAGUCCAACAAGAAGUUCCUGCCUUUUCAGCAGUUCGGC
AGGGACAUCGCAGAUACCACAGACGCCGUGCGCGACCCUCAGACCCUGGAGAUCCU
GGAUAUCACACCAUGCUCCUUCGGCGGCGUGUCUGUGAUCACACCAGGCACCAAUA
CAAGCAACCAGGUGGCCGUGCUGUAUCAGGGCGUGAAUUGUACCGAGGUGCCAGUG
GCAAUCCACGCAGAUCAGCUGACCCCUACAUGGCGGGUGUACUCUACCGGCAGCAA
CGUGUUCCAGACAAGAGCCGGAUGCCUGAUCGGAGCAGAGCACGUGAACAAUAGCU
AUGAGUGCGACAUCCCUAUCGGCGCCGGCAUCUGUGCCUCCUACCAGACCCAGACA
AACUCCCCAGGGUCUGCAUCAUCUGUGGCAAGCCAGUCCAUCAUCGCCUAUACCAU
GAGCCUGGGCGCCGAGAAUUCCGUGGCCUACUCCAACAAUUCUAUCGCCAUCCCUA
CCAACUUCACAAUCUCCGUGACCACAGAGAUCCUGCCAGUGAGCAUGACCAAGACAU
CCGUGGACUGCACAAUGUAUAUCUGUGGCGAUUCCACCGAGUGCUCUAACCUGCUG
CUGCAGUACGGCAGCUUUUGUACCCAGCUGAAUCGCGCCCUGACAGGAAUCGCAGU
GGAGCAGGAUAAGAACACACAGGAGGUGUUCGCCCAGGUGAAGCAGAUCUACAAGA
CCCCACCCAUCAAGGACUUUGGCGGCUUCAAUUUUUCCCAGAUCCUGCCCGAUCCU
AGCAAGCCAUCCAAGAGGUCUUUUAUCGAGGACCUGCUGUUCAACAAGGUGACCCU
GGCCGAUGCCGGCUUCAUCAAGCAGUAUGGCGAUUGCCUGGGCGACAUCGCAGCCC
GCGACCUGAUCUGUGCCCAGAAGUUUAAUGGCCUGACCGUGCUGCCUCCACUGCUG
ACAGAUGAGAUGAUCGCCCAGUACACAUCUGCCCUGCUGGCAGGAACCAUCACAAGC
GGAUGGACCUUCGGCGCAGGAGCCGCCCUGCAGAUCCCCUUUGCCAUGCAGAUGGC
CUAUCGGUUCAACGGCAUCGGCGUGACCCAGAAUGUGCUGUACGAGAACCAGAAGC
UGAUCGCCAAUCAGUUUAACUCCGCCAUCGGCAAGAUCCAGGACUCUCUGAGCAGCA
CAGCAAGCGCCCUGGGCAAGCUGCAGGAUGUGGUGAAUCAGAACGCCCAGGCCCUG
AAUACCCUGGUGAAGCAGCUGUCUAGCAACUUCGGCGCCAUCUCCUCUGUGCUGAA
UGAUAUCCUGAGCAGGCUGGACCCUCCAGAGGCCGAAGUGCAGAUCGAUCGGCUGA
UCACUGGUAGGCUGCAGAGUCUGCAGACAUAUGUCACCCAGCAGCUGAUCAGAGCA
GCCGAGAUUCGAGCUUCAGCUAACUUAGCUGCCACAAAGAUGAGUGAGUGCGUGCU
GGGCCAGUCCAAGAGGGUGGAUUUCUGCGGAAAAGGCUAUCAUCUCAUGUCCUUCC
CCCAAAGCGCACCCCACGGGGUUGUCUUCCUGCACGUAACGUAUGUGCCCGCUCAG
GAGAAGAACUUUACUACUGCCCCCGCCAUCUGCCACGAUGGGAAAGCUCACUUCCC
GAGAGAAGGAGUAUUCGUUUCCAAUGGGACGCAUUGGUUCGUCACCCAGCGGAACU
UUUACGAACCACAGAUCAUUACCACGGAUAAUACAUUUGUGAGCGGGAAUUGCGAUG
UGGUGAUUGGAAUUGUAAAUAACACAGUGUACGAUCCACUGCAGUCAGAAUUAGACU
CAAUUAAGGAGGAGUUGGAUAAGAUUCAUAAAAAUGGUUCAGGUGGAUCAGGUGAUA
UAGAAAAACUCCUCAAUGAACAAGUAAAUAAGGAGAUGCAAAGUUCUAACCUGUACAU
GAGCAUGUCUUCUUGGUGUUACACCCAUAGCCUCGAUGGAGCGGGAUUGUUCCUUU
UUGACCACGCUGCGGAGGAGUAUGAGCAUGCUAAAAAGCUGAUAAUAUUUCUCAACG
AGAAUAAUGUUCCAGUGCAAUUGACAAGUAUAUCCGCCCCUGAGCAUAAGUUUGAAG
GGCUCACACAAAUUUUCCAAAAGGCAUACGAACACGAACAGCACAUUAGCGAGUCUA
UUAACAACAUUGUUGAUCAUGCAAUCAAGUCCAAAGAUCACGCCACGUUUAAUUUCC
UCCAGUGGUAUGUAGCUGAGCAACAUGAGGAAGAAGUGUUGUUUAAGGAUAUUCUU
GAUAAAAUUGAACUUAUUGGAAAUGAGAACCAUGGCCUCUAUCUUGCGGACCAAUAC
GUCAAGGGAAUUGCCAAGUCCCGCAAGAGUUGAUGAUAAUAGGACUAGUGGAUCCAA
CUGGAGACUGGGUGAAAGUGACUACCAGAAAGUGAGGAAGCCUAAAUAAACCUAGCG
UACGUAAAAAAUGGAAAGAACCUAGCGUACGAAAAAAAAAAAAAAAAAAAAAAAAAAAA
AAAAAAAAAAAAAAAAAAAAAAAAAAAAAAAAAAAAAAAAAAAAAAAAA
mRNA construct sequence encoding SARS-CoV-2 Delta Spike-ferritin fusion protein
(SEQ ID NO: 37)
AGGAUUGUGCUGCAUCAAGCUUGCCGCCACCAUGCCCCUCCUUCUCCUUCUUCCCC
UCCUUUGGGCUGGAGCGCUGGCCUCUCAGUGUGUAAAUCUCCGUACCCGAACACAA
CUCCCCCCCGCCUAUACCAACAGCUUCACACGCGGAGUUUACUACCCUGACAAGGUA
UUCAGGUCCAGUGUACUCCAUUCAACACAGGAUCUGUUCCUCCCUUUUUUCAGUAAU
GUGACCUGGUUUCACGCUAUCCACGUAAGCGGCACGAAUGGCACUAAGAGGUUCGA
UAAUCCUGUACUGCCAUUUAAUGAUGGAGUUUACUUCGCGAGCAUCGAGAAAAGCAA
CAUUAUCCGGGGGUGGAUCUUUGGAACAACUUUGGACAGUAAAACUCAAUCUCUCCU
UAUUGUUAAUAACGCAACCAACGUAGUCAUCAAGGUUUGCGAGUUCCAGUUUUGCAA
UGACCCUUUUCUGGACGUUUAUUAUCAUAAGAACAACAAGUCAUGGAUGGAAUCCGG
CGUGUACUCUUCCGCAAAUAAUUGUACGUUUGAGUAUGUCAGCCAGCCUUUCCUCA
UGGACCUGGAAGGCAAACAGGGGAAUUUUAAAAAUCUGAGGGAGUUCGUCUUCAAAA
AUAUAGACGGGUACUUUAAAAUCUACUCCAAGCACACGCCCAUCAAUUUAGUGAGGG
ACCUGCCCCAAGGCUUUUCUGCUCUUGAACCACUUGUGGACCUUCCCAUCGGCAUC
AAUAUCACUCGCUUCCAGACACUCUUGGCACUUCAUAGAUCUUAUCUGACCCCAGGG
GACAGCUCCUCAGGUUGGACCGCCGGCGCUGCAGCUUACUACGUUGGAUAUUUGCA
GCCACGCACGUUUCUUCUGAAAUACAAUGAGAAUGGCACCAUUACCGACGCCGUAGA
UUGCGCUCUCGAUCCUCUGAGCGAAACCAAAUGUACAUUAAAGAGCUUUACGGUAGA
AAAGGGGAUAUACCAAACCUCUAACUUUCGGGUCCAGCCUACCGAAUCAAUCGUUCG
GUUCCCAAACAUUACCAACUUAUGUCCAUUUGGAGAAGUGUUCAAUGCCACGCGGUU
CGCAAGCGUUUAUGCCUGGAACAGAAAGCGCAUCUCUAAUUGCGUGGCCGAUUACU
CAGUCCUCUACAAUUCAGCUAGUUUCAGUACAUUUAAAUGUUACGGCGUGUCCCCCA
CUAAGUUGAAUGACCUUUGUUUUACUAACGUCUACGCUGACUCCUUCGUGAUCAGA
GGCGAUGAGGUUCGGCAGAUCGCUCCUGGGCAGACCGGUAAGAUCGCCGAUUACAA
CUACAAACUCCCAGACGACUUCACUGGCUGUGUGAUUGCCUGGAACAGUAACAAUUU
AGACUCCAAAGUAGGGGGUAACUACAACUAUCGAUAUAGACUUUUCCGGAAAAGUAA
CUUGAAACCUUUUGAAAGAGAUAUAUCCACUGAAAUCUAUCAGGCCGGAAGUAAGCC
CUGUAAUGGAGUCGAGGGCUUCAAUUGCUACUUCCCAUUACAGAGCUAUGGCUUCC
AGCCUACGAACGGCGUCGGAUAUCAGCCUUACCGCGUGGUGGUCUUGUCUUUCGAA
CUGUUACAUGCACCUGCGACAGUGUGCGGGCCUAAGAAAUCCACCAACCUGGUGAA
GAACAAAUGCGUGAACUUUAAUUUCAACGGACUGACUGGCACAGGCGUUCUGACUGA
GAGCAACAAGAAAUUUCUGCCCUUCCAGCAAUUUGGCCGUGACAUUGCCGACACCAC
GGACGCUGUACGCGAUCCGCAAACCCUGGAGAUCCUGGACAUCACCCCAUGCUCUU
UUGGCGGGGUCAGCGUUAUUACACCUGGGACCAACACAAGCAACCAGGUUGCUGUC
UUAUACCAGGGGGUUAAUUGCACCGAAGUGCCUGUCGCAAUCCAUGCUGACCAACU
UACCCCCACUUGGCGCGUUUAUUCUACCGGCUCCAACGUAUUCCAAACCAGGGCUG
GCUGUCUCAUCGGAGCAGAGCAUGUGAACAACUCUUACGAAUGCGAUAUACCAAUUG
GCGCGGGAAUCUGUGCCUCCUAUCAGACACAAACAAACAGUCGCGGAAGCGCGAGC
AGUGUGGCAUCACAGAGUAUCAUCGCAUACACCAUGAGCCUUGGCGCAGAGAACAG
CGUGGCCUACUCUAAUAACAGUAUUGCCAUACCUACCAACUUCACCAUUAGCGUGAC
GACCGAGAUCCUGCCCGUGUCUAUGACCAAGACAUCUGUGGAUUGCACAAUGUAUA
UCUGCGGAGACAGCACUGAAUGCUCCAAUCUGCUUUUGCAGUACGGGUCUUUCUGU
ACCCAGCUGAAUCGAGCACUGACAGGAAUCGCUGUGGAACAGGAUAAGAACACCCAG
GAGGUGUUCGCCCAGGUUAAGCAGAUAUACAAGACCCCUCCUAUAAAAGACUUCGGU
GGCUUUAACUUUUCUCAGAUUCUCCCCGACCCUUCUAAGCCUUCUAAACGAUCCUUC
AUCGAAGACCUUCUCUUCAACAAAGUUACUUUGGCUGAUGCCGGGUUCAUCAAGCAG
UAUGGUGACUGUCUUGGCGAUAUCGCCGCCAGAGAUUUAAUAUGUGCCCAGAAGUU
CAACGGUUUAACAGUCCUUCCUCCUCUGCUGACAGACGAGAUGAUUGCUCAGUACAC
UAGUGCUCUGCUCGCCGGGACUAUUACUUCAGGCUGGACCUUUGGCGCUGGCGCC
GCCCUGCAGAUUCCCUUUGCUAUGCAAAUGGCAUACCGGUUCAACGGUAUAGGUGU
GACUCAGAAUGUGCUGUAUGAAAAUCAGAAACUCAUUGCAAAUCAGUUUAACAGUGC
UAUUGGGAAGAUACAGGACUCCCUCUCUUCCACCGCGUCCGCUUUAGGUAAGCUUC
AAAACGUGGUAAAUCAAAAUGCCCAGGCUUUAAAUACCUUGGUUAAGCAACUGAGUU
CAAAUUUCGGAGCAAUCUCUAGCGUGCUUAACGAUAUCCUCUCUAGGCUGGAUCCAC
CCGAGGCCGAAGUGCAGAUCGAUCGGCUGAUCACUGGUAGGCUGCAGAGUCUGCAG
ACAUAUGUCACCCAGCAGCUGAUCAGAGCAGCCGAGAUUCGAGCUUCAGCUAACUUA
GCUGCCACAAAGAUGAGUGAGUGCGUGCUGGGCCAGUCCAAGAGGGUGGAUUUCUG
CGGAAAAGGCUAUCAUCUCAUGUCCUUCCCCCAAAGCGCACCCCACGGGGUUGUCU
UCCUGCACGUAACGUAUGUGCCCGCUCAGGAGAAGAACUUUACUACUGCCCCCGCC
AUCUGCCACGAUGGGAAAGCUCACUUCCCGAGAGAAGGAGUAUUCGUUUCCAAUGG
GACGCAUUGGUUCGUCACCCAGCGGAACUUUUACGAACCACAGAUCAUUACCACGGA
UAAUACAUUUGUGAGCGGGAAUUGCGAUGUGGUGAUUGGAAUUGUAAAUAACACAG
UGUACGAUCCACUGCAGUCAGAAUUAGACUCAAUUAAGGAGGAGUUGGAUAAGAUUC
AUAAAAAUGGUUCAGGUGGAUCAGGUGAUAUAGAAAAACUCCUCAAUGAACAAGUAA
AUAAGGAGAUGCAAAGUUCUAACCUGUACAUGAGCAUGUCUUCUUGGUGUUACACCC
AUAGCCUCGAUGGAGCGGGAUUGUUCCUUUUUGACCACGCUGCGGAGGAGUAUGAG
CAUGCUAAAAAGCUGAUAAUAUUUCUCAACGAGAAUAAUGUUCCAGUGCAAUUGACA
AGUAUAUCCGCCCCUGAGCAUAAGUUUGAAGGGCUCACACAAAUUUUCCAAAAGGCA
UACGAACACGAACAGCACAUUAGCGAGUCUAUUAACAACAUUGUUGAUCAUGCAAUC
AAGUCCAAAGAUCACGCCACGUUUAAUUUCCUCCAGUGGUAUGUAGCUGAGCAACAU
GAGGAAGAAGUGUUGUUUAAGGAUAUUCUUGAUAAAAUUGAACUUAUUGGAAAUGAG
AACCAUGGCCUCUAUCUUGCGGACCAAUACGUCAAGGGAAUUGCCAAGUCCCGCAAG
AGUUGAUGAUAAUAGGACUAGUGGAUCCAACUGGAGACUGGGUGAAAGUGACUACC
AGAAAGUGAGGAAGCCUAAAUAAACCUAGCGUACGUAAAAAAUGGAAAGAACCUAGC
GUACGAAAAAAAAAAAAAAAAAAAAAAAAAAAAAAAAAAAAAAAAAAAAAAAAAAAAAAAA
AAAAAAAAAAAAAAAAAAAAA
mRNA construct sequence encoding SARS-CoV-2 Omicron BA.4/5 Spike-ferritin fusion
protein (SEQ ID NO: 38)
AGGAUUGUGCUGCAUCAAGCUUGCCGCCACCAUGCCCCUCCUUCUCCUUCUUCCCC
UCCUUUGGGCUGGAGCGCUGGCCUCUCAGUGUGUAAAUCUCAUUACCCGCACGCAG
CUACCACCUGCCUAUACAAACUCCUUCACCCGCGGUGUCUAUUACCCCGACAAGGUU
UUUCGAUCCUCAGUUCUUCAUUCCACCCAGGAUCUUUUCCUUCCGUUCUUCUCCAAC
GUCACCUGGUUUCAUGCUAUUCAUGUGUCAGGGACCAACGGAACUAAAAGGUUCGA
CAAUCCCGUACUGCCUUUCAACGACGGGGUGUAUUUCGCAUCAACAGAAAAAAGCAA
CAUUAUCAGAGGCUGGAUUUUUGGCACUACACUCGACUCAAAGACCCAAAGUUUGCU
UAUUGUUAAUAACGCAACGAAUGUCGUAAUUAAAGUCUGUGAAUUCCAGUUUUGUAA
CGACCCAUUUCUGGAUGUAUACUAUCAUAAGAACAAUAAGUCCUGGAUGGAGUCAGA
GUUCAGAGUCUACUCAUCAGCAAACAAUUGCACUUUUGAGUACGUAUCUCAGCCAUU
UCUGAUGGACCUGGAAGGCAAGCAAGGGAACUUCAAAAAUCUCCGAGAGUUUGUUU
UCAAAAACAUAGAUGGUUACUUCAAGAUCUAUUCUAAACACACCCCGAUUAAUCUUG
GCCGGGACCUGCCCCAGGGUUUCUCUGCCCUCGAGCCCCUUGUAGACUUGCCCAUU
GGGAUAAAUAUUACUAGGUUUCAGACACUGCUUGCGCUGCACAGGUCAUACCUCACU
CCUGGGGAUUCAUCAAGCGGCUGGACUGCAGGUGCCGCUGCAUACUACGUGGGAUA
CCUCCAACCCCGAACUUUUUUGCUCAAGUACAAUGAGAAUGGGACGAUAACUGAUGC
CGUAGACUGCGCACUCGAUCCUCUGUCUGAGACGAAGUGCACACUGAAGAGUUUCA
CGGUGGAAAAGGGUAUAUAUCAAACAUCCAAUUUCCGAGUCCAGCCCACUGAAAGUA
UUGUUAGGUUCCCGAACAUCACAAAUCUCUGUCCAUUUGACGAGGUAUUUAACGCGA
CACGCUUUGCUUCCGUGUAUGCGUGGAAUAGAAAGCGCAUUAGCAACUGUGUCGCG
GAUUAUUCAGUCUUGUACAACUUUGCGCCUUUUUUCGCAUUUAAAUGCUACGGGGU
CAGCCCCACAAAGCUGAAUGAUCUCUGCUUCACAAAUGUAUAUGCAGAUAGCUUUGU
CAUCCGAGGUAACGAAGUUUCUCAGAUUGCUCCCGGCCAAACGGGAAACAUAGCUGA
CUAUAACUAUAAGCUCCCAGACGAUUUUACGGGUUGUGUCAUUGCAUGGAACUCCAA
UAAAUUGGAUUCAAAGGUAGGUGGUAAUUAUAACUACAGGUAUAGGCUGUUCAGGAA
GUCAAACCUGAAACCGUUUGAACGAGAUAUCUCAACUGAGAUUUAUCAAGCGGGCAA
UAAACCCUGCAACGGAGUCGCGGGUGUUAAUUGCUACUUCCCAUUGCAGAGUUACG
GCUUUCGGCCGACUUAUGGCGUCGGACAUCAACCUUACAGGGUAGUUGUUCUGUCU
UUCGAACUCCUGCAUGCUCCGGCAACGGUGUGUGGUCCCAAGAAAAGUACUAACCU
UGUCAAAAAUAAGUGUGUGAACUUCAAUUUUAACGGUCUUACAGGUACUGGAGUAUU
GACUGAGUCAAAUAAGAAGUUUCUUCCAUUCCAACAAUUCGGCCGCGAUAUAGCUGA
UACAACGGAUGCCGUCCGAGACCCUCAGACUCUGGAGAUCCUGGACAUAACUCCAU
GUUCUUUUGGAGGCGUUAGUGUGAUUACCCCAGGCACUAAUACCUCAAACCAAGUA
GCCGUCCUGUAUCAAGGAGUCAAUUGUACUGAGGUCCCUGUGGCCAUCCAUGCGGA
CCAACUGACACCCACAUGGCGCGUAUAUAGCACAGGAUCAAACGUAUUCCAGACUCG
CGCGGGUUGUCUGAUCGGGGCGGAAUACGUAAACAAUAGCUAUGAGUGUGAUAUAC
CUAUUGGAGCUGGGAUUUGCGCAAGCUAUCAAACACAAACGAAAUCCCACGGAUCGG
CAUCAUCAGUUGCAUCUCAAUCUAUUAUCGCUUAUACAAUGUCUCUUGGCGCUGAGA
ACUCCGUUGCUUACAGCAAUAAUAGCAUUGCCAUACCGACGAACUUUACUAUUAGCG
UAACGACUGAGAUACUCCCGGUGUCUAUGACAAAAACUAGCGUUGAUUGUACAAUGU
ACAUUUGUGGGGAUAGUACAGAGUGCUCAAACUUGCUUCUCCAGUACGGGUCAUUC
UGUACCCAACUUAAACGCGCAUUGACUGGUAUUGCCGUCGAGCAAGACAAAAACACA
CAAGAGGUUUUCGCUCAAGUAAAACAAAUUUACAAAACGCCCCCUAUAAAAUAUUUUG
GCGGUUUCAACUUUUCUCAAAUUCUGCCCGAUCCUUCUAAGCCUAGUAAGCGAAGCU
UCAUUGAAGACCUGCUUUUCAACAAAGUAACCUUGGCUGACGCGGGUUUCAUAAAAC
AGUAUGGAGACUGUCUCGGGGACAUAGCCGCAAGGGACUUGAUUUGUGCCCAAAAA
UUCAACGGACUGACAGUUCUUCCUCCACUUUUGACUGAUGAGAUGAUAGCACAAUAC
ACUUCUGCUUUGCUGGCCGGUACAAUAACGAGUGGGUGGACCUUCGGGGCUGGGG
CUGCCCUCCAAAUACCCUUCGCUAUGCAAAUGGCAUAUCGAUUUAAUGGCAUCGGG
GUUACACAGAACGUCCUGUAUGAAAACCAAAAGCUCAUUGCCAACCAGUUUAACUCA
GCAAUCGGAAAAAUUCAGGACAGCCUGAGUAGUACCGCCAGCGCCUUGGGAAAGUU
GCAGGAUGUCGUCAACCAUAACGCCCAAGCAUUGAAUACGUUGGUCAAACAGCUCUC
CUCUAAGUUCGGUGCUAUAUCCUCAGUCCUCAACGAUAUCCUGAGCAGGCUCGAUC
CCCCUGAGGCCGAAGUGCAGAUCGAUCGGCUGAUCACUGGUAGGCUGCAGAGUCUG
CAGACAUAUGUCACCCAGCAGCUGAUCAGAGCAGCCGAGAUUCGAGCUUCAGCUAAC
UUAGCUGCCACAAAGAUGAGUGAGUGCGUGCUGGGCCAGUCCAAGAGGGUGGAUUU
CUGCGGAAAAGGCUAUCAUCUCAUGUCCUUCCCCCAAAGCGCACCCCACGGGGUUG
UCUUCCUGCACGUAACGUAUGUGCCCGCUCAGGAGAAGAACUUUACUACUGCCCCC
GCCAUCUGCCACGAUGGGAAAGCUCACUUCCCGAGAGAAGGAGUAUUCGUUUCCAA
UGGGACGCAUUGGUUCGUCACCCAGCGGAACUUUUACGAACCACAGAUCAUUACCAC
GGAUAAUACAUUUGUGAGCGGGAAUUGCGAUGUGGUGAUUGGAAUUGUAAAUAACA
CAGUGUACGAUCCACUGCAGUCAGAAUUAGACUCAAUUAAGGAGGAGUUGGAUAAGA
UUCAUAAAAAUGGUUCAGGUGGAUCAGGUGAUAUAGAAAAACUCCUCAAUGAACAAG
UAAAUAAGGAGAUGCAAAGUUCUAACCUGUACAUGAGCAUGUCUUCUUGGUGUUACA
CCCAUAGCCUCGAUGGAGCGGGAUUGUUCCUUUUUGACCACGCUGCGGAGGAGUAU
GAGCAUGCUAAAAAGCUGAUAAUAUUUCUCAACGAGAAUAAUGUUCCAGUGCAAUUG
ACAAGUAUAUCCGCCCCUGAGCAUAAGUUUGAAGGGCUCACACAAAUUUUCCAAAAG
GCAUACGAACACGAACAGCACAUUAGCGAGUCUAUUAACAACAUUGUUGAUCAUGCA
AUCAAGUCCAAAGAUCACGCCACGUUUAAUUUCCUCCAGUGGUAUGUAGCUGAGCAA
CAUGAGGAAGAAGUGUUGUUUAAGGAUAUUCUUGAUAAAAUUGAACUUAUUGGAAAU
GAGAACCAUGGCCUCUAUCUUGCGGACCAAUACGUCAAGGGAAUUGCCAAGUCCCG
CAAGAGUUGAUGAUAAUAGGACUAGUGGAUCCAACUGGAGACUGGGUGAAAGUGAC
UACCAGAAAGUGAGGAAGCCUAAAUAAACCUAGCGUACGUAAAAAAUGGAAAGAACC
UAGCGUACGAAAAAAAAAAAAAAAAAAAAAAAAAAAAAAAAAAAAAAAAAAAAAAAAAAA
AAAAAAAAAAAAAAAAAAAAAAAAAA
SARS-CoV2 Wuhan D614G Spike-ferritin fusion protein polypeptide sequence
(SEQ ID NO: 39)
MPLLLLLPLLWAGALASQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQSLLIVN
NATNVVIKVCEFQFCNDPFLGVYYHKNNKSWMESEFRVYSSANNCTFEYVSQPFLMDLEG
KQGNFKNLREFVFKNIDGYFKIYSKHTPINLVRDLPQGFSALEPLVDLPIGINITRFQTLLALH
RSYLTPGDSSSGWTAGAAAYYVGYLQPRTFLLKYNENGTITDAVDCALDPLSETKCTLKSF
TVEKGIYQTSNFRVQPTESIVRFPNITNLCPFGEVFNATRFASVYAWNRKRISNCVADYSVL
YNSASFSTFKCYGVSPTKLNDLCFTNVYADSFVIRGDEVRQIAPGQTGKIADYNYKLPDDFT
GCVIAWNSNNLDSKVGGNYNYLYRLFRKSNLKPFERDISTEIYQAGSTPCNGVEGENCYFP
LQSYGFQPTNGVGYQPYRVVVLSFELLHAPATVCGPKKSTNLVKNKCVNFNFNGLTGTGV
LTESNKKFLPFQQFGRDIADTTDAVRDPQTLEILDITPCSFGGVSVITPGTNTSNQVAVLYQG
VNCTEVPVAIHADQLTPTWRVYSTGSNVFQTRAGCLIGAEHVNNSYECDIPIGAGICASYQT
QTNSPGSASSVASQSIIAYTMSLGAENSVAYSNNSIAIPTNFTISVTTEILPVSMTKTSVDCTM
YICGDSTECSNLLLQYGSFCTQLNRALTGIAVEQDKNTQEVFAQVKQIYKTPPIKDFGGFNF
SQILPDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQFN
SAIGKIQDSLSSTASALGKLQDVVNQNAQALNTLVKQLSSNFGAISSVLNDILSRLDPPEAEV
QIDRLITGRLQSLQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWFVTQRNFYEPQI
ITTDNTFVSGNCDVVIGIVNNTVYDPLQSELDSIKEELDKIHKN
SARS-CoV-2 Delta Spike-ferritin fusion protein polypeptide sequence (SEQ ID NO: 40)
MPLLLLLPLLWAGALASQCVNLITRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQSLLIVN
NATNVVIKVCEFQFCNDPFLDVYYHKNNKSWMESEFRVYSSANNCTFEYVSQPFLMDLEG
KQGNFKNLREFVFKNIDGYFKIYSKHTPINLGRDLPQGFSALEPLVDLPIGINITRFQTLLALH
RSYLTPGDSSSGWTAGAAAYYVGYLQPRTFLLKYNENGTITDAVDCALDPLSETKCTLKSF
TVEKGIYQTSNFRVQPTESIVRFPNITNLCPFDEVFNATRFASVYAWNRKRISNCVADYSVL
YNFAPFFAFKCYGVSPTKLNDLCFTNVYADSFVIRGNEVSQIAPGQTGNIADYNYKLPDDFT
GCVIAWNSNKLDSKVGGNYNYRYRLFRKSNLKPFERDISTEIYQAGNKPCNGVAGVNCYFP
LQSYGFRPTYGVGHQPYRVVVLSFELLHAPATVCGPKKSTNLVKNKCVNFNFNGLTGTGVL
TESNKKFLPFQQFGRDIADTTDAVRDPQTLEILDITPCSFGGVSVITPGTNTSNQVAVLYQG
VNCTEVPVAIHADQLTPTWRVYSTGSNVFQTRAGCLIGAEYVNNSYECDIPIGAGICASYQT
QTKSHGSASSVASQSIIAYTMSLGAENSVAYSNNSIAIPTNFTISVTTEILPVSMTKTSVDCTM
YICGDSTECSNLLLQYGSFCTQLKRALTGIAVEQDKNTQEVFAQVKQIYKTPPIKYFGGFNF
SQILPDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQFN
SAIGKIQDSLSSTASALGKLQDVVNHNAQALNTLVKQLSSKFGAISSVLNDILSRLDPPEAEV
QIDRLITGRLQSLQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWFVTQRNFYEPQI
ITTDNTFVSGNCDVVIGIVNNTVYDPLQSELDSIKEELDKIHKN
SARS-CoV-2 Omicron BA.4/5 Spike-ferritin fusion protein polypeptide sequence (SEQ
ID NO: 41) (leader sequence underlined)
MPLLLLLPLLWAGALASQCVNLITRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQSLLIVN
NATNVVIKVCEFQFCNDPFLDVYYHKNNKSWMESEFRVYSSANNCTFEYVSQPFLMDLEG
KQGNFKNLREFVFKNIDGYFKIYSKHTPINLGRDLPQGFSALEPLVDLPIGINITRFQTLLALH
RSYLTPGDSSSGWTAGAAAYYVGYLQPRTFLLKYNENGTITDAVDCALDPLSETKCTLKSF
TVEKGIYQTSNFRVQPTESIVRFPNITNLCPFDEVFNATRFASVYAWNRKRISNCVADYSVL
YNFAPFFAFKCYGVSPTKLNDLCFTNVYADSFVIRGNEVSQIAPGQTGNIADYNYKLPDDFT
GCVIAWNSNKLDSKVGGNYNYRYRLFRKSNLKPFERDISTEIYQAGNKPCNGVAGVNCYFP
LQSYGFRPTYGVGHQPYRVVVLSFELLHAPATVCGPKKSTNLVKNKCVNFNFNGLTGTGVL
TESNKKFLPFQQFGRDIADTTDAVRDPQTLEILDITPCSFGGVSVITPGTNTSNQVAVLYQG
VNCTEVPVAIHADQLTPTWRVYSTGSNVFQTRAGCLIGAEYVNNSYECDIPIGAGICASYQT
QTKSHGSASSVASQSIIAYTMSLGAENSVAYSNNSIAIPTNFTISVTTEILPVSMTKTSVDCTM
YICGDSTECSNLLLQYGSFCTQLKRALTGIAVEQDKNTQEVFAQVKQIYKTPPIKYFGGFNF
SQILPDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQFN
SAIGKIQDSLSSTASALGKLQDVVNHNAQALNTLVKQLSSKFGAISSVLNDILSRLDPPEAEV
QIDRLITGRLQSLQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWFVTQRNFYEPQI
ITTDNTFVSGNCDVVIGIVNNTVYDPLQSELDSIKEELDKIHKNGSGGSGDIEKLLNEQVNKE
MQSSNLYMSMSSWCYTHSLDGAGLFLFDHAAEEYEHAKKLIIFLNENNVPVQLTSISAPEHK
FEGLTQIFQKAYEHEQHISESINNIVDHAIKSKDHATFNFLQWYVAEQHEEEVLFKDILDKIELI
GNENHGLYLADQYVKGIAKSRKS
SARS-CoV-2 XBB.1.5 Spike Protein-linker-ferritin RNA sequence (SEQ ID NO: 42)
AUGCCCCUCCUUCUCCUUCUUCCCCUCCUUUGGGCUGGAGCGCUGGCCUCUCAGUG
UGUAAAUCUCAUUACCCGCACGCAGAGCUAUACCAAUUCCUUCACACGGGGCGUGUA
CUAUCCCGACAAGGUGUUUAGAUCUAGCGUGCUGCACUCCACACAGGAUCUGUUUC
UGCCUUUCUUUUCUAACGUGACCUGGUUCCACGCCAUCCACGUGAGCGGCACCAAU
GGCACAAAGAGGUUCGACAAUCCAGCCCUGCCCUUUAACGAUGGCGUGUACUUCGC
CUCCACCGAGAAGUCUAACAUCAUCCGCGGCUGGAUCUUUGGCACCACACUGGACA
GCAAGACACAGUCCCUGCUGAUCGUGAACAAUGCCACCAACGUGGUCAUCAAGGUG
UGCGAGUUCCAGUUUUGUAAUGAUCCAUUCCUGGACGUGUAUCAGAAGAACAAUAAG
UCUUGGAUGGAGAGCGAGUUCCGGGUGUAUUCCUCUGCCAACAAUUGCACAUUUGA
GUACGUGUCCCAGCCCUUCCUGAUGGACCUGGAGGGCAAGGAGGGCAAUUUCAAGA
ACCUGCGGGAGUUCGUGUUUAAGAAUAUCGAUGGCUACUUCAAGAUCUACUCCAAG
CACACCCCCAUCAACCUGGAGCGGGACCUGCCACAGGGCUUCUCUGCCCUGGAGCC
ACUGGUGGAUCUGCCCAUCGGCAUCAACAUCACCCGGUUUCAGACACUGCUGGCCC
UGCACAGAAGCUACCUGACACCAGUGGACAGCUCCUCUGGCUGGACCGCAGGAGCA
GCAGCCUACUAUGUGGGCUAUCUGCAGCCCCGGACCUUCCUGCUGAAGUACAACGA
GAAUGGCACCAUCACAGACGCCGUGGAUUGCGCCCUGGAUCCCCUGUCUGAGACAA
AGUGUACACUGAAGAGUUUUACCGUGGAGAAGGGCAUCUAUCAGACAAGCAAUUUCA
GGGUGCAGCCUACCGAGUCCAUCGUGCGCUUUCCCAAUAUCACAAACCUGUGCCCU
UUUCACGAGGUGUUCAACGCCACCACCUUCGCCAGCGUGUACGCCUGGAAUAGGAA
GCGCAUCUCCAACUGCGUGGCCGACUAUUCUGUGAUCUACAACUUCGCCCCCUUCU
UCGCCUUUAAGUGCUAUGGCGUGAGCCCCACAAAGCUGAAUGACCUGUGCUUUACC
AACGUGUACGCCGAUUCCUUCGUGAUCAGGGGCAACGAGGUGAGCCAGAUCGCACC
AGGACAGACAGGCAACAUCGCAGACUACAAUUAUAAGCUGCCUGACGAUUUCACCGG
CUGCGUGAUCGCCUGGAACUCUAACAAGCUGGAUAGCAAACCCAGCGGCAACUACAA
UUAUCUGUACCGGCUGUUUAGAAAGUCUAAGCUGAAGCCAUUCGAGCGGGACAUCU
CCACAGAGAUCUACCAGGCCGGCAACAAGCCCUGCAAUGGCGUGGCCGGCCCAAAC
UGUUAUAGCCCAUUGCAGAGUUACGGCUUUCGGCCGACUUAUGGCGUCGGACAUCA
ACCUUACAGGGUAGUUGUUCUGUCUUUCGAACUCCUGCAUGCUCCGGCAACGGUGU
GUGGUCCCAAGAAAAGUACUAACCUUGUCAAAAAUAAGUGUGUGAACUUCAAUUUUA
ACGGUCUUACAGGUACUGGAGUAUUGACUGAGUCAAAUAAGAAGUUUCUUCCAUUCC
AACAAUUCGGCCGCGAUAUAGCUGAUACAACGGAUGCCGUCCGAGACCCUCAGACUC
UGGAGAUCCUGGACAUAACUCCAUGUUCUUUUGGAGGCGUUAGUGUGAUUACCCCA
GGCACUAAUACCUCAAACCAAGUAGCCGUCCUGUAUCAAGGAGUCAAUUGUACUGAG
GUCCCUGUGGCCAUCCAUGCGGACCAACUGACACCCACAUGGCGCGUAUAUAGCAC
AGGAUCAAACGUAUUCCAGACUCGCGCGGGUUGUCUGAUCGGGGCGGAAUACGUAA
ACAAUAGCUAUGAGUGUGAUAUACCUAUUGGAGCUGGGAUUUGCGCAAGCUAUCAAA
CACAAACGAAAUCCCACGGAUCGGCAUCAUCAGUUGCAUCUCAAUCUAUUAUCGCUU
AUACAAUGUCUCUUGGCGCUGAGAACUCCGUUGCUUACAGCAAUAAUAGCAUUGCCA
UACCGACGAACUUUACUAUUAGCGUAACGACUGAGAUACUCCCGGUGUCUAUGACAA
AAACUAGCGUUGAUUGUACAAUGUACAUUUGUGGGGAUAGUACAGAGUGCUCAAACU
UGCUUCUCCAGUACGGGUCAUUCUGUACCCAACUUAAACGCGCAUUGACUGGUAUU
GCCGUCGAGCAAGACAAAAACACACAAGAGGUUUUCGCUCAAGUAAAACAAAUUUAC
AAAACGCCCCCUAUAAAAUAUUUUGGCGGUUUCAACUUUUCUCAAAUUCUGCCCGAU
CCUUCUAAGCCUAGUAAGCGAAGCUUCAUUGAAGACCUGCUUUUCAACAAAGUAACC
UUGGCUGACGCGGGUUUCAUAAAACAGUAUGGAGACUGUCUCGGGGACAUAGCCGC
AAGGGACUUGAUUUGUGCCCAAAAAUUCAACGGACUGACAGUUCUUCCUCCACUUUU
GACUGAUGAGAUGAUAGCACAAUACACUUCUGCUUUGCUGGCCGGUACAAUAACGAG
UGGGUGGACCUUCGGGGCUGGGGCUGCCCUCCAAAUACCCUUCGCUAUGCAAAUGG
CAUAUCGAUUUAAUGGCAUCGGGGUUACACAGAACGUCCUGUAUGAAAACCAAAAGC
UCAUUGCCAACCAGUUUAACUCAGCAAUCGGAAAAAUUCAGGACAGCCUGAGUAGUA
CCGCCAGCGCCUUGGGAAAGUUGCAGGAUGUCGUCAACCAUAACGCCCAAGCAUUG
AAUACGUUGGUCAAACAGCUCUCCUCUAAGUUCGGUGCUAUAUCCUCAGUCCUCAAC
GAUAUCCUGAGCAGGCUCGAUCCCCCUGAGGCCGAAGUGCAGAUCGAUCGGCUGAU
CACUGGUAGGCUGCAGAGUCUGCAGACAUAUGUCACCCAGCAGCUGAUCAGAGCAG
CCGAGAUUCGAGCUUCAGCUAACUUAGCUGCCACAAAGAUGAGUGAGUGCGUGCUG
GGCCAGUCCAAGAGGGUGGAUUUCUGCGGAAAAGGCUAUCAUCUCAUGUCCUUCCC
CCAAAGCGCACCCCACGGGGUUGUCUUCCUGCACGUAACGUAUGUGCCCGCUCAGG
AGAAGAACUUUACUACUGCCCCCGCCAUCUGCCACGAUGGGAAAGCUCACUUCCCGA
GAGAAGGAGUAUUCGUUUCCAAUGGGACGCAUUGGUUCGUCACCCAGCGGAACUUU
UACGAACCACAGAUCAUUACCACGGAUAAUACAUUUGUGAGCGGGAAUUGCGAUGUG
GUGAUUGGAAUUGUAAAUAACACAGUGUACGAUCCACUGCAGUCAGAAUUAGACUCA
AUUAAGGAGGAGUUGGAUAAGAUUCAUAAAAAUGGUUCAGGUGGAUCAGGUGAUAUA
GAAAAACUCCUCAAUGAACAAGUAAAUAAGGAGAUGCAAAGUUCUAACCUGUACAUGA
GCAUGUCUUCUUGGUGUUACACCCAUAGCCUCGAUGGAGCGGGAUUGUUCCUUUUU
GACCACGCUGCGGAGGAGUAUGAGCAUGCUAAAAAGCUGAUAAUAUUUCUCAACGAG
AAUAAUGUUCCAGUGCAAUUGACAAGUAUAUCCGCCCCUGAGCAUAAGUUUGAAGGG
CUCACACAAAUUUUCCAAAAGGCAUACGAACACGAACAGCACAUUAGCGAGUCUAUU
AACAACAUUGUUGAUCAUGCAAUCAAGUCCAAAGAUCACGCCACGUUUAAUUUCCUC
CAGUGGUAUGUAGCUGAGCAACAUGAGGAAGAAGUGUUGUUUAAGGAUAUUCUUGA
UAAAAUUGAACUUAUUGGAAAUGAGAACCAUGGCCUCUAUCUUGCGGACCAAUACGU
CAAGGGAAUUGCCAAGUCCCGCAAGAGU
mRNA construct sequence encoding SARS-CoV-2 XBB.1.5 Spike-ferritin fusion protein
(SEQ ID NO: 43)
AGGAUUGUGCUGCAUCAAGCUUGCCGCCACCAUGCCCCUCCUUCUCCUUCUUCCCC
UCCUUUGGGCUGGAGCGCUGGCCUCUCAGUGUGUAAAUCUCAUUACCCGCACGCAG
AGCUAUACCAAUUCCUUCACACGGGGCGUGUACUAUCCCGACAAGGUGUUUAGAUC
UAGCGUGCUGCACUCCACACAGGAUCUGUUUCUGCCUUUUUUUCUAACGUGACCU
GGUUCCACGCCAUCCACGUGAGCGGCACCAAUGGCACAAAGAGGUUCGACAAUCCA
GCCCUGCCCUUUAACGAUGGCGUGUACUUCGCCUCCACCGAGAAGUCUAACAUCAU
CCGCGGCUGGAUCUUUGGCACCACACUGGACAGCAAGACACAGUCCCUGCUGAUCG
UGAACAAUGCCACCAACGUGGUCAUCAAGGUGUGCGAGUUCCAGUUUUGUAAUGAU
CCAUUCCUGGACGUGUAUCAGAAGAACAAUAAGUCUUGGAUGGAGAGCGAGUUCCG
GGUGUAUUCCUCUGCCAACAAUUGCACAUUUGAGUACGUGUCCCAGCCCUUCCUGA
UGGACCUGGAGGGCAAGGAGGGCAAUUUCAAGAACCUGCGGGAGUUCGUGUUUAAG
AAUAUCGAUGGCUACUUCAAGAUCUACUCCAAGCACACCCCCAUCAACCUGGAGCGG
GACCUGCCACAGGGCUUCUCUGCCCUGGAGCCACUGGUGGAUCUGCCCAUCGGCAU
CAACAUCACCCGGUUUCAGACACUGCUGGCCCUGCACAGAAGCUACCUGACACCAGU
GGACAGCUCCUCUGGCUGGACCGCAGGAGCAGCAGCCUACUAUGUGGGCUAUCUGC
AGCCCCGGACCUUCCUGCUGAAGUACAACGAGAAUGGCACCAUCACAGACGCCGUG
GAUUGCGCCCUGGAUCCCCUGUCUGAGACAAAGUGUACACUGAAGAGUUUUACCGU
GGAGAAGGGCAUCUAUCAGACAAGCAAUUUCAGGGUGCAGCCUACCGAGUCCAUCG
UGCGCUUUCCCAAUAUCACAAACCUGUGCCCUUUUCACGAGGUGUUCAACGCCACCA
CCUUCGCCAGCGUGUACGCCUGGAAUAGGAAGCGCAUCUCCAACUGCGUGGCCGAC
UAUUCUGUGAUCUACAACUUCGCCCCCUUCUUCGCCUUUAAGUGCUAUGGCGUGAG
CCCCACAAAGCUGAAUGACCUGUGCUUUACCAACGUGUACGCCGAUUCCUUCGUGA
UCAGGGGCAACGAGGUGAGCCAGAUCGCACCAGGACAGACAGGCAACAUCGCAGAC
UACAAUUAUAAGCUGCCUGACGAUUUCACCGGCUGCGUGAUCGCCUGGAACUCUAA
CAAGCUGGAUAGCAAACCCAGCGGCAACUACAAUUAUCUGUACCGGCUGUUUAGAAA
GUCUAAGCUGAAGCCAUUCGAGCGGGACAUCUCCACAGAGAUCUACCAGGCCGGCA
ACAAGCCCUGCAAUGGCGUGGCCGGCCCAAACUGUUAUAGCCCAUUGCAGAGUUAC
GGCUUUCGGCCGACUUAUGGCGUCGGACAUCAACCUUACAGGGUAGUUGUUCUGUC
UUUCGAACUCCUGCAUGCUCCGGCAACGGUGUGUGGUCCCAAGAAAAGUACUAACC
UUGUCAAAAAUAAGUGUGUGAACUUCAAUUUUAACGGUCUUACAGGUACUGGAGUAU
UGACUGAGUCAAAUAAGAAGUUUCUUCCAUUCCAACAAUUCGGCCGCGAUAUAGCUG
AUACAACGGAUGCCGUCCGAGACCCUCAGACUCUGGAGAUCCUGGACAUAACUCCAU
GUUCUUUUGGAGGCGUUAGUGUGAUUACCCCAGGCACUAAUACCUCAAACCAAGUA
GCCGUCCUGUAUCAAGGAGUCAAUUGUACUGAGGUCCCUGUGGCCAUCCAUGCGGA
CCAACUGACACCCACAUGGCGCGUAUAUAGCACAGGAUCAAACGUAUUCCAGACUCG
CGCGGGUUGUCUGAUCGGGGCGGAAUACGUAAACAAUAGCUAUGAGUGUGAUAUAC
CUAUUGGAGCUGGGAUUUGCGCAAGCUAUCAAACACAAACGAAAUCCCACGGAUCGG
CAUCAUCAGUUGCAUCUCAAUCUAUUAUCGCUUAUACAAUGUCUCUUGGCGCUGAGA
ACUCCGUUGCUUACAGCAAUAAUAGCAUUGCCAUACCGACGAACUUUACUAUUAGCG
UAACGACUGAGAUACUCCCGGUGUCUAUGACAAAAACUAGCGUUGAUUGUACAAUGU
ACAUUUGUGGGGAUAGUACAGAGUGCUCAAACUUGCUUCUCCAGUACGGGUCAUUC
UGUACCCAACUUAAACGCGCAUUGACUGGUAUUGCCGUCGAGCAAGACAAAAACACA
CAAGAGGUUUUCGCUCAAGUAAAACAAAUUUACAAAACGCCCCCUAUAAAAUAUUUUG
GCGGUUUCAACUUUUCUCAAAUUCUGCCCGAUCCUUCUAAGCCUAGUAAGCGAAGCU
UCAUUGAAGACCUGCUUUUCAACAAAGUAACCUUGGCUGACGCGGGUUUCAUAAAAC
AGUAUGGAGACUGUCUCGGGGACAUAGCCGCAAGGGACUUGAUUUGUGCCCAAAAA
UUCAACGGACUGACAGUUCUUCCUCCACUUUUGACUGAUGAGAUGAUAGCACAAUAC
ACUUCUGCUUUGCUGGCCGGUACAAUAACGAGUGGGUGGACCUUCGGGGCUGGGG
CUGCCCUCCAAAUACCCUUCGCUAUGCAAAUGGCAUAUCGAUUUAAUGGCAUCGGG
GUUACACAGAACGUCCUGUAUGAAAACCAAAAGCUCAUUGCCAACCAGUUUAACUCA
GCAAUCGGAAAAAUUCAGGACAGCCUGAGUAGUACCGCCAGCGCCUUGGGAAAGUU
GCAGGAUGUCGUCAACCAUAACGCCCAAGCAUUGAAUACGUUGGUCAAACAGCUCUC
CUCUAAGUUCGGUGCUAUAUCCUCAGUCCUCAACGAUAUCCUGAGCAGGCUCGAUC
CCCCUGAGGCCGAAGUGCAGAUCGAUCGGCUGAUCACUGGUAGGCUGCAGAGUCUG
CAGACAUAUGUCACCCAGCAGCUGAUCAGAGCAGCCGAGAUUCGAGCUUCAGCUAAC
UUAGCUGCCACAAAGAUGAGUGAGUGCGUGCUGGGCCAGUCCAAGAGGGUGGAUUU
CUGCGGAAAAGGCUAUCAUCUCAUGUCCUUCCCCCAAAGCGCACCCCACGGGGUUG
UCUUCCUGCACGUAACGUAUGUGCCCGCUCAGGAGAAGAACUUUACUACUGCCCCC
GCCAUCUGCCACGAUGGGAAAGCUCACUUCCCGAGAGAAGGAGUAUUCGUUUCCAA
UGGGACGCAUUGGUUCGUCACCCAGCGGAACUUUUACGAACCACAGAUCAUUACCAC
GGAUAAUACAUUUGUGAGCGGGAAUUGCGAUGUGGUGAUUGGAAUUGUAAAUAACA
CAGUGUACGAUCCACUGCAGUCAGAAUUAGACUCAAUUAAGGAGGAGUUGGAUAAGA
UUCAUAAAAAUGGUUCAGGUGGAUCAGGUGAUAUAGAAAAACUCCUCAAUGAACAAG
UAAAUAAGGAGAUGCAAAGUUCUAACCUGUACAUGAGCAUGUCUUCUUGGUGUUACA
CCCAUAGCCUCGAUGGAGCGGGAUUGUUCCUUUUUGACCACGCUGCGGAGGAGUAU
GAGCAUGCUAAAAAGCUGAUAAUAUUUCUCAACGAGAAUAAUGUUCCAGUGCAAUUG
ACAAGUAUAUCCGCCCCUGAGCAUAAGUUUGAAGGGCUCACACAAAUUUUCCAAAAG
GCAUACGAACACGAACAGCACAUUAGCGAGUCUAUUAACAACAUUGUUGAUCAUGCA
AUCAAGUCCAAAGAUCACGCCACGUUUAAUUUCCUCCAGUGGUAUGUAGCUGAGCAA
CAUGAGGAAGAAGUGUUGUUUAAGGAUAUUCUUGAUAAAAUUGAACUUAUUGGAAAU
GAGAACCAUGGCCUCUAUCUUGCGGACCAAUACGUCAAGGGAAUUGCCAAGUCCCG
CAAGAGUUGAUGAUAAUAGGACUAGUGGAUCCAACUGGAGACUGGGUGAAAGUGAC
UACCAGAAAGUGAGGAAGCCUAAAUAAACCUAGCGUACGUAAAAAAUGGAAAGAACC
UAGCGUACGAAAAAAAAAAAAAAAAAAAAAAAAAAAAAAAAAAAAAAAAAAAAAAAAAAA
AAAAAAAAAAAAAAAAAAAAAAA
SARS-CoV2 Omicron XBB.1.5 Spike-ferritin fusion protein polypeptide sequence (leader
sequence underlined) (SEQ ID NO: 44)
MPLLLLLPLLWAGALASQCVNLITRTQSYTNSFTRGVYYPDKVFRSSVLHSTQDLFLPFFSN
VTWFHAIHVSGTNGTKRFDNPALPFNDGVYFASTEKSNIIRGWIFGTTLDSKTQSLLIVNNAT
NVVIKVCEFQFCNDPFLDVYQKNNKSWMESEFRVYSSANNCTFEYVSQPFLMDLEGKEGN
FKNLREFVFKNIDGYFKIYSKHTPINLERDLPQGFSALEPLVDLPIGINITRFQTLLALHRSYLT
PVDSSSGWTAGAAAYYVGYLQPRTFLLKYNENGTITDAVDCALDPLSETKCTLKSFTVEKGI
YQTSNFRVQPTESIVRFPNITNLCPFHEVFNATTFASVYAWNRKRISNCVADYSVIYNFAPFF
AFKCYGVSPTKLNDLCFTNVYADSFVIRGNEVSQIAPGQTGNIADYNYKLPDDFTGCVIAWN
SNKLDSKPSGNYNYLYRLFRKSKLKPFERDISTEIYQAGNKPCNGVAGPNCYSPLQSYGFR
PTYGVGHQPYRVVVLSFELLHAPATVCGPKKSTNLVKNKCVNFNFNGLTGTGVLTESNKKF
LPFQQFGRDIADTTDAVRDPQTLEILDITPCSFGGVSVITPGTNTSNQVAVLYQGVNCTEVP
VAIHADQLTPTWRVYSTGSNVFQTRAGCLIGAEYVNNSYECDIPIGAGICASYQTQTKSHGS
ASSVASQSIIAYTMSLGAENSVAYSNNSIAIPTNFTISVTTEILPVSMTKTSVDCTMYICGDST
ECSNLLLQYGSFCTQLKRALTGIAVEQDKNTQEVFAQVKQIYKTPPIKYFGGFNFSQILPDPS
KPSKRSFIEDLLFNKVTLADAGFIKQYGDCLGDIAARDLICAQKFNGLTVLPPLLTDEMIAQYT
SALLAGTITSGWTFGAGAALQIPFAMQMAYRFNGIGVTQNVLYENQKLIANQFNSAIGKIQD
SLSSTASALGKLQDVVNHNAQALNTLVKQLSSKFGAISSVLNDILSRLDPPEAEVQIDRLITG
RLQSLQTYVTQQLIRAAEIRASANLAATKMSECVLGQSKRVDFCGKGYHLMSFPQSAPHGV
VFLHVTYVPAQEKNFTTAPAICHDGKAHFPREGVFVSNGTHWFVTQRNFYEPQIITTDNTFV
SGNCDVVIGIVNNTVYDPLQSELDSIKEELDKIHKNGSGGSGDIEKLLNEQVNKEMQSSNLY
MSMSSWCYTHSLDGAGLFLFDHAAEEYEHAKKLIIFLNENNVPVQLTSISAPEHKFEGLTQIF
QKAYEHEQHISESINNIVDHAIKSKDHATFNFLQWYVAEQHEEEVLFKDILDKIELIGNENHGL
YLADQYVKGIAKSRKS

1. A nucleic acid molecule comprising a 5′ untranslated region (5′-UTR), a coding sequence and a 3′ untranslated region (3′-UTR), wherein the coding sequence is operably linked to the 5′-UTR and the 3′-UTR and wherein
(i) the 5′-UTR comprises a sequence derived from a 5′-UTR of human chitinase-1 (CHIT1) and the 3′-UTR comprises a sequence derived from a 3′-UTR of human citrate synthase (CS);
(ii) the 5′-UTR comprises a sequence derived from a 5′-UTR of human protein kinase CAMP-activated catalytic subunit beta (PRKACB) and the 3′-UTR comprises a sequence derived from a 3′-UTR of human chitinase-1 (CHIT1);
(iii) the 5′-UTR comprises a sequence derived from a 5′-UTR of human protein kinase CAMP-activated catalytic subunit beta (PRKACB) and the 3′-UTR comprises a sequence derived from a 3′-UTR of human citrate synthase (CS);
(iv) the 5′-UTR comprises a sequence derived from a 5′-UTR of human chitinase-1 (CHIT1) and the 3′-UTR comprises a sequence derived from a 3′-UTR of human chitinase-1 (CHIT1), wherein the coding sequence is not derived from human chitinase-1 (CHIT1);
(v) the 5′-UTR comprises a sequence derived from a 5′-UTR of human glutamic-oxaloacetic transaminase 1 (GOT1) and the 3′-UTR comprises a sequence derived from a 3′-UTR of human citrate synthase (CS); or
(vi) the 5′-UTR comprises a sequence derived from a 5′-UTR of human glutamic-oxaloacetic transaminase 1 (GOT1) and the 3′-UTR comprises a sequence derived from a 3′-UTR of human chitinase-1 (CHIT1).

2. The nucleic acid molecule of claim 1, wherein the nucleic acid molecule is a deoxyribonucleic (DNA) molecule and (i) the 5′-UTR comprises the sequence of SEQ ID NO: 1 or a sequence at least 80%, 85%, 90% or 95% identical thereto and the 3′-UTR comprises the sequence of SEQ ID NO: 9 or a sequence at least 80%, 85%, 90% or 95% identical thereto; (ii) the 5′-UTR comprises the sequence of SEQ ID NO: 3 or a sequence at least 80%, 85%, 90% or 95% identical thereto and the 3′-UTR comprises the sequence of SEQ ID NO: 11 or a sequence at least 80%, 85%, 90% or 95% identical thereto; (iii) the 5′-UTR comprises the sequence of SEQ ID NO: 3 or a sequence at least 80%, 85%, 90% or 95% identical thereto and the 3′-UTR comprises the sequence of SEQ ID NO: 9 or a sequence at least 80%, 85%, 90% or 95% identical thereto; (iv) the 5′-UTR comprises the sequence of SEQ ID NO: 1 or a sequence at least 80%, 85%, 90% or 95% identical thereto and the 3′-UTR comprises the sequence of SEQ ID NO: 11 or a sequence at least 80%, 85%, 90% or 95% identical thereto; (v) the 5′-UTR comprises the sequence of SEQ ID NO: 5 or a sequence at least 80%, 85%, 90% or 95% identical thereto and the 3′-UTR comprises the sequence of SEQ ID NO: 9 or a sequence at least 80%, 85%, 90% or 95% identical thereto; or (vi) the 5′-UTR comprises the sequence of SEQ ID NO: 5 or a sequence at least 80%, 85%, 90% or 95% identical thereto and the 3′-UTR comprises the sequence of SEQ ID NO: 11 or a sequence at least 80%, 85%, 90% or 95% identical thereto.

3. The nucleic acid molecule of claim 1, wherein the nucleic acid molecule is a ribonucleic acid molecule and (i) the 5′-UTR comprises the sequence of SEQ ID NO: 2 or a sequence at least 80%, 85%, 90% or 95% identical thereto and the 3′-UTR comprises the sequence of SEQ ID NO: 10 or a sequence at least 80%, 85%, 90% or 95% identical thereto; (ii) the 5′-UTR comprises the sequence of SEQ ID NO: 4 or a sequence at least 80%, 85%, 90% or 95% identical thereto and the 3′-UTR comprises the sequence of SEQ ID NO: 12 or a sequence at least 80%, 85%, 90% or 95% identical thereto; (iii) the 5′-UTR comprises the sequence of SEQ ID NO: 4 or a sequence at least 80%, 85%, 90% or 95% identical thereto and the 3′-UTR comprises the sequence of SEQ ID NO: 10 or a sequence at least 80%, 85%, 90% or 95% identical thereto; (iv) the 5′-UTR comprises the sequence of SEQ ID NO: 2 or a sequence at least 80%, 85%, 90% or 95% identical thereto and the 3′-UTR comprises the sequence of SEQ ID NO: 12 or a sequence at least 80%, 85%, 90% or 95% identical thereto; (v) the 5′-UTR comprises the sequence of SEQ ID NO: 6 or a sequence at least 80%, 85%, 90% or 95% identical thereto and the 3′-UTR comprises the sequence of SEQ ID NO: 10 or a sequence at least 80%, 85%, 90% or 95% identical thereto; or (vi) the 5′-UTR comprises the sequence of SEQ ID NO: 6 or a sequence at least 80%, 85%, 90% or 95% identical thereto and the 3′-UTR comprises the sequence of SEQ ID NO: 12 or a sequence at least 80%, 85%, 90% or 95% identical thereto.

4. The nucleic acid molecule of claim 1, wherein the 5′-UTR comprises or consists of the sequence of SEQ ID NO: 2 and the 3′-UTR comprises or consists of the nucleic acid sequence of SEQ ID NO: 10.

5. The nucleic acid molecule of claim 1, wherein the coding sequence comprises a sequence encoding a therapeutic protein or peptide.

6.-10. (canceled)

11. The nucleic acid molecule of claim 1, wherein the nucleic acid molecule is mRNA.

12. The nucleic acid molecule of claim 11, wherein the coding sequence further encodes a multimerization unit (MU).

13. The nucleic acid molecule of claim 12, wherein the multimerization unit (MU) is ferritin.

14. (canceled)

15. (canceled)

16. The nucleic acid molecule of claim 5, wherein the therapeutic protein or peptide comprises a coronavirus antigen.

17. The nucleic acid molecule according to claim 16, wherein the coronavirus is selected from SARS-COV-1 and/or SARS-COV-2 and/or wherein the coronavirus antigen is a coronavirus spike (S) protein, or antigenic fragment thereof.

18. (canceled)

19. The nucleic acid molecule according to claim 17, wherein the S protein is stabilised in a pre-fusion conformation and/or wherein the S protein comprises K986P and V987P mutations.

20. (canceled)

21. (canceled)

22. A composition comprising a first nucleic acid vector comprising the molecule of claim 5, wherein the therapeutic protein or peptide comprises a Wuhan variant Spike (S) Protein, a Delta variant Spike (S) Protein, or an Omicron variant Spike (S) Protein.

23. (canceled)

24. The composition of claim 22, further comprising a second nucleic acid vector.

25. (canceled)

26. The composition of claim 24, wherein the first and/or second nucleic acid vector(s) comprises a polyadenylation sequence comprising between 60 and 100 adenine nucleotides; N1-methylpseudouridine at 80% to 100% of uridine positions; a 5′-cap structure; and/or consists of the 5′-UTR sequence set forth in SEQ ID NO: 19 and a 3′-UTR sequence set forth in SEQ ID NO: 21.

27.-30. (canceled)

31. The composition of claim 22, wherein the nucleic acid vector comprises the coding sequence set forth in SEQ ID NO: 27 or SEQ ID NO: 42.

32. The composition of claim 31, wherein the nucleic acid vector comprises a polyadenylation sequence comprising between 60 and 100 adenine nucleotides, N1-methylpseudouridine at 80% to 100% of uridine positions, a 5′-cap structure, and/or the 5′-UTR sequence set forth in SEQ ID NO: 19 and a 3′-UTR sequence set forth in SEQ ID NO: 21.

33.-35. (canceled)

36. The nucleic acid molecule of claim 11, wherein the nucleic acid molecule is formulated in lipid nanoparticles (LNPs).

37. (canceled)

38. A vaccine comprising the nucleic acid molecule of claim 1.

39. (canceled)

40. A method of preventing and/or treating an infectious disease in a subject, the method comprising administering an effective amount of the composition of claim 22 to the subject.

41. A method of vaccinating a subject against an infectious disease, comprising administering an effective amount of the composition of claims 22.

42.-59. (canceled)


SEQUENCE LISTING (44 sequences)

SEQ ID NO: 1 - DNA, 13 bp, Homo sapiens
  source 1..13: mol_type = genomic DNA
attgtgctgcatc

SEQ ID NO: 2 - RNA, 13 nt, Homo sapiens
  source 1..13: mol_type = mRNA
attgtgctgcatc

SEQ ID NO: 3 - DNA, 31 bp, Homo sapiens
  source 1..31: mol_type = genomic DNA
attctgctgtttgctccttgccaggttcaac

SEQ ID NO: 4 - RNA, 31 nt, Homo sapiens
  source 1..31: mol_type = mRNA
attctgctgtttgctccttgccaggttcaac

SEQ ID NO: 5 - DNA, 28 bp, Homo sapiens
  source 1..28: mol_type = genomic DNA
aaaatctcttgattcctagtctctcgat

SEQ ID NO: 6 - RNA, 28 nt, Homo sapiens
  source 1..28: mol_type = mRNA
aaaatctcttgattcctagtctctcgat

SEQ ID NO: 7 - DNA, 49 bp, Homo sapiens
  source 1..49: mol_type = genomic DNA
atcctcaaccaagcgccgcagacggtggccgagcgggggaccgggaagc

SEQ ID NO: 8 - RNA, 49 nt, Homo sapiens
  source 1..49: mol_type = mRNA
atcctcaaccaagcgccgcagacggtggccgagcgggggaccgggaagc

SEQ ID NO: 9 - DNA, 52 bp, Homo sapiens
  source 1..52: mol_type = genomic DNA
aactggagactgggtgaaagtgactaccagaaagtgaggaagcctaaataaa

SEQ ID NO: 10 - RNA, 52 nt, Homo sapiens
  source 1..52: mol_type = mRNA
aactggagactgggtgaaagtgactaccagaaagtgaggaagcctaaataaa

SEQ ID NO: 11 - DNA, 200 bp, Homo sapiens
  source 1..200: mol_type = genomic DNA
gtcgctaaagcccctccagtcccagctttgaggctgggcccaggatcactctacagcctgcctcctgggttttccctgggggccgcaatctggctcctgcaggcctttctgtggtcttcctttatccaggctttctgctctcagccttgccttccttttttctgggtctcctgggctgcccctttcacttgcaaaataaa

SEQ ID NO: 12 - RNA, 200 nt, Homo sapiens
  source 1..200: mol_type = mRNA
gtcgctaaagcccctccagtcccagctttgaggctgggcccaggatcactctacagcctgcctcctgggttttccctgggggccgcaatctggctcctgcaggcctttctgtggtcttcctttatccaggctttctgctctcagccttgccttccttttttctgcgactcctgggctgcccctttcacttgcaaaataaa

SEQ ID NO: 13 - DNA, 284 bp, Homo sapiens
  source 1..284: mol_type = genomic DNA
gacgcccctccctcctctggagtctacgttctccagcccacacccctccaaagccccacctttaagtcctctcttctctattcctgaccctccctacctgaggcctatctgagactataactgtcatctagccccttcgaggttgccccttccccatctccatttcacacaggtcctgaaagtctgtgtccaattatgcactggccacccaacagcaccaattgtacattccctgcatccaatctgctcagcaggccctaagatgccttgagtctttaatccca

SEQ ID NO: 14 - RNA, 284 nt, Homo sapiens
  source 1..284: mol_type = mRNA
gacgcccctccctcctctggagtctacgttctccagcccacacccctccaaagccccacctttaagtcctctcttctctattcctgaccctccctacctgaggcctatctgagactataactgtcatctagccccttcgaggttgccccttccccatctccatttcacacaggtcctgaaagtctgtgtccaattatgcactggccacccaacagcaccaattgtacattccctgcatccaatctgctcagcaggccctaagatgccttgagtctttaatccca

SEQ ID NO: 15 - DNA, 20 bp, Escherichia phage T7
  source 1..20: mol_type = genomic DNA
taatacgactcactataagg

SEQ ID NO: 16 - DNA, 187 bp, Homo sapiens
  source 1..187: mol_type = genomic DNA
gcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacct

SEQ ID NO: 17 - DNA, 61 bp, Homo sapiens
  source 1..61: mol_type = genomic DNA
gtcccgcagtcggcgtccagcggctctgcttgttcgtgtgtgtgtcgttgcaggccttatt

SEQ ID NO: 18 - DNA, 31 bp, Homo sapiens
  source 1..31: mol_type = genomic DNA
aggattgtgctgcatcaagcttgccgccacc

SEQ ID NO: 19 - RNA, 31 nt, Homo sapiens
  source 1..31: mol_type = mRNA
aggattgtgctgcatcaagcttgccgccacc

SEQ ID NO: 20 - DNA, 115 bp, Homo sapiens
  source 1..115: mol_type = genomic DNA
tgatgataataggactagtggatccaactggagactgggtgaaagtgactaccagaaagtgaggaagcctaaataaacctagcgtacgtaaaaaatggaaagaacctagcgtacg

SEQ ID NO: 21 - RNA, 115 nt, Homo sapiens
  source 1..115: mol_type = mRNA
tgatgataataggactagtggatccaactggagactgggtgaaagtgactaccagaaagtgaggaagcctaaataaacctagcgtacgtaaaaaatggaaagaacctagcgtacg

SEQ ID NO: 22 - DNA, 3993 bp, synthetic construct
  source 1..3993: mol_type = other DNA
atgcccctccttctccttcttcccctcctttgggctggagcgctggcctctcagtgtgtaaatctcaccacaagaacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagaggttcgacaatccagtgctgccctttaacgatggcgtgtacttcgcctccaccgagaagtctaacatcatccgcggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcgagtttcgggtgtattcctctgccaacaattgcacatttgagtacgtgtcccagcccttcctgatggacctggagggcaagcagggcaatttcaagaacctgcgggagttcgtgtttaagaatatcgatggctacttcaagatctactccaagcacacccccatcaacctggtgcgggacctgccacagggcttctctgccctggagccactggtggatctgcccatcggcatcaacatcacccggtttcagacactgctggccctgcacagaagctacctgacaccaggcgacagctcctctggatggaccgcaggagcagcagcctactatgtgggctatctgcagccccggaccttcctgctgaagtacaacgagaatggcaccatcacagacgccgtggattgcgccctggatcccctgtctgagacaaagtgtacactgaagagttttaccgtggagaagggcatctatcagacaagcaatttcagggtgcagcctaccgagtccatcgtgcgctttcccaatatcacaaacctgtgcccttttggcgaggtgttcaacgccaccagattcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcaccaggacagacaggcaagatcgcagactacaattataagctgcctgacgatttcaccggctgcgtgatcgcctggaactctaacaatctggatagcaaagtgggcggcaactacaattatctgtaccggctgtttagaaagtctaatctgaagccattcgagcgggacatctccacagagatctaccaggccggctctaccccctgcaatggcgtggagggctttaactgttatttccctctgcagagctacggcttccagccaaccaacggcgtgggctatcagccctacagagtggtggtgctgtcttttgagctgctgcacgcacctgcaacagtgtgcggaccaaaaaagagcaccaatctggtgaagaacaagtgcgtgaacttcaacttcaacggcctgaccggaacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcagggcgtgaattgtaccgaggtgccagtggcaatccacgcagatcagctgacccctacatggcgggtgtactctaccggcagcaacgtgttccagacaagagccggatgcctgatcggagcagagcacgtgaacaatagctatgagtgcgacatccctatcggcgccggcatctgtgcctcctaccagacccagacaaactccccagggtctgcatcatctgtggcaagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggcagcttttgtacccagctgaatcgcgccctgacaggaatcgcagtggagcaggataagaacacacaggaggtgttcgcccaggtgaagcagatctacaagaccccacccatcaaggactttggcggcttcaatttttcccagatcctgcccgatcctagcaagccatccaagaggtcttttatcgaggacctgctgttcaacaaggtgaccctggccgatgccggcttcatcaagcagtatggcgattgcctgggcgacatcgcagcccgcgacctgatctgtgcccagaagtttaatggcctgaccgtgctgcctccactgctgacagatgagatgatcgcccagtacacatctgccctgctggcaggaaccatcacaagcggatggaccttcggcgcaggagccgccctgcagatcccctttgccatgcagatggcctatcggttcaacggcatcggcgtgacccagaatgtgctgtacgagaaccagaagctgatcgccaatcagtttaactccgccatcggcaagatccaggactctctgagcagcacagcaagcgccctgggcaagctgcaggatgtggtgaatcagaacgcccaggccctgaataccctggtgaagcagctgtctagcaacttcggcgccatctcctctgtgctgaatgatatcctgagcaggctggaccctccagaggccgaagtgcagatcgatcggctgatcactggtaggctgcagagtctgcagacatatgtcacccagcagctgatcagagcagccgagattcgagcttcagctaacttagctgccacaaagatgagtgagtgcgtgctgggccagtccaagagggtggatttctgcggaaaaggctatcatctcatgtccttcccccaaagcgcaccccacggggttgtcttcctgcacgtaacgtatgtgcccgctcaggagaagaactttactactgcccccgccatctgccacgatgggaaagctcacttcccgagagaaggagtattcgtttccaatgggacgcattggttcgtcacccagcggaacttttacgaaccacagatcattaccacggataatacatttgtgagcgggaattgcgatgtggtgattggaattgtaaataacacagtgtacgatccactgcagtcagaattagactcaattaaggaggagttggataagattcataaaaatggttcaggtggatcaggtgatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagt

SEQ ID NO: 23 - RNA, 3993 nt, synthetic construct
  source 1..3993: mol_type = other RNA
atgcccctccttctccttcttcccctcctttgggctggagcgctggcctctcagtgtgtaaatctcaccacaagaacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagaggttcgacaatccagtgctgccctttaacgatggcgtgtacttcgcctccaccgagaagtctaacatcatccgcggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcgagtttcgggtgtattcctctgccaacaattgcacatttgagtacgtgtcccagcccttcctgatggacctggagggcaagcagggcaatttcaagaacctgcgggagttcgtgtttaagaatatcgatggctacttcaagatctactccaagcacacccccatcaacctggtgcgggacctgccacagggcttctctgccctggagccactggtggatctgcccatcggcatcaacatcacccggtttcagacactgctggccctgcacagaagctacctgacaccaggcgacagctcctctggatggaccgcaggagcagcagcctactatgtgggctatctgcagccccggaccttcctgctgaagtacaacgagaatggcaccatcacagacgccgtggattgcgccctggatcccctgtctgagacaaagtgtacactgaagagttttaccgtggagaagggcatctatcagacaagcaatttcagggtgcagcctaccgagtccatcgtgcgctttcccaatatcacaaacctgtgcccttttggcgaggtgttcaacgccaccagattcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcaccaggacagacaggcaagatcgcagactacaattataagctgcctgacgatttcaccggctgcgtgatcgcctggaactctaacaatctggatagcaaagtgggcggcaactacaattatctgtaccggctgtttagaaagtctaatctgaagccattcgagcgggacatctccacagagatctaccaggccggctctaccccctgcaatggcgtggagggctttaactgttatttccctctgcagagctacggcttccagccaaccaacggcgtgggctatcagccctacagagtggtggtgctgtcttttgagctgctgcacgcacctgcaacagtgtgcggaccaaaaaagagcaccaatctggtgaagaacaagtgcgtgaacttcaacttcaacggcctgaccggaacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcagggcgtgaattgtaccgaggtgccagtggcaatccacgcagatcagctgacccctacatggcgggtgtactctaccggcagcaacgtgttccagacaagagccggatgcctgatcggagcagagcacgtgaacaatagctatgagtgcgacatccctatcggcgccggcatctgtgcctcctaccagacccagacaaactccccagggtctgcatcatctgtggcaagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggcagcttttgtacccagctgaatcgcgccctgacaggaatcgcagtggagcaggataagaacacacaggaggtgttcgcccaggtgaagcagatctacaagaccccacccatcaaggactttggcggcttcaatttttcccagatcctgcccgatcctagcaagccatccaagaggtcttttatcgaggacctgctgttcaacaaggtgaccctggccgatgccggcttcatcaagcagtatggcgattgcctgggcgacatcgcagcccgcgacctgatctgtgcccagaagtttaatggcctgaccgtgctgcctccactgctgacagatgagatgatcgcccagtacacatctgccctgctggcaggaaccatcacaagcggatggaccttcggcgcaggagccgccctgcagatcccctttgccatgcagatggcctatcggttcaacggcatcggcgtgacccagaatgtgctgtacgagaaccagaagctgatcgccaatcagtttaactccgccatcggcaagatccaggactctctgagcagcacagcaagcgccctgggcaagctgcaggatgtggtgaatcagaacgcccaggccctgaataccctggtgaagcagctgtctagcaacttcggcgccatctcctctgtgctgaatgatatcctgagcaggctggaccctccagaggccgaagtgcagatcgatcggctgatcactggtaggctgcagagtctgcagacatatgtcacccagcagctgatcagagcagccgagattcgagcttcagctaacttagctgccacaaagatgagtgagtgcgtgctgggccagtccaagagggtggatttctgcggaaaaggctatcatctcatgtccttcccccaaagcgcaccccacggggttgtcttcctgcacgtaacgtatgtgcccgctcaggagaagaactttactactgcccccgccatctgccacgatgggaaagctcacttcccgagagaaggagtattcgtttccaatgggacgcattggttcgtcacccagcggaacttttacgaaccacagatcattaccacggataatacatttgtgagcgggaattgcgatgtggtgattggaattgtaaataacacagtgtacgatccactgcagtcagaattagactcaattaaggaggagttggataagattcataaaaatggttcaggtggatcaggtgatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagt

SEQ ID NO: 24 - DNA, 3987 bp, synthetic construct
  source 1..3987: mol_type = other DNA
atgcccctccttctccttcttcccctcctttgggctggagcgctggcctctcagtgtgtaaatctccgtacccgaacacaactcccccccgcctataccaacagcttcacacgcggagtttactaccctgacaaggtattcaggtccagtgtactccattcaacacaggatctgttcctcccttttttcagtaatgtgacctggtttcacgctatccacgtaagcggcacgaatggcactaagaggttcgataatcctgtactgccatttaatgatggagtttacttcgcgagcatcgagaagagcaacattatccgggggtggatctttggaacaactttggacagtaaaactcaatctctccttattgttaataacgcaaccaacgtagtcatcaaggtttgcgagttccagttttgcaatgacccttttctggacgtttattatcataagaacaacaagtcatggatggaatccggcgtgtactcttccgcaaataattgtacgtttgagtatgtcagccagcctttcctcatggacctggaaggcaaacaggggaattttaaaaatctgagggagttcgtcttcaaaaatatagacgggtactttaaaatctactccaagcacacgcccatcaatttagtgagggacctgccccaaggcttttctgctcttgaaccacttgtggaccttcccatcggcatcaatatcactcgcttccagacactcttggcacttcatagatcttatctgaccccaggggacagctcctcaggttggaccgccggcgctgcagcttactacgttggatatttgcagccacgcacgtttcttctgaaatacaatgagaatggcaccattaccgacgccgtagattgcgctctcgatcctctgagcgaaaccaaatgtacattaaagagctttacggtagaaaaggggatataccaaacctctaactttcgggtccagcctaccgaatcaatcgttcggttcccaaacattaccaacttatgtccatttggagaagtgttcaatgccacgcggttcgcaagcgtttatgcctggaacagaaagcgcatctctaattgcgtggccgattactcagtcctctacaattcagctagtttcagtacatttaaatgttacggcgtgtcccccactaagttgaatgacctttgttttactaacgtctacgctgactccttcgtgatcagaggcgatgaggttcggcagatcgctcctgggcagaccggtaagatcgccgattacaactacaaactcccagacgacttcactggctgtgtgattgcctggaacagtaacaatttagactccaaagtagggggtaactacaactatcgatatagacttttccggaaaagtaacttgaaaccttttgaaagagatatatccactgaaatctatcaggccggaagtaagccctgtaatggagtcgagggcttcaattgctacttcccattacagagctatggcttccagcctacgaacggcgtcggatatcagccttaccgcgtggtggtcttgtctttcgaactgttacatgcacctgcgacagtgtgcgggcctaagaaatccaccaacctggtgaagaacaaatgcgtgaactttaatttcaacggactgactggcacaggcgttctgactgagagcaacaagaaatttctgcccttccagcaatttggccgtgacattgccgacaccacggacgctgtacgcgatccgcaaaccctggagatcctggacatcaccccatgctcttttggcggggtcagcgttattacacctgggaccaacacaagcaaccaggttgctgtcttataccagggggttaattgcaccgaagtgcctgtcgcaatccatgctgaccaacttacccccacttggcgcgtttattctaccggctccaacgtattccaaaccagggctggctgtctcatcggagcagagcatgtgaacaactcttacgaatgcgatataccaattggcgcgggaatctgtgcctcctatcagacacaaacaaacagtcgcggaagcgcgagcagtgtggcatcacagagtatcatcgcatacaccatgagccttggcgcagagaacagcgtggcctactctaataacagtattgccatacctaccaacttcaccattagcgtgacgaccgagatcctgcccgtgtctatgaccaagacatctgtggattgcacaatgtatatctgcggagacagcactgaatgctccaatctgcttttgcagtacgggtctttctgtacccagctgaatcgagcactgacaggaatcgctgtggaacaggataagaacacccaggaggtgttcgcccaggttaagcagatatacaagacccctcctataaaagacttcggtggctttaacttttctcagattctccccgacccttctaagccttctaaacgatccttcatcgaagaccttctcttcaacaaagttactttggctgatgccgggttcatcaagcagtatggtgactgtcttggcgatatcgccgccagagatttaatatgtgcccagaagttcaacggtttaacagtccttcctcctctgctgacagacgagatgattgctcagtacactagtgctctgctcgccgggactattacttcaggctggacctttggcgctggcgccgccctgcagattccctttgctatgcaaatggcataccggttcaacggtataggtgtgactcagaatgtgctgtatgaaaatcagaaactcattgcaaatcagtttaacagtgctattgggaagatacaggactccctctcttccaccgcgtccgctttaggtaagcttcaaaacgtggtaaatcaaaatgcccaggctttaaataccttggttaagcaactgagttcaaatttcggagcaatctctagcgtgcttaacgatatcctctctaggctggatccacccgaggccgaagtgcagatcgatcggctgatcactggtaggctgcagagtctgcagacatatgtcacccagcagctgatcagagcagccgagattcgagcttcagctaacttagctgccacaaagatgagtgagtgcgtgctgggccagtccaagagggtggatttctgcggaaaaggctatcatctcatgtccttcccccaaagcgcaccccacggggttgtcttcctgcacgtaacgtatgtgcccgctcaggagaagaactttactactgcccccgccatctgccacgatgggaaagctcacttcccgagagaaggagtattcgtttccaatgggacgcattggttcgtcacccagcggaacttttacgaaccacagatcattaccacggataatacatttgtgagcgggaattgcgatgtggtgattggaattgtaaataacacagtgtacgatccactgcagtcagaattagactcaattaaggaggagttggataagattcataaaaatggttcaggtggatcaggtgatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagt

SEQ ID NO: 25 - RNA, 3987 nt, synthetic construct
  source 1..3987: mol_type = other RNA
atgcccctccttctccttcttcccctcctttgggctggagcgctggcctctcagtgtgtaaatctccgtacccgaacacaactcccccccgcctataccaacagcttcacacgcggagtttactaccctgacaaggtattcaggtccagtgtactccattcaacacaggatctgttcctcccttttttcagtaatgtgacctggtttcacgctatccacgtaagcggcacgaatggcactaagaggttcgataatcctgtactgccatttaatgatggagtttacttcgcgagcatcgagaagagcaacattatccgggggtggatctttggaacaactttggacagtaaaactcaatctctccttattgttaataacgcaaccaacgtagtcatcaaggtttgcgagttccagttttgcaatgacccttttctggacgtttattatcataagaacaacaagtcatggatggaatccggcgtgtactcttccgcaaataattgtacgtttgagtatgtcagccagcctttcctcatggacctggaaggcaaacaggggaattttaaaaatctgagggagttcgtcttcaaaaatatagacgggtactttaaaatctactccaagcacacgcccatcaatttagtgagggacctgccccaaggcttttctgctcttgaaccacttgtggaccttcccatcggcatcaatatcactcgcttccagacactcttggcacttcatagatcttatctgaccccaggggacagctcctcaggttggaccgccggcgctgcagcttactacgttggatatttgcagccacgcacgtttcttctgaaatacaatgagaatggcaccattaccgacgccgtagattgcgctctcgatcctctgagcgaaaccaaatgtacattaaagagctttacggtagaaaaggggatataccaaacctctaactttcgggtccagcctaccgaatcaatcgttcggttcccaaacattaccaacttatgtccatttggagaagtgttcaatgccacgcggttcgcaagcgtttatgcctggaacagaaagcgcatctctaattgcgtggccgattactcagtcctctacaattcagctagtttcagtacatttaaatgttacggcgtgtcccccactaagttgaatgacctttgttttactaacgtctacgctgactccttcgtgatcagaggcgatgaggttcggcagatcgctcctgggcagaccggtaagatcgccgattacaactacaaactcccagacgacttcactggctgtgtgattgcctggaacagtaacaatttagactccaaagtagggggtaactacaactatcgatatagacttttccggaaaagtaacttgaaaccttttgaaagagatatatccactgaaatctatcaggccggaagtaagccctgtaatggagtcgagggcttcaattgctacttcccattacagagctatggcttccagcctacgaacggcgtcggatatcagccttaccgcgtggtggtcttgtctttcgaactgttacatgcacctgcgacagtgtgcgggcctaagaaatccaccaacctggtgaagaacaaatgcgtgaactttaatttcaacggactgactggcacaggcgttctgactgagagcaacaagaaatttctgcccttccagcaatttggccgtgacattgccgacaccacggacgctgtacgcgatccgcaaaccctggagatcctggacatcaccccatgctcttttggcggggtcagcgttattacacctgggaccaacacaagcaaccaggttgctgtcttataccagggggttaattgcaccgaagtgcctgtcgcaatccatgctgaccaacttacccccacttggcgcgtttattctaccggctccaacgtattccaaaccagggctggctgtctcatcggagcagagcatgtgaacaactcttacgaatgcgatataccaattggcgcgggaatctgtgcctcctatcagacacaaacaaacagtcgcggaagcgcgagcagtgtggcatcacagagtatcatcgcatacaccatgagccttggcgcagagaacagcgtggcctactctaataacagtattgccatacctaccaacttcaccattagcgtgacgaccgagatcctgcccgtgtctatgaccaagacatctgtggattgcacaatgtatatctgcggagacagcactgaatgctccaatctgcttttgcagtacgggtctttctgtacccagctgaatcgagcactgacaggaatcgctgtggaacaggataagaacacccaggaggtgttcgcccaggttaagcagatatacaagacccctcctataaaagacttcggtggctttaacttttctcagattctccccgacccttctaagccttctaaacgatccttcatcgaagaccttctcttcaacaaagttactttggctgatgccgggttcatcaagcagtatggtgactgtcttggcgatatcgccgccagagatttaatatgtgcccagaagttcaacggtttaacagtccttcctcctctgctgacagacgagatgattgctcagtacactagtgctctgctcgccgggactattacttcaggctggacctttggcgctggcgccgccctgcagattccctttgctatgcaaatggcataccggttcaacggtataggtgtgactcagaatgtgctgtatgaaaatcagaaactcattgcaaatcagtttaacagtgctattgggaagatacaggactccctctcttccaccgcgtccgctttaggtaagcttcaaaacgtggtaaatcaaaatgcccaggctttaaataccttggttaagcaactgagttcaaatttcggagcaatctctagcgtgcttaacgatatcctctctaggctggatccacccgaggccgaagtgcagatcgatcggctgatcactggtaggctgcagagtctgcagacatatgtcacccagcagctgatcagagcagccgagattcgagcttcagctaacttagctgccacaaagatgagtgagtgcgtgctgggccagtccaagagggtggatttctgcggaaaaggctatcatctcatgtccttcccccaaagcgcaccccacggggttgtcttcctgcacgtaacgtatgtgcccgctcaggagaagaactttactactgcccccgccatctgccacgatgggaaagctcacttcccgagagaaggagtattcgtttccaatgggacgcattggttcgtcacccagcggaacttttacgaaccacagatcattaccacggataatacatttgtgagcgggaattgcgatgtggtgattggaattgtaaataacacagtgtacgatccactgcagtcagaattagactcaattaaggaggagttggataagattcataaaaatggttcaggtggatcaggtgatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagt

SEQ ID NO: 26 - DNA, 3993 bp, synthetic construct
  source 1..3993: mol_type = other DNA
atgcccctccttctccttcttcccctcctttgggctggagcgctggcctctcagtgtgtaaatctcattacccgcacgcagctaccacctgcctatacaaactccttcacccgcggtgtctattaccccgacaaggtttttcgatcctcagttcttcattccacccaggatcttttccttccgttcttctccaacgtcacctggtttcatgctattcatgtgtcagggaccaacggaactaaaaggttcgacaatcccgtactgcctttcaacgacggggtgtatttcgcatcaacagaaaaaagcaacattatcagaggctggatttttggcactacactcgactcaaagacccaaagtttgcttattgttaataacgcaacgaatgtcgtaattaaagtctgtgaattccagttttgtaacgacccatttctggatgtatactatcataagaacaataagtcctggatggagtcagagttcagagtctactcatcagcaaacaattgcacttttgagtacgtatctcagccatttctgatggacctggaaggcaagcaagggaacttcaaaaatctccgagagtttgttttcaaaaacatagatggttacttcaagatctattctaaacacaccccgattaatcttggccgggacctgccccagggtttctctgccctcgagccccttgtagacttgcccattgggataaatattactaggtttcagacactgcttgcgctgcacaggtcatacctcactcctggggattcatcaagcggctggactgcaggtgccgctgcatactacgtgggatacctccaaccccgaacttttttgctcaagtacaatgagaatgggacgataactgatgccgtagactgcgcactcgatcctctgtctgagacgaagtgcacactgaagagtttcacggtggaaaagggtatatatcaaacatccaatttccgagtccagcccactgaaagtattgttaggttcccgaacatcacaaatctctgtccatttgacgaggtatttaacgcgacacgctttgcttccgtgtatgcgtggaatagaaagcgcattagcaactgtgtcgcggattattcagtcttgtacaactttgcgccttttttcgcatttaaatgctacggggtcagccccacaaagctgaatgatctctgcttcacaaatgtatatgcagatagctttgtcatccgaggtaacgaagtttctcagattgctcccggccaaacgggaaacatagctgactataactataagctcccagacgattttacgggttgtgtcattgcatggaactccaataaattggattcaaaggtaggtggtaattataactacaggtataggctgttcaggaagtcaaacctgaaaccgtttgaacgagatatctcaactgagatttatcaagcgggcaataaaccctgcaacggagtcgcgggtgttaattgctacttcccattgcagagttacggctttcggccgacttatggcgtcggacatcaaccttacagggtagttgttctgtctttcgaactcctgcatgctccggcaacggtgtgtggtcccaagaaaagtactaaccttgtcaaaaataagtgtgtgaacttcaattttaacggtcttacaggtactggagtattgactgagtcaaataagaagtttcttccattccaacaattcggccgcgatatagctgatacaacggatgccgtccgagaccctcagactctggagatcctggacataactccatgttcttttggaggcgttagtgtgattaccccaggcactaatacctcaaaccaagtagccgtcctgtatcaaggagtcaattgtactgaggtccctgtggccatccatgcggaccaactgacacccacatggcgcgtatatagcacaggatcaaacgtattccagactcgcgcgggttgtctgatcggggcggaatacgtaaacaatagctatgagtgtgatatacctattggagctgggatttgcgcaagctatcaaacacaaacgaaatcccacggatcggcatcatcagttgcatctcaatctattatcgcttatacaatgtctcttggcgctgagaactccgttgcttacagcaataatagcattgccataccgacgaactttactattagcgtaacgactgagatactcccggtgtctatgacaaaaactagcgttgattgtacaatgtacatttgtggggatagtacagagtgctcaaacttgcttctccagtacgggtcattctgtacccaacttaaacgcgcattgactggtattgccgtcgagcaagacaaaaacacacaagaggttttcgctcaagtaaaacaaatttacaaaacgccccctataaaatattttggcggtttcaacttttctcaaattctgcccgatccttctaagcctagtaagcgaagcttcattgaagacctgcttttcaacaaagtaaccttggctgacgcgggtttcataaaacagtatggagactgtctcggggacatagccgcaagggacttgatttgtgcccaaaaattcaacggactgacagttcttcctccacttttgactgatgagatgatagcacaatacacttctgctttgctggccggtacaataacgagtgggtggaccttcggggctggggctgccctccaaatacccttcgctatgcaaatggcatatcgatttaatggcatcggggttacacagaacgtcctgtatgaaaaccaaaagctcattgccaaccagtttaactcagcaatcggaaaaattcaggacagcctgagtagtaccgccagcgccttgggaaagttgcaggatgtcgtcaaccataacgcccaagcattgaatacgttggtcaaacagctctcctctaagttcggtgctatatcctcagtcctcaacgatatcctgagcaggctcgatccccctgaggccgaagtgcagatcgatcggctgatcactggtaggctgcagagtctgcagacatatgtcacccagcagctgatcagagcagccgagattcgagcttcagctaacttagctgccacaaagatgagtgagtgcgtgctgggccagtccaagagggtggatttctgcggaaaaggctatcatctcatgtccttcccccaaagcgcaccccacggggttgtcttcctgcacgtaacgtatgtgcccgctcaggagaagaactttactactgcccccgccatctgccacgatgggaaagctcacttcccgagagaaggagtattcgtttccaatgggacgcattggttcgtcacccagcggaacttttacgaaccacagatcattaccacggataatacatttgtgagcgggaattgcgatgtggtgattggaattgtaaataacacagtgtacgatccactgcagtcagaattagactcaattaaggaggagttggataagattcataaaaatggttcaggtggatcaggtgatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagt

SEQ ID NO: 27 - RNA, 3993 nt, synthetic construct
  source 1..3993: mol_type = other RNA
atgcccctccttctccttcttcccctcctttgggctggagcgctggcctctcagtgtgtaaatctcattacccgcacgcagctaccacctgcctatacaaactccttcacccgcggtgtctattaccccgacaaggtttttcgatcctcagttcttcattccacccaggatcttttccttccgttcttctccaacgtcacctggtttcatgctattcatgtgtcagggaccaacggaactaaaaggttcgacaatcccgtactgcctttcaacgacggggtgtatttcgcatcaacagaaaaaagcaacattatcagaggctggatttttggcactacactcgactcaaagacccaaagtttgcttattgttaataacgcaacgaatgtcgtaattaaagtctgtgaattccagttttgtaacgacccatttctggatgtatactatcataagaacaataagtcctggatggagtcagagttcagagtctactcatcagcaaacaattgcacttttgagtacgtatctcagccatttctgatggacctggaaggcaagcaagggaacttcaaaaatctccgagagtttgttttcaaaaacatagatggttacttcaagatctattctaaacacaccccgattaatcttggccgggacctgccccagggtttctctgccctcgagccccttgtagacttgcccattgggataaatattactaggtttcagacactgcttgcgctgcacaggtcatacctcactcctggggattcatcaagcggctggactgcaggtgccgctgcatactacgtgggatacctccaaccccgaacttttttgctcaagtacaatgagaatgggacgataactgatgccgtagactgcgcactcgatcctctgtctgagacgaagtgcacactgaagagtttcacggtggaaaagggtatatatcaaacatccaatttccgagtccagcccactgaaagtattgttaggttcccgaacatcacaaatctctgtccatttgacgaggtatttaacgcgacacgctttgcttccgtgtatgcgtggaatagaaagcgcattagcaactgtgtcgcggattattcagtcttgtacaactttgcgccttttttcgcatttaaatgctacggggtcagccccacaaagctgaatgatctctgcttcacaaatgtatatgcagatagctttgtcatccgaggtaacgaagtttctcagattgctcccggccaaacgggaaacatagctgactataactataagctcccagacgattttacgggttgtgtcattgcatggaactccaataaattggattcaaaggtaggtggtaattataactacaggtataggctgttcaggaagtcaaacctgaaaccgtttgaacgagatatctcaactgagatttatcaagcgggcaataaaccctgcaacggagtcgcgggtgttaattgctacttcccattgcagagttacggctttcggccgacttatggcgtcggacatcaaccttacagggtagttgttctgtctttcgaactcctgcatgctccggcaacggtgtgtggtcccaagaaaagtactaaccttgtcaaaaataagtgtgtgaacttcaattttaacggtcttacaggtactggagtattgactgagtcaaataagaagtttcttccattccaacaattcggccgcgatatagctgatacaacggatgccgtccgagaccctcagactctggagatcctggacataactccatgttcttttggaggcgttagtgtgattaccccaggcactaatacctcaaaccaagtagccgtcctgtatcaaggagtcaattgtactgaggtccctgtggccatccatgcggaccaactgacacccacatggcgcgtatatagcacaggatcaaacgtattccagactcgcgcgggttgtctgatcggggcggaatacgtaaacaatagctatgagtgtgatatacctattggagctgggatttgcgcaagctatcaaacacaaacgaaatcccacggatcggcatcatcagttgcatctcaatctattatcgcttatacaatgtctcttggcgctgagaactccgttgcttacagcaataatagcattgccataccgacgaactttactattagcgtaacgactgagatactcccggtgtctatgacaaaaactagcgttgattgtacaatgtacatttgtggggatagtacagagtgctcaaacttgcttctccagtacgggtcattctgtacccaacttaaacgcgcattgactggtattgccgtcgagcaagacaaaaacacacaagaggttttcgctcaagtaaaacaaatttacaaaacgccccctataaaatattttggcggtttcaacttttctcaaattctgcccgatccttctaagcctagtaagcgaagcttcattgaagacctgcttttcaacaaagtaaccttggctgacgcgggtttcataaaacagtatggagactgtctcggggacatagccgcaagggacttgatttgtgcccaaaaattcaacggactgacagttcttcctccacttttgactgatgagatgatagcacaatacacttctgctttgctggccggtacaataacgagtgggtggaccttcggggctggggctgccctccaaatacccttcgctatgcaaatggcatatcgatttaatggcatcggggttacacagaacgtcctgtatgaaaaccaaaagctcattgccaaccagtttaactcagcaatcggaaaaattcaggacagcctgagtagtaccgccagcgccttgggaaagttgcaggatgtcgtcaaccataacgcccaagcattgaatacgttggtcaaacagctctcctctaagttcggtgctatatcctcagtcctcaacgatatcctgagcaggctcgatccccctgaggccgaagtgcagatcgatcggctgatcactggtaggctgcagagtctgcagacatatgtcacccagcagctgatcagagcagccgagattcgagcttcagctaacttagctgccacaaagatgagtgagtgcgtgctgggccagtccaagagggtggatttctgcggaaaaggctatcatctcatgtccttcccccaaagcgcaccccacggggttgtcttcctgcacgtaacgtatgtgcccgctcaggagaagaactttactactgcccccgccatctgccacgatgggaaagctcacttcccgagagaaggagtattcgtttccaatgggacgcattggttcgtcacccagcggaacttttacgaaccacagatcattaccacggataatacatttgtgagcgggaattgcgatgtggtgattggaattgtaaataacacagtgtacgatccactgcagtcagaattagactcaattaaggaggagttggataagattcataaaaatggttcaggtggatcaggtgatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagt

SEQ ID NO: 28 - protein, 6 aa, synthetic construct
GSGGSG

SEQ ID NO: 29 - DNA, 18 bp, synthetic construct
  source 1..18: mol_type = other DNA
ggttcaggtggatcaggt

SEQ ID NO: 30 - RNA, 18 nt, synthetic construct
  source 1..18: mol_type = other RNA
ggttcaggtggatcaggt

SEQ ID NO: 31 - DNA, 489 bp, Helicobacter pylori
  source 1..489: mol_type = genomic DNA
gatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagt

SEQ ID NO: 32 - RNA, 489 nt, Helicobacter pylori
  source 1..489: mol_type = mRNA
gatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagt

SEQ ID NO: 33 - protein, 163 aa, Helicobacter pylori
DIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 34 - protein, 16 aa, synthetic construct
MPLLLLLPLLWAGALA

SEQ ID NO: 35 - DNA, 22 bp, Homo sapiens
  source 1..22: mol_type = genomic DNA
atgggctgcagcctgccgctga

SEQ ID NO: 36 - RNA, 4216 nt, synthetic construct
  source 1..4216: mol_type = other RNA
aggattgtgctgcatcaagcttgccgccaccatgcccctccttctccttcttcccctcctttgggctggagcgctggcctctcagtgtgtaaatctcaccacaagaacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagaggttcgacaatccagtgctgccctttaacgatggcgtgtacttcgcctccaccgagaagtctaacatcatccgcggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcgagtttcgggtgtattcctctgccaacaattgcacatttgagtacgtgtcccagcccttcctgatggacctggagggcaagcagggcaatttcaagaacctgcgggagttcgtgtttaagaatatcgatggctacttcaagatctactccaagcacacccccatcaacctggtgcgggacctgccacagggcttctctgccctggagccactggtggatctgcccatcggcatcaacatcacccggtttcagacactgctggccctgcacagaagctacctgacaccaggcgacagctcctctggatggaccgcaggagcagcagcctactatgtgggctatctgcagccccggaccttcctgctgaagtacaacgagaatggcaccatcacagacgccgtggattgcgccctggatcccctgtctgagacaaagtgtacactgaagagttttaccgtggagaagggcatctatcagacaagcaatttcagggtgcagcctaccgagtccatcgtgcgctttcccaatatcacaaacctgtgcccttttggcgaggtgttcaacgccaccagattcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcaccaggacagacaggcaagatcgcagactacaattataagctgcctgacgatttcaccggctgcgtgatcgcctggaactctaacaatctggatagcaaagtgggcggcaactacaattatctgtaccggctgtttagaaagtctaatctgaagccattcgagcgggacatctccacagagatctaccaggccggctctaccccctgcaatggcgtggagggctttaactgttatttccctctgcagagctacggcttccagccaaccaacggcgtgggctatcagccctacagagtggtggtgctgtcttttgagctgctgcacgcacctgcaacagtgtgcggaccaaaaaagagcaccaatctggtgaagaacaagtgcgtgaacttcaacttcaacggcctgaccggaacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcagggcgtgaattgtaccgaggtgccagtggcaatccacgcagatcagctgacccctacatggcgggtgtactctaccggcagcaacgtgttccagacaagagccggatgcctgatcggagcagagcacgtgaacaatagctatgagtgcgacatccctatcggcgccggcatctgtgcctcctaccagacccagacaaactccccagggtctgcatcatctgtggcaagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggcagcttttgtacccagctgaatcgcgccctgacaggaatcgcagtggagcaggataagaacacacaggaggtgttcgcccaggtgaagcagatctacaagaccccacccatcaaggactttggcggcttcaatttttcccagatcctgcccgatcctagcaagccatccaagaggtcttttatcgaggacctgctgttcaacaaggtgaccctggccgatgccggcttcatcaagcagtatggcgattgcctgggcgacatcgcagcccgcgacctgatctgtgcccagaagtttaatggcctgaccgtgctgcctccactgctgacagatgagatgatcgcccagtacacatctgccctgctggcaggaaccatcacaagcggatggaccttcggcgcaggagccgccctgcagatcccctttgccatgcagatggcctatcggttcaacggcatcggcgtgacccagaatgtgctgtacgagaaccagaagctgatcgccaatcagtttaactccgccatcggcaagatccaggactctctgagcagcacagcaagcgccctgggcaagctgcaggatgtggtgaatcagaacgcccaggccctgaataccctggtgaagcagctgtctagcaacttcggcgccatctcctctgtgctgaatgatatcctgagcaggctggaccctccagaggccgaagtgcagatcgatcggctgatcactggtaggctgcagagtctgcagacatatgtcacccagcagctgatcagagcagccgagattcgagcttcagctaacttagctgccacaaagatgagtgagtgcgtgctgggccagtccaagagggtggatttctgcggaaaaggctatcatctcatgtccttcccccaaagcgcaccccacggggttgtcttcctgcacgtaacgtatgtgcccgctcaggagaagaactttactactgcccccgccatctgccacgatgggaaagctcacttcccgagagaaggagtattcgtttccaatgggacgcattggttcgtcacccagcggaacttttacgaaccacagatcattaccacggataatacatttgtgagcgggaattgcgatgtggtgattggaattgtaaataacacagtgtacgatccactgcagtcagaattagactcaattaaggaggagttggataagattcataaaaatggttcaggtggatcaggtgatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagttgatgataataggactagtggatccaactggagactgggtgaaagtgactaccagaaagtgaggaagcctaaataaacctagcgtacgtaaaaaatggaaagaacctagcgtacgaaaaaaaaaaaaaaaaaaaaaaaaaaaaaaaaaaaaaaaaaaaaaaaaaaaaaaaaaaaaaaaaaaaaaaaaaaaaa

SEQ ID NO: 37 - RNA, 4210 nt, synthetic construct
  source 1..4210: mol_type = other RNA
aggattgtgctgcatcaagcttgccgccaccatgcccctccttctccttcttcccctcctttgggctggagcgctggcctctcagtgtgtaaatctccgtacccgaacacaactcccccccgcctataccaacagcttcacacgcggagtttactaccctgacaaggtattcaggtccagtgtactccattcaacacaggatctgttcctcccttttttcagtaatgtgacctggtttcacgctatccacgtaagcggcacgaatggcactaagaggttcgataatcctgtactgccatttaatgatggagtttacttcgcgagcatcgagaaaagcaacattatccgggggtggatctttggaacaactttggacagtaaaactcaatctctccttattgttaataacgcaaccaacgtagtcatcaaggtttgcgagttccagttttgcaatgacccttttctggacgtttattatcataagaacaacaagtcatggatggaatccggcgtgtactcttccgcaaataattgtacgtttgagtatgtcagccagcctttcctcatggacctggaaggcaaacaggggaattttaaaaatctgagggagttcgtcttcaaaaatatagacgggtactttaaaatctactccaagcacacgcccatcaatttagtgagggacctgccccaaggcttttctgctcttgaaccacttgtggaccttcccatcggcatcaatatcactcgcttccagacactcttggcacttcatagatcttatctgaccccaggggacagctcctcaggttggaccgccggcgctgcagcttactacgttggatatttgcagccacgcacgtttcttctgaaatacaatgagaatggcaccattaccgacgccgtagattgcgctctcgatcctctgagcgaaaccaaatgtacattaaagagctttacggtagaaaaggggatataccaaacctctaactttcgggtccagcctaccgaatcaatcgttcggttcccaaacattaccaacttatgtccatttggagaagtgttcaatgccacgcggttcgcaagcgtttatgcctggaacagaaagcgcatctctaattgcgtggccgattactcagtcctctacaattcagctagtttcagtacatttaaatgttacggcgtgtcccccactaagttgaatgacctttgttttactaacgtctacgctgactccttcgtgatcagaggcgatgaggttcggcagatcgctcctgggcagaccggtaagatcgccgattacaactacaaactcccagacgacttcactggctgtgtgattgcctggaacagtaacaatttagactccaaagtagggggtaactacaactatcgatatagacttttccggaaaagtaacttgaaaccttttgaaagagatatatccactgaaatctatcaggccggaagtaagccctgtaatggagtcgagggcttcaattgctacttcccattacagagctatggcttccagcctacgaacggcgtcggatatcagccttaccgcgtggtggtcttgtctttcgaactgttacatgcacctgcgacagtgtgcgggcctaagaaatccaccaacctggtgaagaacaaatgcgtgaactttaatttcaacggactgactggcacaggcgttctgactgagagcaacaagaaatttctgcccttccagcaatttggccgtgacattgccgacaccacggacgctgtacgcgatccgcaaaccctggagatcctggacatcaccccatgctcttttggcggggtcagcgttattacacctgggaccaacacaagcaaccaggttgctgtcttataccagggggttaattgcaccgaagtgcctgtcgcaatccatgctgaccaacttacccccacttggcgcgtttattctaccggctccaacgtattccaaaccagggctggctgtctcatcggagcagagcatgtgaacaactcttacgaatgcgatataccaattggcgcgggaatctgtgcctcctatcagacacaaacaaacagtcgcggaagcgcgagcagtgtggcatcacagagtatcatcgcatacaccatgagccttggcgcagagaacagcgtggcctactctaataacagtattgccatacctaccaacttcaccattagcgtgacgaccgagatcctgcccgtgtctatgaccaagacatctgtggattgcacaatgtatatctgcggagacagcactgaatgctccaatctgcttttgcagtacgggtctttctgtacccagctgaatcgagcactgacaggaatcgctgtggaacaggataagaacacccaggaggtgttcgcccaggttaagcagatatacaagacccctcctataaaagacttcggtggctttaacttttctcagattctccccgacccttctaagccttctaaacgatccttcatcgaagaccttctcttcaacaaagttactttggctgatgccgggttcatcaagcagtatggtgactgtcttggcgatatcgccgccagagatttaatatgtgcccagaagttcaacggtttaacagtccttcctcctctgctgacagacgagatgattgctcagtacactagtgctctgctcgccgggactattacttcaggctggacctttggcgctggcgccgccctgcagattccctttgctatgcaaatggcataccggttcaacggtataggtgtgactcagaatgtgctgtatgaaaatcagaaactcattgcaaatcagtttaacagtgctattgggaagatacaggactccctctcttccaccgcgtccgctttaggtaagcttcaaaacgtggtaaatcaaaatgcccaggctttaaataccttggttaagcaactgagttcaaatttcggagcaatctctagcgtgcttaacgatatcctctctaggctggatccacccgaggccgaagtgcagatcgatcggctgatcactggtaggctgcagagtctgcagacatatgtcacccagcagctgatcagagcagccgagattcgagcttcagctaacttagctgccacaaagatgagtgagtgcgtgctgggccagtccaagagggtggatttctgcggaaaaggctatcatctcatgtccttcccccaaagcgcaccccacggggttgtcttcctgcacgtaacgtatgtgcccgctcaggagaagaactttactactgcccccgccatctgccacgatgggaaagctcacttcccgagagaaggagtattcgtttccaatgggacgcattggttcgtcacccagcggaacttttacgaaccacagatcattaccacggataatacatttgtgagcgggaattgcgatgtggtgattggaattgtaaataacacagtgtacgatccactgcagtcagaattagactcaattaaggaggagttggataagattcataaaaatggttcaggtggatcaggtgatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagttgatgataataggactagtggatccaactggagactgggtgaaagtgactaccagaaagtgaggaagcctaaataaacctagcgtacgtaaaaaatggaaagaacctagcgtacgaaaaaaaaaaaaaaaaaaaaaaaaaaaaaaaaaaaaaaaaaaaaaaaaaaaaaaaaaaaaaaaaaaaaaaaaaaaaa

SEQ ID NO: 38 - RNA, 4216 nt, synthetic construct
  source 1..4216: mol_type = other RNA
aggattgtgctgcatcaagcttgccgccaccatgcccctccttctccttcttcccctcctttgggctggagcgctggcctctcagtgtgtaaatctcattacccgcacgcagctaccacctgcctatacaaactccttcacccgcggtgtctattaccccgacaaggtttttcgatcctcagttcttcattccacccaggatcttttccttccgttcttctccaacgtcacctggtttcatgctattcatgtgtcagggaccaacggaactaaaaggttcgacaatcccgtactgcctttcaacgacggggtgtatttcgcatcaacagaaaaaagcaacattatcagaggctggatttttggcactacactcgactcaaagacccaaagtttgcttattgttaataacgcaacgaatgtcgtaattaaagtctgtgaattccagttttgtaacgacccatttctggatgtatactatcataagaacaataagtcctggatggagtcagagttcagagtctactcatcagcaaacaattgcacttttgagtacgtatctcagccatttctgatggacctggaaggcaagcaagggaacttcaaaaatctccgagagtttgttttcaaaaacatagatggttacttcaagatctattctaaacacaccccgattaatcttggccgggacctgccccagggtttctctgccctcgagccccttgtagacttgcccattgggataaatattactaggtttcagacactgcttgcgctgcacaggtcatacctcactcctggggattcatcaagcggctggactgcaggtgccgctgcatactacgtgggatacctccaaccccgaacttttttgctcaagtacaatgagaatgggacgataactgatgccgtagactgcgcactcgatcctctgtctgagacgaagtgcacactgaagagtttcacggtggaaaagggtatatatcaaacatccaatttccgagtccagcccactgaaagtattgttaggttcccgaacatcacaaatctctgtccatttgacgaggtatttaacgcgacacgctttgcttccgtgtatgcgtggaatagaaagcgcattagcaactgtgtcgcggattattcagtcttgtacaactttgcgccttttttcgcatttaaatgctacggggtcagccccacaaagctgaatgatctctgcttcacaaatgtatatgcagatagctttgtcatccgaggtaacgaagtttctcagattgctcccggccaaacgggaaacatagctgactataactataagctcccagacgattttacgggttgtgtcattgcatggaactccaataaattggattcaaaggtaggtggtaattataactacaggtataggctgttcaggaagtcaaacctgaaaccgtttgaacgagatatctcaactgagatttatcaagcgggcaataaaccctgcaacggagtcgcgggtgttaattgctacttcccattgcagagttacggctttcggccgacttatggcgtcggacatcaaccttacagggtagttgttctgtctttcgaactcctgcatgctccggcaacggtgtgtggtcccaagaaaagtactaaccttgtcaaaaataagtgtgtgaacttcaattttaacggtcttacaggtactggagtattgactgagtcaaataagaagtttcttccattccaacaattcggccgcgatatagctgatacaacggatgccgtccgagaccctcagactctggagatcctggacataactccatgttcttttggaggcgttagtgtgattaccccaggcactaatacctcaaaccaagtagccgtcctgtatcaaggagtcaattgtactgaggtccctgtggccatccatgcggaccaactgacacccacatggcgcgtatatagcacaggatcaaacgtattccagactcgcgcgggttgtctgatcggggcggaatacgtaaacaatagctatgagtgtgatatacctattggagctgggatttgcgcaagctatcaaacacaaacgaaatcccacggatcggcatcatcagttgcatctcaatctattatcgcttatacaatgtctcttggcgctgagaactccgttgcttacagcaataatagcattgccataccgacgaactttactattagcgtaacgactgagatactcccggtgtctatgacaaaaactagcgttgattgtacaatgtacatttgtggggatagtacagagtgctcaaacttgcttctccagtacgggtcattctgtacccaacttaaacgcgcattgactggtattgccgtcgagcaagacaaaaacacacaagaggttttcgctcaagtaaaacaaatttacaaaacgccccctataaaatattttggcggtttcaacttttctcaaattctgcccgatccttctaagcctagtaagcgaagcttcattgaagacctgcttttcaacaaagtaaccttggctgacgcgggtttcataaaacagtatggagactgtctcggggacatagccgcaagggacttgatttgtgcccaaaaattcaacggactgacagttcttcctccacttttgactgatgagatgatagcacaatacacttctgctttgctggccggtacaataacgagtgggtggaccttcggggctggggctgccctccaaatacccttcgctatgcaaatggcatatcgatttaatggcatcggggttacacagaacgtcctgtatgaaaaccaaaagctcattgccaaccagtttaactcagcaatcggaaaaattcaggacagcctgagtagtaccgccagcgccttgggaaagttgcaggatgtcgtcaaccataacgcccaagcattgaatacgttggtcaaacagctctcctctaagttcggtgctatatcctcagtcctcaacgatatcctgagcaggctcgatccccctgaggccgaagtgcagatcgatcggctgatcactggtaggctgcagagtctgcagacatatgtcacccagcagctgatcagagcagccgagattcgagcttcagctaacttagctgccacaaagatgagtgagtgcgtgctgggccagtccaagagggtggatttctgcggaaaaggctatcatctcatgtccttcccccaaagcgcaccccacggggttgtcttcctgcacgtaacgtatgtgcccgctcaggagaagaactttactactgcccccgccatctgccacgatgggaaagctcacttcccgagagaaggagtattcgtttccaatgggacgcattggttcgtcacccagcggaacttttacgaaccacagatcattaccacggataatacatttgtgagcgggaattgcgatgtggtgattggaattgtaaataacacagtgtacgatccactgcagtcagaattagactcaattaaggaggagttggataagattcataaaaatggttcaggtggatcaggtgatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagttgatgataataggactagtggatccaactggagactgggtgaaagtgactaccagaaagtgaggaagcctaaataaacctagcgtacgtaaaaaatggaaagaacctagcgtacgaaaaaaaaaaaaaaaaaaaaaaaaaaaaaaaaaaaaaaaaaaaaaaaaaaaaaaaaaaaaaaaaaaaaaaaaaaaaa

SEQ ID NO: 39 - protein, 1331 aa, synthetic construct
MPLLLLLPLLWAGALASQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQSELDSIKEELDKIHKNGSGGSGDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 40 - protein, 1329 aa, synthetic construct
MPLLLLLPLLWAGALASQCVNLRTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQSELDSIKEELDKIHKNGSGGSGDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 41 - protein, 1331 aa, synthetic construct
MPLLLLLPLLWAGALASQCVNLITRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQSELDSIKEELDKIHKNGSGGSGDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 42 - RNA, 3981 nt, synthetic construct
  source 1..3981: mol_type = other RNA
atgcccctccttctccttcttcccctcctttgggctggagcgctggcctctcagtgtgtaaatctcattacccgcacgcagagctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagaggttcgacaatccagccctgccctttaacgatggcgtgtacttcgcctccaccgagaagtctaacatcatccgcggctggatctttggcaccacactggacagcaagacacagtccctgctgatcgtgaacaatgccaccaacgtggtcatcaaggtgtgcgagttccagttttgtaatgatccattcctggacgtgtatcagaagaacaataagtcttggatggagagcgagttccgggtgtattcctctgccaacaattgcacatttgagtacgtgtcccagcccttcctgatggacctggagggcaaggagggcaatttcaagaacctgcgggagttcgtgtttaagaatatcgatggctacttcaagatctactccaagcacacccccatcaacctggagcgggacctgccacagggcttctctgccctggagccactggtggatctgcccatcggcatcaacatcacccggtttcagacactgctggccctgcacagaagctacctgacaccagtggacagctcctctggctggaccgcaggagcagcagcctactatgtgggctatctgcagccccggaccttcctgctgaagtacaacgagaatggcaccatcacagacgccgtggattgcgccctggatcccctgtctgagacaaagtgtacactgaagagttttaccgtggagaagggcatctatcagacaagcaatttcagggtgcagcctaccgagtccatcgtgcgctttcccaatatcacaaacctgtgcccttttcacgaggtgttcaacgccaccaccttcgccagcgtgtacgcctggaataggaagcgcatctccaactgcgtggccgactattctgtgatctacaacttcgcccccttcttcgcctttaagtgctatggcgtgagccccacaaagctgaatgacctgtgctttaccaacgtgtacgccgattccttcgtgatcaggggcaacgaggtgagccagatcgcaccaggacagacaggcaacatcgcagactacaattataagctgcctgacgatttcaccggctgcgtgatcgcctggaactctaacaagctggatagcaaacccagcggcaactacaattatctgtaccggctgtttagaaagtctaagctgaagccattcgagcgggacatctccacagagatctaccaggccggcaacaagccctgcaatggcgtggccggcccaaactgttatagcccattgcagagttacggctttcggccgacttatggcgtcggacatcaaccttacagggtagttgttctgtctttcgaactcctgcatgctccggcaacggtgtgtggtcccaagaaaagtactaaccttgtcaaaaataagtgtgtgaacttcaattttaacggtcttacaggtactggagtattgactgagtcaaataagaagtttcttccattccaacaattcggccgcgatatagctgatacaacggatgccgtccgagaccctcagactctggagatcctggacataactccatgttcttttggaggcgttagtgtgattaccccaggcactaatacctcaaaccaagtagccgtcctgtatcaaggagtcaattgtactgaggtccctgtggccatccatgcggaccaactgacacccacatggcgcgtatatagcacaggatcaaacgtattccagactcgcgcgggttgtctgatcggggcggaatacgtaaacaatagctatgagtgtgatatacctattggagctgggatttgcgcaagctatcaaacacaaacgaaatcccacggatcggcatcatcagttgcatctcaatctattatcgcttatacaatgtctcttggcgctgagaactccgttgcttacagcaataatagcattgccataccgacgaactttactattagcgtaacgactgagatactcccggtgtctatgacaaaaactagcgttgattgtacaatgtacatttgtggggatagtacagagtgctcaaacttgcttctccagtacgggtcattctgtacccaacttaaacgcgcattgactggtattgccgtcgagcaagacaaaaacacacaagaggttttcgctcaagtaaaacaaatttacaaaacgccccctataaaatattttggcggtttcaacttttctcaaattctgcccgatccttctaagcctagtaagcgaagcttcattgaagacctgcttttcaacaaagtaaccttggctgacgcgggtttcataaaacagtatggagactgtctcggggacatagccgcaagggacttgatttgtgcccaaaaattcaacggactgacagttcttcctccacttttgactgatgagatgatagcacaatacacttctgctttgctggccggtacaataacgagtgggtggaccttcggggctggggctgccctccaaatacccttcgctatgcaaatggcatatcgatttaatggcatcggggttacacagaacgtcctgtatgaaaaccaaaagctcattgccaaccagtttaactcagcaatcggaaaaattcaggacagcctgagtagtaccgccagcgccttgggaaagttgcaggatgtcgtcaaccataacgcccaagcattgaatacgttggtcaaacagctctcctctaagttcggtgctatatcctcagtcctcaacgatatcctgagcaggctcgatccccctgaggccgaagtgcagatcgatcggctgatcactggtaggctgcagagtctgcagacatatgtcacccagcagctgatcagagcagccgagattcgagcttcagctaacttagctgccacaaagatgagtgagtgcgtgctgggccagtccaagagggtggatttctgcggaaaaggctatcatctcatgtccttcccccaaagcgcaccccacggggttgtcttcctgcacgtaacgtatgtgcccgctcaggagaagaactttactactgcccccgccatctgccacgatgggaaagctcacttcccgagagaaggagtattcgtttccaatgggacgcattggttcgtcacccagcggaacttttacgaaccacagatcattaccacggataatacatttgtgagcgggaattgcgatgtggtgattggaattgtaaataacacagtgtacgatccactgcagtcagaattagactcaattaaggaggagttggataagattcataaaaatggttcaggtggatcaggtgatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagt

SEQ ID NO: 43 - RNA, 4201 nt, synthetic construct
  source 1..4201: mol_type = other RNA
aggattgtgctgcatcaagcttgccgccaccatgcccctccttctccttcttcccctcctttgggctggagcgctggcctctcagtgtgtaaatctcattacccgcacgcagagctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagaggttcgacaatccagccctgccctttaacgatggcgtgtacttcgcctccaccgagaagtctaacatcatccgcggctggatctttggcaccacactggacagcaagacacagtccctgctgatcgtgaacaatgccaccaacgtggtcatcaaggtgtgcgagttccagttttgtaatgatccattcctggacgtgtatcagaagaacaataagtcttggatggagagcgagttccgggtgtattcctctgccaacaattgcacatttgagtacgtgtcccagcccttcctgatggacctggagggcaaggagggcaatttcaagaacctgcgggagttcgtgtttaagaatatcgatggctacttcaagatctactccaagcacacccccatcaacctggagcgggacctgccacagggcttctctgccctggagccactggtggatctgcccatcggcatcaacatcacccggtttcagacactgctggccctgcacagaagctacctgacaccagtggacagctcctctggctggaccgcaggagcagcagcctactatgtgggctatctgcagccccggaccttcctgctgaagtacaacgagaatggcaccatcacagacgccgtggattgcgccctggatcccctgtctgagacaaagtgtacactgaagagttttaccgtggagaagggcatctatcagacaagcaatttcagggtgcagcctaccgagtccatcgtgcgctttcccaatatcacaaacctgtgcccttttcacgaggtgttcaacgccaccaccttcgccagcgtgtacgcctggaataggaagcgcatctccaactgcgtggccgactattctgtgatctacaacttcgcccccttcttcgcctttaagtgctatggcgtgagccccacaaagctgaatgacctgtgctttaccaacgtgtacgccgattccttcgtgatcaggggcaacgaggtgagccagatcgcaccaggacagacaggcaacatcgcagactacaattataagctgcctgacgatttcaccggctgcgtgatcgcctggaactctaacaagctggatagcaaacccagcggcaactacaattatctgtaccggctgtttagaaagtctaagctgaagccattcgagcgggacatctccacagagatctaccaggccggcaacaagccctgcaatggcgtggccggcccaaactgttatagcccattgcagagttacggctttcggccgacttatggcgtcggacatcaaccttacagggtagttgttctgtctttcgaactcctgcatgctccggcaacggtgtgtggtcccaagaaaagtactaaccttgtcaaaaataagtgtgtgaacttcaattttaacggtcttacaggtactggagtattgactgagtcaaataagaagtttcttccattccaacaattcggccgcgatatagctgatacaacggatgccgtccgagaccctcagactctggagatcctggacataactccatgttcttttggaggcgttagtgtgattaccccaggcactaatacctcaaaccaagtagccgtcctgtatcaaggagtcaattgtactgaggtccctgtggccatccatgcggaccaactgacacccacatggcgcgtatatagcacaggatcaaacgtattccagactcgcgcgggttgtctgatcggggcggaatacgtaaacaatagctatgagtgtgatatacctattggagctgggatttgcgcaagctatcaaacacaaacgaaatcccacggatcggcatcatcagttgcatctcaatctattatcgcttatacaatgtctcttggcgctgagaactccgttgcttacagcaataatagcattgccataccgacgaactttactattagcgtaacgactgagatactcccggtgtctatgacaaaaactagcgttgattgtacaatgtacatttgtggggatagtacagagtgctcaaacttgcttctccagtacgggtcattctgtacccaacttaaacgcgcattgactggtattgccgtcgagcaagacaaaaacacacaagaggttttcgctcaagtaaaacaaatttacaaaacgccccctataaaatattttggcggtttcaacttttctcaaattctgcccgatccttctaagcctagtaagcgaagcttcattgaagacctgcttttcaacaaagtaaccttggctgacgcgggtttcataaaacagtatggagactgtctcggggacatagccgcaagggacttgatttgtgcccaaaaattcaacggactgacagttcttcctccacttttgactgatgagatgatagcacaatacacttctgctttgctggccggtacaataacgagtgggtggaccttcggggctggggctgccctccaaatacccttcgctatgcaaatggcatatcgatttaatggcatcggggttacacagaacgtcctgtatgaaaaccaaaagctcattgccaaccagtttaactcagcaatcggaaaaattcaggacagcctgagtagtaccgccagcgccttgggaaagttgcaggatgtcgtcaaccataacgcccaagcattgaatacgttggtcaaacagctctcctctaagttcggtgctatatcctcagtcctcaacgatatcctgagcaggctcgatccccctgaggccgaagtgcagatcgatcggctgatcactggtaggctgcagagtctgcagacatatgtcacccagcagctgatcagagcagccgagattcgagcttcagctaacttagctgccacaaagatgagtgagtgcgtgctgggccagtccaagagggtggatttctgcggaaaaggctatcatctcatgtccttcccccaaagcgcaccccacggggttgtcttcctgcacgtaacgtatgtgcccgctcaggagaagaactttactactgcccccgccatctgccacgatgggaaagctcacttcccgagagaaggagtattcgtttccaatgggacgcattggttcgtcacccagcggaacttttacgaaccacagatcattaccacggataatacatttgtgagcgggaattgcgatgtggtgattggaattgtaaataacacagtgtacgatccactgcagtcagaattagactcaattaaggaggagttggataagattcataaaaatggttcaggtggatcaggtgatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagttgatgataataggactagtggatccaactggagactgggtgaaagtgactaccagaaagtgaggaagcctaaataaacctagcgtacgtaaaaaatggaaagaacctagcgtacgaaaaaaaaaaaaaaaaaaaaaaaaaaaaaaaaaaaaaaaaaaaaaaaaaaaaaaaaaaaaaaaaaaaaaaaaaa

SEQ ID NO: 44 - protein, 1327 aa, synthetic construct
MPLLLLLPLLWAGALASQCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQSELDSIKEELDKIHKNGSGGSGDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS